(19) United States
(12) Patent Application Publication
(10) Pub. No.: US 2009/0130165 A1
(43) Pub. Date: May 21, 2009

(54) MIF Inhibitors

(75) Inventors: Eric Francis Morand, Elwood (AU); Colin Edward Skene, Glen Waverly (AU); Peter Mark Tapley, Mount Waverly (AU); Xinhua Li, Waban, MA (US); Thomas H. Jozefiak, Belmont, MA (US)

Correspondence Address: STERNE, KESSLER, GOLDSTEIN & FOX P.L.L.C., 1100 NEW YORK AVENUE, N.W., WASHINGTON, DC, 20005, US

(73) Assignee: Cortical Pty Ltd

(21) Appl. No.: 12/158,563

(22) PCT Filed: Dec. 21, 2006
(86) PCT No.: PCT/AU2006/001965

§ 371 Date: Dec. 23, 2008

Related U.S. Application Data
(60) Provisional application No. 60/752,354, filed on Dec. 21, 2005.

(51) Int. Cl. A61F 2/00; A61K 31/403; A61K 31/4184; A61K 31/423; A61K 31/428; A61K 31/56

(52) U.S. Cl. 424/423; 514/418; 514/395; 514/375; 514/367; 514/171

(57) ABSTRACT

The present invention relates to the use of specific benzimidazolone analogues and derivatives to inhibit the cytokine or biological activity of macrophage migration inhibitory factor (MIF), and diseases or conditions wherein MIF cytokine or biological activity is implicated. Novel benzimidazole analogues and derivatives are also provided.

### FIELD OF THE INVENTION

The present invention relates generally to the treatment of diseases or conditions resulting from cellular activation, such as inflammatory or cancerous diseases or conditions. In particular, the invention relates to the use of specific benzimidazolone analogues and derivatives to inhibit the cytokine or biological activity of macrophage migration inhibitory factor (MIF), and diseases or conditions wherein MIF cytokine or biological activity is implicated.

### BACKGROUND OF THE INVENTION

MIF is the first identified T-cell-derived soluble lymphokine. MIF was first described as a soluble factor with the ability to modify the migration of macrophages⁽¹⁾. The molecule responsible for the biological actions ascribed to MIF was identified and cloned in 1989⁽²⁾. Initially found to activate macrophages at inflammatory sites, it has been shown to possess pluripotential actions in the immune system. MIF has been shown to be expressed in human diseases which include inflammation, injury, ischaemia or malignancy. MIF also has a unique relationship with glucocorticoids by overriding their anti-inflammatory effects.

Recent studies have indicated that monoclonal antibody antagonism of MIF may be useful in the treatment of sepsis, certain types of cancers and delayed type hypersensitivity. Antibody antagonism of MIF has also been shown to have activity in adjuvant- or collagen-induced arthritis animal models and models of other inflammatory and immune diseases including colitis, multiple sclerosis, atherosclerosis, glomerulonephritis, and uveitis.

Although antibody antagonism of MIF is one potential way to provide therapeutic treatments, such biological molecules can be expensive to prepare on a commercial basis and further, can be limited in the way they are administered (generally by injection) and do not readily lend themselves to formulations for administration by other means eg oral administration.

Small molecule inhibitors may overcome one or more such difficulties connected with the use of biological therapeutic treatments. There exists a need, therefore, for small molecule inhibitors of the cytokine or biological activity of MIF. Small molecule inhibitors of the cytokine or biological activity of MIF would have therapeutic effects in a broad range of diseases, whether given alone or in combination with other therapies.

Further, glucocorticoids have been used to treat human diseases for over fifty years and are effective in a range of diseases which include inflammation, injury, ischaemia or malignancy. Although debate continues in relation to their impact on disease progression, their influence on symptoms and signs of inflammation, especially in the short term, can be dramatic.

Despite their benefits and efficacy, the use of glucocorticoids is limited by universal, predictable, dose-dependent toxicity. Mimicking Cushing's disease, a disease wherein the adrenal glands produce excess endogenous glucocorticoids, glucocorticoid treatment is associated with side effects including immunosuppression (resulting in increased susceptibility to infections), weight gain, change in body habitus, hypertension, oedema, diabetes mellitus, cataracts, osteoporosis, poor wound healing, thinning of the skin, vascular fragility, hirsutism and other features of masculinization (in females). In children, growth retardation is also noted. These side effects are known as Cushingoid side effects.

Since the side effects of glucocorticoids are dose dependent, attempts to reduce the dosage requirement have been investigated, including combination therapies in which glucocorticoids are administered with other therapeutic agents. These combination therapies are sometimes referred to as “steroid-sparing” therapies. However, currently available combination therapies are non-specific as the other therapeutic agents do not address biological events which inhibit the effectiveness of glucocorticoids. Such combination therapies are also typically associated with serious side effects.

Furthermore, glucocorticoids are incompletely effective in a number of disease settings, leading to the concept of “steroid-resistant” diseases. Agents which amplify or enhance the effects of glucocorticoids would not only allow the reduction of dose of these agents but may also potentially fender “steroid-resistant” diseases steroid-sensitive.

There is a need for effective therapies which enable a reduction in the dosage level of glucocorticoids. There is also a need for effective treatment of “steroid-resistant” diseases. Preferably, such therapies or treatments would address factors which directly limit the effectiveness of glucocorticoids.

Therapeutic antagonism of MIF may provide “steroid-sparing” effects or be therapeutic in “steroid-resistant” diseases. Unlike other pro-inflammatory molecules, such as cytokines, the expression and/or release of MIF can be induced by glucocorticoids^{(3),(4)}. Moreover, MIF is able to directly antagonize the effects of glucocorticoids. This has been shown to be the case for macrophage TNF, IL-1β, IL-6 and IL-8 secretion^{(5),(6)}, and for T cell proliferation and IL-2 release⁽⁷⁾. In vivo, MIF exerts a powerful glucocorticoid-antagonist effect in models including endotoxic shock and experimental arthritis^{(5),(8)}. In the context of an inflammatory or other disease treated with glucocorticoids, then, MIF is expressed but exerts an effect which prevents the glucocorticoid inhibition of inflammation. It can therefore be proposed that therapeutic antagonism of MIF would remove MIF's role in inhibiting the anti-inflammatory effect of glucocorticoids, thereby allowing glucocorticoids to prevail. This would be the first example of true “steroid-sparing” therapy. In support of this hypothesis is the observation that anti-MIF antibody therapy reverses the effect of adrenalectomy in rat adjuvant arthritis⁽⁹⁾. In further support of this, it has recently been demonstrated that reduced MIF activity is indeed directly associated with improvements in responsiveness to glucocorticoids^{(20,21)}. By neutralizing the natural glucocorticoid ‘counter-regulator’ effect of MIF, it is envisioned that with MIF antagonism, steroid dosages could be reduced or even eliminated in inflammatory disease, particularly in those diseases that are associated with the glucocorticoid resistance^{(10),(11)}. There is a need, therefore, for therapeutic antagonists of the cytokine or biological activity of MIF.

MIF has recently been shown to be important in the control of leukocyte-endothelial interactions. Leukocytes interact with vascular endothelial cells in order to gain egress from the vasculature into tissues. The role of MIF in this process has been demonstrated to affect in particular leukocyte-endothelial adhesion and emigration^{(22,23)}. This process is an essential part of nearly all inflammatory diseases, and also for diseases less well-identified as inflammatory including atherosclerosis⁽²⁴⁾. There is a need, therefore, for antagonists of MIF to limit the recruitment of leukocytes into inflammatory lesions and lesions of diseases such as atherosclerosis.

In WO 03/104203, the present applicant has shown that certain benzimidazole derivatives are capable of acting as inhibitors of MIF. The present inventors have now found a novel class of MIF inhibitors, members of which show improved characteristics as drug-like molecules when compared to the compounds of the prior art.

### SUMMARY OF THE INVENTION

In a first aspect, the present invention provides a method of treating, diagnosing or preventing autoimmune diseases, tumours, or chronic or acute inflammatory diseases comprising administering a treatment, prevention or diagnostic effective amount of a compound of formula (I) or a pharmaceutically acceptable salt or prodrug thereof to a subject in need thereof wherein:

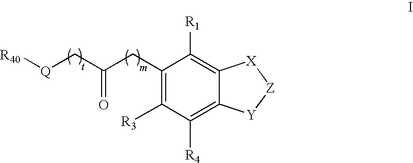

X is selected from —O—, —S—, —C(R₅)(R_{5′})— and —N(R₆)—;
Y is selected from —N(R₇)—, —O—, —S—, and —C(R₇)₂—;
R₁ is selected from hydrogen, C₁-C₃alkyl, (CR₅R_{5′})ₙOR₇, C(R₅R_{5′})ₙSR₇, (CR₅R_{5′})ₙN(R₆)₂ and (CR₅R_{5′})ₙ halo;
R₃ is selected from hydrogen, C₁-C₆alkyl, (CR₁₆R_{16′})ₚNR₁₄R₁₅, (CR₁₆R_{16′})ₚOR₁₇, (CR₁₆R_{16′})ₚSR₁₇, (CR₁₆R_{16′})ₚhalo, (CR₁₆R_{16′})ₚNO₂, (CR₁₆R_{16′})ₙC(O)R₂₈, (CR₁₆R_{16′})ₙC(═NR₂₄)R₂₂, (CR₁₆R_{16′})ₙS(O)R₁₇, (CR₁₆R_{16′})ₙS(O)₂R₁₇, (CR₁₆R_{16′})ₙS(O)₃R₁₇, and (CR₁₆R_{16′})ₚC(R₁₈)₃;
R₄ is selected from hydrogen, halogen, C₁-C₃alkyl, C₂-C₃alkenyl, C₂-C₃alkynyl and (CR₁₂R_{12′})ₙ(CR₁₈)₃;
each R₅ and R_{5′} is independently selected from hydrogen, C₁-C₃alkyl, halo, OR₇, SR₇ and N(R₆)₂;
each R₆ is independently selected from hydrogen, C₁-C₃alkyl and OR₇;
each R₇ is independently selected from hydrogen and C₁-C₃alkyl;
each R₁₂ and R_{12′} is independently selected from hydrogen, C₁-C₆alkyl, C₂-C₆alkenyl, C₂-C₆alkynyl, OR₂₄, SR₂₄, halo, N(R₂₄)_{z}, CO₂R₂₄, CN, NO₂, aryl and heterocyclyl;
each R₁₄ and R₁₅ is independently selected from hydrogen, C₁-C₃alkyl, OR₁₇, SR₁₇, and N(R₁₇)₂;
each R₁₆ and R_{16′} is independently selected from hydrogen, C₁-C₃alkyl, halo, OR₁₇, SR₁₇ and N(R₁₇)₂;
each R₁₇ is independently selected from hydrogen and C₁-C₃alkyl;
each R₁₈ is independently selected from hydrogen and halo;
R₂₂ is selected from C₁-C₆alkyl, NH₂, NH(C₁-C₆alkyl), N(C₁-C₆alkyl)₂, OR₂₉ or SR₂₉;
each R₂₄ is selected from H and C₁-C₆alkyl;
R₂₈ is selected from hydrogen, C₁-C₆alkyl, OR₂₉, SR₂₉ or N(R₂₉)₂;
each R₂₉ is independently selected from hydrogen and C₁-C₃alkyl;
Q is selected from O, S, NR₄₀, S(O)ᵤ where u is an integer from 1 to 2;
R₄₀ is selected from H, OH, and C(R₄₁R_{41′})ᵥR₄₂;
each R₄₁ and R_{41′} is independently selected from H, OH, halo, NH₂, cyano, and NO₂;
R₄₂ is independently selected from H, OR₄₃, COOR₄₃, CON(R₄₃R_{43′}), O(CO)R₄₃, aryl, and heterocyclyl;
each R₄₃ and R_{43′} is independently selected from H, C₁₋₆alkyl, benzyl, and aryl;
n=0 or an integer to 3
m is 0 or an integer from 1 to 20;
p is 0 or an integer from 1 to 6;
t is an integer from 1 to 10
v is 0 or an integer from 1 to 10.

In particular, the autoimmune disease, tumour, or chronic or acute inflammatory disease is selected from the group comprising:
rheumatic diseases (including but not limited to rheumatoid arthritis, osteoarthritis, psoriatic arthritis) spondyloarthropathies (including but not limited to ankylosing spondylitis, reactive arthritis, Reiter's syndrome), crystal arthropathies (including but not limited to gout, pseudogout, calcium pyrophosphate deposition disease), Lyme disease, polymyalgia rheumatica;
connective tissue diseases (including but not limited to systemic lupus syndrome);
vasculitides (including but not limited to polyarteritis nodosa, Wegener's granulomatosis, Churg-Strauss syndrome);
inflammatory conditions including consequences of trauma or ischaemia;
sarcoidosis;
vascular diseases including atherosclerotic vascular disease and infarction, atherosclerosis, and vascular occlusive disease (including but not limited to atherosclerosis, ischaemic heart disease, myocardial infarction, stroke, peripheral vascular disease), and vascular stent restenosis;
ocular diseases including uveitis, corneal disease, iritis, iridocyclitis, cataracts; autoimmune diseases (including but not limited to diabetes mellitus, thyroiditis, myasthenia gravis, sclerosing cholangitis, primary biliary cirrhosis);
pulmonary diseases (including but not limited to diffuse interstitial lung diseases, pneumoconioses, fibrosing alveolitis, asthma, bronchitis, bronchiectasis, chronic obstructive pulmonary disease, adult respiratory distress syndrome);
cancers whether primary or metastatic (including but not limited to prostate cancer, colon cancer, lymphoma, lung cancer, melanoma, multiple myeloma, breast cancer, stomach cancer, leukaemia, cervical cancer and metastatic cancer);
renal diseases including glomerulonephritis, interstitial nephritis;
disorders of the hypothalamic-pituitary-adrenal axis;
nervous system disorders including multiple sclerosis, Alzheimer's disease;
diseases characterised by modified angiogenesis (eg diabetic retinopathy, rheumatoid arthritis, cancer), endometrial function (menstruation, implantation.
complications of infective disorders including endotoxic (septic) shock, exotoxic (septic) shock, infective (true septic) shock, malarial complications, other complications of infection, pelvic inflammatory disease;
transplant rejection, graft-versus-host disease;
allergic diseases including allergies, atopic diseases, allergic rhinitis;
bone diseases (eg osteoporosis, Paget's disease);
skin diseases including psoriasis, atopic dermatitis, UV(B)-induced dermal cell activation (eg sunburn, skin cancer);
diabetes mellitus and its complications;
pain, testicular dysfunctions and wound healing;
gastrointestinal diseases including inflammatory bowel disease (including but not limited to ulcerative colitis, Crohn's disease), peptic ulceration, gastritis, oesophagitis, liver disease (including but not limited to cirrhosis, hepatitis).

MIF cytokine or biological activity is implicated in the above diseases and conditions.

Preferably, the disease or condition is selected from the group consisting of rheumatoid arthritis, systemic lupus erythematosus, ulcerative colitis, Crohn's disease, multiple sclerosis, psoriasis, uveitis, diabetes mellitus, glomerulonephritis, atherosclerotic vascular disease and infarction, asthma and chronic obstructive pulmonary disease.

In a second aspect, the present invention provides a compound of Formula (II) or a pharmaceutically acceptable salt or prodrug thereof wherein:

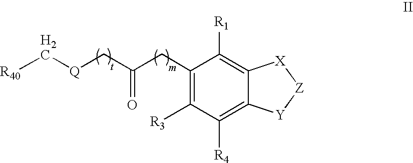

X is selected from —O—, —S—, —C(R₅)(R_{5′})— and —N(R₆)—;
Y is selected from —N(R₇)—, —O—, and —S—;
Z is selected from >C═O, >C═S, and >C═NR₆;
R₁ is selected from hydrogen, C₁-C₃alkyl, (CR₅R_{5′})ₙOR₇, C(R₅R_{5′})ₙSR₇, (CR₅R_{5′})ₙN(R₆)₂ and (CR₅R_{5′})ₙhalo;
R₃ is selected from hydrogen, C₁-C₆alkyl, (CR₁₆R_{16′})ₚNR₁₄R₁₅, (CR₁₆R_{16′})ₚOR₁₇, (CR₁₆R_{16′})ₚSR₁₇, (CR₁₆R_{16′})ₚhalo, (CR₁₆R_{16′})ₚNO₂, (CR₁₆R_{16′})ₙC(O)R_{2H}, (CR₁₆R_{16′})ₙC(═NR₂₄)R₂₂, (CR₁₆R_{16′})ₙS(O)R₁₇, (CR₁₆R_{16′})ₙS(O)₂R₁₇, (CR₁₆R_{16′})ₙS(O)₃R₁₇, and (CR₁₆R_{16′})ₚC(R₁₈)₃;
R₄ is selected from hydrogen, halogen, C₁-C₃alkyl, C₂-C₃alkenyl, C₂-C₃alkynyl and (CR₁₂R_{12′})ₙ(CR₁₈)₃;
each R₅ and R_{5′} is independently selected from hydrogen, C₁-C₃alkyl, halo, OR₇, SR₇ and N(R₆)₂;
each R₆ is independently selected from hydrogen, C₁-C₃alkyl and OR₇;
each R₇ is independently selected from hydrogen and C₁-C₃alkyl;
each R₁₂ and R_{12′} is independently selected from hydrogen, C₁-C₆alkyl, C₂-C₆alkenyl, C₂-C₆alkynyl, OR₂₄, SR₂₄, halo, N(R₂₄)₂, CO₂R₂₄, CN, NO₂, aryl and heterocyclyl;
each R₁₄ and R₁₅ is independently selected from hydrogen, C₁-C₃alkyl, OR₁₇, SR₁₇, and N(R₁₇)₂;
each R₁₆ and R_{16′} is independently selected from hydrogen, C₁-C₃alkyl, halo, OR₁₇, SR₁₇ and N(R₁₇)₂;
each R₁₇ is independently selected from hydrogen and C₁-C₃alkyl;
each R₁₈ is independently selected from hydrogen and halo;
R₂₂ is selected from C₁-C₆alkyl, NH₂, NH(C₁-C₆alkyl), N(C₁-C₆alkyl)₂, OR₂₉ or SR₂₉;
each R₂₄ is selected from H and C₁-C₆alkyl;
R₂₈ is selected from hydrogen, C₁-C₆alkyl, OR₂₉, SR₂₉ or N(R₂₉)₂;
each R₂₉ is independently selected from hydrogen and C₁-C₃alkyl;
Q is selected from O, S, S(O)ₙ where u is an integer from 1 to 2;
R₄₀ is selected from H, OH, and C(R₄₁R_{41′})ᵥR₄₂;
each R₄₁ and R_{41′} is independently selected from H, OH, halo, NH₂, CN and NO₂;
R₄₂ is selected from H, OR₄₃, COOR₄₃, CON(R₄₃R_{43′}), (O(CO)R₄₃, N(R₄₃R_{43′}), aryl, and heterocyclyl;
each R₄₃ and R_{43′} is independently selected from H, C₁₋₆ alkyl, and benzyl;
n is 0 or 1 to 3;
m is 0 or an integer from 1 to 8;
p is 0 or an integer from 1 to 6;
t is an integer from 1 to 10;
v is 0 or an integer from 1 to 10
provided that the compound is not

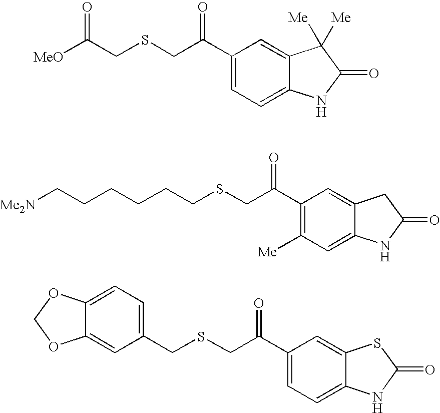

In a third aspect, the present invention provides a compound of Formula III or a pharmaceutically acceptable salt or prodrug thereof wherein:

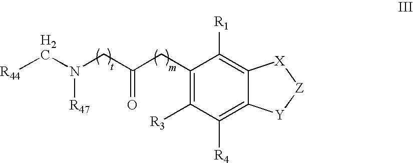

X is selected from —O—, —S—, —C(R₅)(R_{5′})— and —N(R₆)—;
Y is selected from —N(R₇), —O—, and —S—;
R₁ is selected from hydrogen, C₁-C₃alkyl, (CR₅R_{5′})ₙOR₇, C(R₅R_{5′})ₙSR₇, (CR₅R_{5′})ₙN(R₆)₂ and (CR₅R_{5′})ᵤhalo;
R₃ is selected from hydrogen, C₁-C₆alkyl, (CR₁₆R_{16′})ₚNR₁₄R₁₅, (CR₁₆R_{16′})ₚOR₁₇, (CR₁₆R_{16′})ₚSR₁₇, (CR₁₆R_{16′})ₚhalo, (CR₁₆R_{16′})ₚNO₂, (CR₁₆R_{16′})ₙC(O)R₂₈, (CR₁₆R_{16′})ₙC(═NR₂₄)R₂₂, (CR₁₆R_{16′})S(O)R₁₇, (CR₁₆R_{16′})ₙS(O)₂R₁₇, (CR₁₆R_{16′})ₙS(O)₃R₁₇, and (CR₁₆R_{16′})ₚC(R₁₈)₃;
R₄ is selected from hydrogen, halogen, C₁-C₃alkyl, C₂-C₃alkenyl, C₂-C₃alkynyl and (CR₁₂R_{12′})ₙ(CR₁₈)₃;
each R₅ and R_{5′} is independently selected from hydrogen, C₁-C₃alkyl, halo, OR₇, SR₇ and N(R₆)₂;
each R₆ is independently selected from hydrogen, C₁-C₃alkyl and OR₇;
each R₇ is independently selected from hydrogen and C₁-C₃alkyl;
each R₁₂ and R_{12′} is independently selected from hydrogen, C₁-C₆alkyl, C₂-C₆alkenyl, C₂-C₆alkynyl, OR₂₄, SR₂₄, halo, N(R₂₄)₂, CO₂R₂₄, CN, NO₂, aryl and heterocyclyl;
each R₁₄ and R₁₅ are independently selected from hydrogen, C₁-C₃alkyl, OR₁₇, SR₁₇, and N(R₁₇)₂;
each R₁₆ and R_{16′} is independently selected from hydrogen, C₁-C₃alkyl, halo, OR₁₇, SR₁₇ and N(R₁₇)₂;
each R₁₇ is independently selected from hydrogen and C₁-C₃alkyl;
each R₁₈ is independently selected from hydrogen and halo;
R₂₂ is selected from C₁-C₆alkyl, NH₂, NH(C₁-C₆alkyl), N(C₁-C₆alkyl)₂, OR₂₉ or SR₂₉;
each R₂₄ is selected from H and C₁-C₆alkyl;
R₂₈ is selected from hydrogen, C₁-C₆alkyl, OR₂₉, SR₂₉ or N(R₂₉)₂;
each R₂₉ is independently selected from hydrogen and C₁-C₃alkyl;
R₄₄ is selected from OH, C(R₄₅R_{45′})ᵥR₄₆;
each R₄₅ and R_{45′} is independently selected from H, OH, halo, NH₂, CN, NO₂;
each R₄₆ is selected from COOR₄₇, CON(R₄₇R_{47′}), O(CO)R₄₇, N(R₄₇R_{47′});
each R₄₇ and R_{47′} is independently selected from H, C₁₋₆alkyl, benzyl;
wherein when v is greater than 1, R₄₆ can be OR₄₇;
wherein when v is greater than 2, R₄₆ can be H;
n is 0 or 1 to 3;
m is 0 or an integer from 1 to 8;
p is 0 or an integer from 1 to 6;
t is an integer from 1 to 10;
v is 0 or an integer from 1 to 10
provided that the compound is not

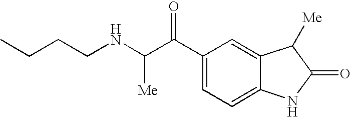

A further aspect of the invention provides for the use of a compound of Formula (I) or a pharmaceutically acceptable salt or prodrug thereof in the manufacture of a medicament for the treatment of a disease or condition as above.

A further aspect of the invention provides a pharmaceutical composition comprising a compound of the second or third aspect and a pharmaceutically acceptable carrier, diluent or excipient.

In a further aspect, the present invention provides a method of inhibiting cytokine or biological activity of MIF comprising contacting MIF with a cytokine or biological inhibiting amount of a compound of formula (I), or a pharmaceutically acceptable salt or prodrug thereof.

In another aspect, the invention provides a method of treating, preventing or diagnosing a disease or condition wherein MIF cytokine or biological activity is implicated comprising the administration of a treatment, prevention or diagnostic effective amount of a compound of formula (I) or a pharmaceutically acceptable salt or prodrug thereof to a subject in need thereof.

In a further aspect there is provided the use of a compound of formula (I) or a pharmaceutically acceptable salt or prodrug thereof in the manufacture of a medicament for the treatment, prevention or diagnosis of a disease or condition wherein MIF cytokine or biological activity is implicated.

In another aspect, the invention provides a method of treating or preventing a disease or condition wherein MIF cytokine or biological activity is implicated comprising:
administering to a mammal a compound of formula (I) and a second therapeutic agent.

In another aspect, the present invention provides a method of prophylaxis or treatment of a disease or condition for which treatment with a glucocorticoid is indicated, said method comprising:
administering to a mammal a glucocorticoid and a compound of formula (I).

In yet another aspect, the present invention provides a method of treating steroid-resistant
administering to a mammal a glucocorticoid and a compound of formula (I).

In a further aspect, the present invention provides a method of enhancing the effect of a glucocorticoid in mammals comprising administering a compound of formula (I) simultaneously, separately or sequentially with said glucocorticoid.

In yet a further aspect, the present invention provides a pharmaceutical composition comprising a glucocorticoid and a compound of formula (I).

In a further aspect of the invention there is provided a use of a glucocorticoid in the manufacture of a medicament, for administration with a compound of formula (I) for the treatment or prophylaxis of a disease or condition for which treatment with a glucocorticoid is indicated.

In yet a further aspect of the invention there is provided a use of a compound of formula (I) in the manufacture of a medicament for administration with a glucocorticoid for the treatment or prophylaxis of a disease or condition for which treatment of a glucocorticoid is indicated.

In yet a further aspect of the invention there is provided a use of a glucocorticoid and a compound of formula (I) in the manufacture of a medicament for the treatment or prophylaxis of a disease or condition for which treatment with a glucocorticoid is indicated.

Inhibitors of MIF may also be used in implantable devices such as stents. Accordingly, in a further aspect the present invention provides an implantable device, preferably a stent, comprising:
(i) a reservoir containing at least one compound of formula (I); and
(ii) means to release or elute the inhibitor from the reservoir

There is further provided a method for inhibiting the cytokine or biological activity of MIF in a subject comprising the step of implanting an implantable device according to the invention in the subject.

In a yet further aspect, the present invention provides a method of treating, preventing or diagnosing a disease or condition wherein MIF cytokine activity is implicated comprising the step of implanting an implantable device according to the invention in a subject in need thereof.

The present invention further provides an angioplastic stent for inhibiting the onset of restenosis, which comprises an angioplasty stent operably coated with a prophylactically effective dose of a composition comprising at least one compound of formula (I).

The present invention further provides a method for inhibiting the onset of restenosis in a subject undergoing angioplasty, which comprises topically administering a stent according to the present invention to the subject at around the time of the angioplasty.

There is further provided a method of reducing the severity of stent restenosis in the vicinity of a stent comprising die use of a stent according to the present invention.

### BRIEF DESCRIPTION OF THE FIGURES

FIG. 1 shows that treatment with a compound according to the present invention induces a dose-dependent inhibition of LPS-induced IL-6 production in a mouse macrophage cell line.

FIG. 2 shows that treatment with a compound according to the present invention induces a dose-dependent inhibition of IL-1 induced COX-2 expression when S112 cells are treated with up to 100 μM concentration of compound.

FIG. 3A shows that treatment of mice with compound 15 according to the present invention results in a significant dose-dependent suppression of LPS-induced serum TNF levels in a mouse model of endotoxic shock.

FIG. 3B shows that treatment of mice with compounds 2 and 13 according to die present invention results in a significant dose-dependent suppression of UPS-induced serum TNF levels in a mouse model of endotoxic shock.

FIG. 3C shows that treatment of mice with compound 4 according to the present invention results in a significant dose-dependent suppression of UPS-induced serum TNF levels in a mouse model of endotoxic shock.

FIG. 3D shows that treatment of mice with compound 19 according to the present invention results in a significant dose-dependent suppression of LPS-induced scrum TNF levels in a mouse model of endotoxic shock.

FIG. 4 shows reduction in DTH reactions in vivo in mice treated with compound 13.

FIG. 5 shows effect of compound 13 on rhMIF-induced leukocyte adhesion.

### DETAILED DESCRIPTION OF THE PREFERRED EMBODIMENTS

In a first aspect, the present invention provides a method of treating, diagnosing or preventing autoimmune diseases, tumours, or chronic or acute inflammatory diseases comprising administering a treatment, prevention or diagnostic effective amount of a compound of formula (I) or a pharmaceutically acceptable salt or prodrug thereof to a subject in need thereof wherein:

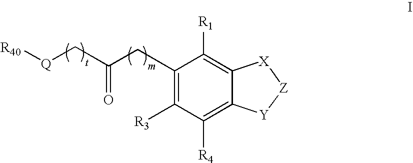

X is selected from —O—, —S—, —C(R₅)(R_{5′})— and —N(R₆)—;
Y is selected from —N(R₇)—, —O—, —S—, and —C(R₇)₂—;
Z is selected from >C═O, >C═S, >C—NR₆, >S═O and >S(O)₂;
R₁ is selected from hydrogen, C₁-C₃alkyl, (CR₅R_{5′})ₙOR₇, C(R₅R_{5′})ₙSR₇, (CR₅R_{5′})ₙN(R₆)₂ and (CR₅R_{5′})ₙhalo;
R₃ is selected from hydrogen, C₁-C₆alkyl, (CR₁₆R_{16′})ₚNR₁₄R₁₅, (CR₁₆R_{16′})ₚOR₁₇, (CR₁₆R_{16′})ₚSR₁₇, (CR₁₆R_{16′})ₚhalo, (CR₁₆R_{16′})ₚNO₂, (CR₁₆R_{16′})ₙC(O)R₂₈, (CR₁₆R_{16′})ₙC(═NR₂₄)R₂₂, (CR₁₆R_{16′})ₙS(O)R₁₇, (CR₁₆R_{16′})ₙS(O)₂R₁₇, (CR₁₆R_{16′})ₙS(O)₃R₁₇, and (CR₁₆R_{16′})ₚC(R₁₈)₃;
R₄ is selected from hydrogen, halogen, C₁-C₃alkyl, C₂-C₃alkenyl, C₂-C₃alkynyl and (CR₁₂R_{12′})ₙ(CR₁₈)₃;
each R₅ and R_{5′} is independently selected from hydrogen, C₁-C₃alkyl, halo, OR₇, SR₇ and N(R₆)₂;
each R₆ is independently selected from hydrogen, C₁-C₃alkyl and OR₇;
each R₇ is independently selected from hydrogen and C₁-C₃alkyl;
each R₁₂ and R_{12′} is independently selected from hydrogen, C₁-C₆alkyl, C₂-C₆alkenyl, C₂-C₆alkynyl, OR₂₄, SR₂₄, halo, N(R₂₄)₂, CO₂R₂₄, CN, NO₂, aryl and heterocyclyl;
each R₁₄ and R₁₅ is independently selected from hydrogen, C₁-C₃alkyl, OR₁₇, SR₁₇, and N(R₁₇)₂;
each R₁₆ and R_{16′} is independently selected from hydrogen, C₁-C₃alkyl, halo, OR₁₇, SR₁₇ and N(R₁₇)₂;
each R₁₇ is independently selected from hydrogen and C₁-C₃alkyl;
each R₁₈ is independently selected from hydrogen and halo;
R₂₂ is selected from C₁-C₆alkyl, NH₂, NH(C₁-C₆alkyl), N(C₁-C₆alkyl)₂, OR₂₉ or SR₂₉;
each R₂₄ is selected from H and C₁-C₆alkyl;
R₂₈ is selected from hydrogen, C₁-C₆alkyl, OR₂₉, SR₂₉ or N(R₂₉)₂;
each R₂₉ is independently selected from hydrogen and C₁-C₃alkyl;
Q is selected from O, S, NR₄₀, S(O)ᵤ where u is an integer from 1 to 2;
R₄₀ is selected from H, OH, and C(R₄₁R_{41′})ᵥR₄₂;
each R₄₁ and R_{41′} is independently selected from H, OH, halo, NH₂, cyano, and NO₂;
R₄₂ is independently selected from H, OR₄₃, COOR₄₃, CON(R₄₃R_{43′}), O(CO)R₄₃, aryl, and heterocyclyl;
each R₄₃ and R_{43′} is independently selected from H, C₁₋₆alkyl, benzyl, and aryl;
n=0 or an integer to 3
m is 0 or an integer from 1 to 20;
p is 0 or an integer from 1 to 6;
t is an integer from 1 to 10
v is 0 or an integer from 1 to 10.

In particular, the autoimmune disease, tumour, or chronic or acute inflammatory disease is selected from the group comprising:
rheumatic diseases (including but not limited to rheumatoid arthritis, osteoarthritis, psoriatic arthritis) spondyloarthropathies (including but not limited to ankylosing spondylitis, reactive arthritis, Reiter's syndrome), crystal arthropathies (including but not limited to gout, pseudogout, calcium pyrophosphate deposition disease), Lyme disease, polymyalgia rheumatica; connective tissue diseases (including but not limited to systemic lupus erythematosus, systemic sclerosis, polymyositis, dermatomyositis, Sjögren's syndrome);
vasculitides (including but not limited to polyarteritis nodosa, Wegener's granulomatosis, Churg-Strauss syndrome);
inflammatory conditions including consequences of trauma or ischaemia; sarcoidosis;
vascular diseases including atherosclerotic vascular disease and infarction, atherosclerosis, and vascular occlusive disease (including but not limited to atherosclerosis, ischaemic heart disease, myocardial infarction, stroke, peripheral vascular disease), and vascular stent restenosis;
ocular diseases including uveitis, corneal disease, iritis, iridocyclitis, cataracts; autoimmune diseases (including but not limited to diabetes mellitus, thyroiditis, myasthenia gravis, sclerosing cholangitis, primary biliary cirrhosis);
pulmonary diseases (including but not limited to diffuse interstitial lung diseases, pneumoconioses, fibrosing alveolitis, asthma, bronchitis, bronchiectasis, chronic obstructive pulmonary disease, adult respiratory distress syndrome);
cancers whether primary or metastatic (including but not limited to prostate cancer, colon cancer, lymphoma, lung cancer, melanoma, multiple myeloma, breast cancer, stomach cancer, leukaemia, cervical cancer and metastatic cancer);
renal diseases including glomerulonephritis, interstitial nephritis;
disorders of the hypothalamic-pituitary-adrenal axis;
nervous system disorders including multiple sclerosis, Alzheimer's disease; rheumatoid arthritis, cancer), endometrial function (menstruation, implantation, endometriosis);
complications of infective disorders including endotoxic (septic) shock, exotoxic (septic) shock, infective (true septic) shock, malarial complications, other complications of infection, pelvic inflammatory disease;
transplant rejection, graft-versus-host disease;
allergic diseases including allergies, atopic diseases, allergic rhinitis;
bone diseases (eg osteoporosis, Paget's disease);
skin diseases including psoriasis, atopic dermatitis, UV(B)-induced dermal cell activation (eg sunburn, skin cancer);
diabetes mellitus and its complications;
pain, testicular dysfunctions and wound healing;
gastrointestinal diseases including inflammatory bowel disease (including but not limited to ulcerative colitis, Crohn's disease), peptic ulceration, gastritis, oesophagitis, liver disease (including but not limited to cirrhosis, hepatitis).

MIF cytokine or biological activity is implicated in the above diseases and conditions.

Preferably, the disease or condition is selected from the group consisting of rheumatoid arthritis, systemic lupus erythematosus, ulcerative colitis, Crohn's disease, multiple sclerosis, psoriasis, uveitis, diabetes mellitus, glomerulonephritis, atherosclerotic vascular disease and infarction, asthma and chronic obstructive pulmonary disease.

In a preferred form Q is S.

In a further preferred form, R₄₀ is C(R₄₁R_{41′})ᵥR₄₂ wherein R₄₂ is COOR₄₃. More preferably, R₄₃ is hydrogen or C₁-C₆alkyl, preferably methyl.

In another preferred form, the compound of Formula I is selected from any one of Compounds 1 to 32 as set out in the Examples herein.

Particularly preferred are Compounds 2, 13 and 19.

As used herein, the term “effective amount” relates to an amount of compound which, when administered according to a desired dosing regimen, provides the desired MIF cytokine inhibiting or treatment or therapeutic activity, or disease/condition prevention. Dosing may occur at intervals of minutes, hours, days, weeks, months or years or continuously over any one of these periods. A cytokine or biological activity inhibiting amount is an amount which will at least partially inhibit the cytokine or biological activity of MIF. A therapeutic, or treatment, effective amount is an amount, of the compound which, when administered according to a desired dosing regimen, is sufficient to at least partially attain the desired therapeutic effect, or delay the onset of, or inhibit the progression of or halt or partially or fully reverse the onset or progression of a particular disease condition being treated. A prevention effective amount is an amount of compound which when administered according to the desired dosing regimen is sufficient to at least partially prevent or delay the onset of a particular disease or condition. A diagnostic effective amount of compound is an amount sufficient to bind to MIF to enable detection of the MIF-compound complex such that diagnosis of a disease or condition is possible.

Suitable dosages may lie within the range of about 0.1 ng per kg of body weight to 1 g per kg of body weight per dosage. The dosage is preferably in the range of 1 μg to 1 g per kg of body weight per dosage, such as is in the range of 1 mg to 1 g per kg of body weight per dosage. In one embodiment, the dosage is in the range of 1 mg to 500 mg per kg of body weight per dosage. In another embodiment, die dosage is in the range of 1 mg to 250 mg per kg of body weight per dosage. In yet another preferred embodiment, the dosage is in the range of 1 mg to 100 mg per kg of body weight per dosage, such as up to 50 mg per kg of body weight per dosage. In yet another embodiment, the dosage is in the range of 1 μg to 1 mg per kg of body weight per dosage.

Suitable dosage amounts and dosing regimens can be determined by the attending physician or veterinarian and may depend on the desired level of inhibiting activity, the particular condition being treated, the severity of the condition as well as the general age, health and weight of the subject.

The active ingredient may be administered in a single dose or a series of doses. While it is possible for the active ingredient to be administered alone, it is preferable to present it as a composition, preferably as a pharmaceutical composition.

It will be recognised that other therapeutically active agents such as anti-inflammatory (eg steroids such as glucocorticoids) or anti-cancer agents may be used in conjunction with a compound of Formula (I). Compounds of Formula (I) when administered in conjunction with other therapeutically active agents may exhibit an additive or synergistic effect. These may be administered simultaneously, either as a combined form (ie as a single composition containing the active agents) or as discrete dosages. Alternatively, the other therapeutically active agents may be administered sequentially or separately with the compounds of the invention. Thus, the invention also relates to kits and combinations, comprising a compound of Formula (I) and one or more other therapeutically active ingredients for use in the treatment of diseases or conditions described herein. Without being limiting, examples of agents which could be used in combination with a compound of Formula (I) include: antirheumatic drugs (including but not limited to methotrexate, leflunomide, sulphasalazine, hydroxycholorquine, gold salts); immunosuppressive drugs (including but not limited to cyclosporin, mycophenyllate mofetil, azathioprine, cyclophosphamide); anti-cytokine therapies (including but not limited to antagonists of, antibodies to, binding proteins for, or soluble receptors for tumor necrosis factor, interleukin 1, interleukin 3, interleukin 5, interleukin 6, interleukin 8, interleukin 12, interleukin 18, interleukin 17, and other pro-inflammatory cytokines as may be found relevant to pathological states); antagonists or inhibitors of mitogen-activated protein (MAP) kinases (including but not limited to antagonists or inhibitors of extracellular signal-regulated kinases (ERK), the c-Jun N-terminal kinases/stress-activated protein kinases (JNK/SAPK), and the p38 MAP kinases, and other kinases or enzymes or proteins involved in MAP kinase-dependent cell activation); antagonists or inhibitors of the nuclear factor kappa-B (NF-κB) signal transduction pathway (including but not limited to antagonists or inhibitors of 1-κB-kinase, interleukin receptor activated kinase, and other kinases or enzymes or proteins involved in NF-κB-dependent cell activation); antibodies, protein therapeutics, or small molecule therapeutics interacting with adhesion molecules and co-stimulatory molecules (including but not limited to therapeutic agents directed against intercellular adhesion molecule-1, CD40, CD40-ligand, CD28, CD4, CD-3, selectins such as P-selectin or E-selectin); bronchodilators such as (3-adrenoceptor agonists or anti-cholinergics; antagonists of eicosanoid synthesis pathways such as non-steroidal anti-inflammatory drugs, cyclooxygenase-2 inhibitors, thromboxane inhibitors, or lipoxygenase inhibitors; antibodies or other agents directed against leukocyte surface antigens (including but not limited to antibodies or other agents directed against CD3, CD4, CD5, CD19, CD20, HLA molecules, BLyS); agents used for the treatment of inflammatory bowel disease (including but not limited to sulphasalazine, mesalazine, salicylic acid derivatives); anti-cancer drugs (including but not limited to cytotoxic drugs, cytolytic drugs, monoclonal antibodies).

Accordingly, preferably, the compound of formula (I) is administered in conjunction with a second therapeutic agent. More preferably, the second therapeutic agent is a glucocorticoid.

Preferably, the compound of Formula (I) is a compound of Formula (II) wherein:

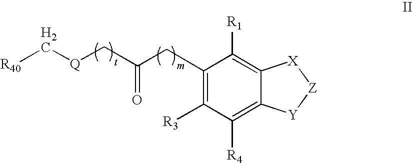

X is selected from —O—, —S—, —C(R₅)(R_{5′})— and —N(R₆)—;
Y is selected from —N(R₇)—, —O—, and —S—;
Z is selected from >C═O, >C═S, and >C═NR₆;
R₁ is selected from hydrogen, C₁-C₃alkyl, (CR₅R_{5′})ₙOR₇, C(R₅R_{5′})ₙSR₇, (CR₅R_{5′})ₙN(R₆)₂ and (CR₅R_{5′})ₙhalo;
R₃ is selected from hydrogen, C₁-C₆alkyl, (CR₁₆R_{16′})ₚNR₁₄R₁₅, (CR₁₆R_{16′})ₚOR₁₇, (CR₁₆R_{16′})ₚSR₁₇, (CR₁₆R_{16′})ₚhalo, (CR₁₆R_{16′})ₚNO₂, (CR₁₆R_{16′})ₙC(O)R₂₈, (CR₁₆R_{16′})ₙC(═NR₂₄)R₂₂, (CR₁₆R_{16′})ₙS(O)R₁₇, (CR₁₆R_{16′})ₙS(O)₂R₁₇, (CR₁₆R_{16′})ₙS(O)₃R₁₇, and (CR₁₆R_{16′})ₚC(R₁₈)₃;
R₄ is selected from hydrogen, halogen, C₁-C₃alkyl, C₂-C₃alkenyl, C₂-C₃alkynyl and (CR₁₂R_{12′})ₙ(CR₁₈)₃;
each R₅ and R_{5′} is independently selected from hydrogen, C₁-C₃alkyl, halo, or OR₇, SR₇ and N(R₆)₂;
each R₆ is independently selected from hydrogen, C₁-C₃alkyl and OR₇;
each R₇ is independently selected from hydrogen and C₁-C₃alkyl;
each R₁₂ and R_{12′} is independently selected from hydrogen, C₁-C₆alkyl, C₂-C₆alkenyl, C₂-C₆alkynyl, OR₂₄, SR₂₄, halo, N(R₂₄)₂, CO₂R₂₄, CN, NO₂, aryl and heterocyclyl;
each R₁₄ and R₁₅ is independently selected from hydrogen, C₁-C₃alkyl, OR₁₇, SR₁₇, and N(R₁₇)₂;
each R₁₆ and R_{16′} is independently selected from hydrogen, C₁-C₃alkyl, halo, OR₁₇, SR₁₇ and N(R₁₇)₂;
each R₁₇ is independently selected from hydrogen and C₁-C₃alkyl;
each R₁₈ is independently selected from hydrogen and halo;
R₂₂ is selected from C₁-C₆alkyl, NH₂, NH(C₁-C₆alkyl), N(C₁-C₆alkyl)₂, OR₂₉ or SR₂₉;
each R₂₄ is selected from H and C₁-C₆alkyl;
R₂₈ is selected from hydrogen, C₁-C₆alkyl, OR₂r>, SR₂₉ or N(R₂₉)₂;
each R₂₉ is independently selected from hydrogen and C₁-C₃alkyl;
Q is selected from O, S, S(O)ᵤ where u is an integer from 1 to 2;
R₄₀ is selected from H, OH, and C(R₄₁R_{41′})ᵥR₄₂;
each R₄₁ and R_{41′} is independently selected from H, OH, halo, NH₂, CN and NO₂;
R₄₂ is selected from H, OR₄₃, COOR₄₃, CON(R₄₃R_{43′}), O(CO)R₄₃, N(R₄₃R_{43′}), aryl, and heterocyclyl;
each R₄₃ and R_{43′} is independently selected from H, C₁₋₆alkyl, and benzyl;
n is 0 or 1 to 3;
m is 0 or an integer from 1 to 8;
p is 0 or an integer from 1 to 6;
t is an integer from 1 to 10;
v is 0 or an integer from 1 to 10.

Preferably, the compound of Formula (I) is a compound of Formula (III) wherein:

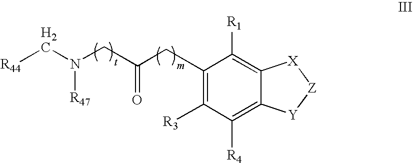

X is selected from —O—, —S—, —C(R₅)(R_{5′})— and —N(R₆)—;
Y is selected from —N(R₇)—, —O—, and —S—;
Z is selected from >C═O, >C═S, and >C═NR₆;
R₁ is selected from hydrogen, C₁-C₃alkyl, (CR₅R_{5′})ₙOR₇, C(R₅R_{5′})ₙSR₇, (CR₅R_{5′})ₙN(R₆)₂ and (CR₅R_{5′})ₙhalo;
R₃ is selected from hydrogen, C₁-C₆alkyl, (CR₁₆R_{16′})ₚNR₁₄R₁₅, (CR₁₆R_{16′})ₚOR₁₇, (CR₁₆R_{16′})ₚSR₁₇, (CR₁₆R_{16′})ₚhalo, (C₁₆R_{16′})ₚNO₂, (CR₁₆R_{16′})ₙC(O)R₂₈, (CR₁₆R_{16′})ₙC(═NR₂₄)R₂₂, (CR₁₆R_{16′})S(O)R₁₇, (CR₁₆R_{16′})ₙS(O)₂R₁₇, (CR₁₆R_{16′})ₙS(O)₃R₁₇, and (CR₁₆R_{16′})ₚC(R₁₈)₃;
R₄ is selected from hydrogen, halogen, C₁-C₃alkyl, C₂-C₃alkenyl, C₂-C₃alkynyl and (CR₁₂R_{12′})ₙ(CR₁₈)₃;
each R₅ and R_{5′} is independently selected from hydrogen, C₁-C₃alkyl, halo, OR₇, SR₇ and N(R₆)₂;
each R₆ is independently selected from hydrogen, C₁-C₃alkyl and OR₇;
each R₇ is independently selected from hydrogen and C₁-C₃alkyl;
each R₁₂ and R_{12′} is independently selected from hydrogen, C₁-C₆alkyl, C₂-C₆alkenyl, C₂-C₆alkynyl, OR₂₄, SR₂₄, halo, N(R₂₄)₂, CO₂R₂₄, CN, NO₂, aryl and heterocyclyl;
each R₁₄ and R₁₅ are independently selected from hydrogen, C₁-C₃alkyl, OR₁₇, SR₁₇, and N(R₁₇)₂;
each R₁₆ and R_{16′} is independently selected from hydrogen, C₁-C₃alkyl, halo, OR₁₇, SR₁₇ and N(R₁₇)₂;
each R₁₇ is independently selected from hydrogen and C₁-C₃alkyl;
each R₁₈ is independently selected from hydrogen and halo;
R₂₂ is selected from C₁-C₆alkyl, NH₂, NH(C₁-C₆alkyl), N(C₁-C₆alkyl)₂, OR₂₉ or SR₂₉;
each R₂₄ is selected from H and C₁-C₆alkyl;
R₂₈ is selected from hydrogen, C₁-C₆alkyl, OR₂₉, SR₂₉ or N(R₂₉)₂;
each R₂₉ is independently selected from hydrogen and C₁-C₃alkyl;
R₄₄ is selected from OH, C(R₄₅R_{45′})ᵥR₄₆;
each R₄₅ and R_{45′} is independently selected from H, OH, halo, NH₂, CN, NO₂;
each R₄₆ is selected from COOR₄₇, CON(R₄₇R_{47′}), O(CO)R₄₇, N(R₄₇R_{47′});
each R₄₇ and R_{47′} is independently selected from H, C₁₋₆ alkyl, benzyl;
wherein when v is greater than 1, R₄₆ can be OR₄₇;
wherein when v is greater than 1, R₄₆ can be H;
n is 0 or 1 to 3;
m is 0 or an integer from 1 to 8;
p is 0 or an integer from 1 to 6;
t is an integer from Y to 10;
v is 0 or an integer from 1 to 10.

In a second aspect, the present invention provides a compound of Formula (II) or a pharmaceutically acceptable suit or prodrug thereof wherein:

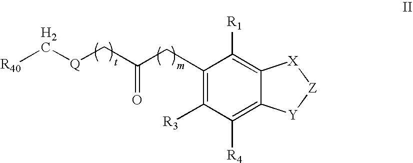

X is selected from —O—, —S—, —C(R₅)(R_{5′})— and —N(R₆)—;
Y is selected from —N(R₇)—, —O—, and —S—;
Z is selected from >C═O, >C═S, and >C═NR₆;
R₁ is selected from hydrogen, C₁-C₃alkyl, (CR₅R_{5′})ₙOR₇, C(R₅R₅)ₙSR₇, (CR₅R_{5′})ₙN(R₆)₂ and (CR₅R_{5′})ₙhalo;
R₃ is selected from hydrogen, C₁-C₆alkyl, (CR₁₆R_{16′})ₚNR₁₄R₁₅, (CR₁₆R_{16′})ₚOR₁₇, (CR₁₆R_{16′})pSR₁₇, (CR₁₆R_{16′})ₚhalo, (CR₁₆R_{16′})ₚNO₂, (CR₁₆R_{16′})ₙC(O)R₂₈, (CR₁₆R_{16′})ₙC(═NR₂₄)R₂₂, (CR₁₆R_{16′})ₙS(O)R₁₇, (CR₁₆R_{16′})ₙS(O)₂R₁₇, (CR₁₆R_{16′})ₙS(O)₃R₁₇, and (CR₁₆R_{16′})ₚC(R₁₈)₃;
R₄ is selected from hydrogen, halogen, C₁-C₃alkyl, C₂-C₃alkenyl, C₂-C₃alkynyl and (CR₁₂R_{12′})ₙ(CR₁₈)₃;
each R₅ and R_{5′} is independently selected from hydrogen, C₁-C₃alkyl, halo, OR₇, SR₇ and N(R₆)₂;
each R₆ is independently selected from hydrogen, C₁-C₃alkyl and OR₇;
each R₇ is independently selected from hydrogen and C₁-C₃alkyl;
each R₁₂ and R_{12′} is independently selected from hydrogen, C₁-C₆alkyl, C₂-C₆alkenyl, C₂-C₆alkynyl, OR₂₄, SR₂₄, halo, N(R₂₄)₂, CO₂R₂₄, CN, NO₂, aryl and heterocyclyl;
each R₁₄ and R₁₅ is independently selected from hydrogen, C₁-C₃alkyl, or OR₁₇, SR₁₇, and N(R₁₇)₂;
each R₁₆ and R_{16′} is independently selected from hydrogen, C₁-C₃alkyl, halo, OR₁₇, SR₁₇ and N(R₁₇)₂;
each R₁₇ is independently selected from hydrogen and C₁-C₃alkyl;
each R₁₈ is independently selected from hydrogen and halo;
R₂₂ is selected from C₁-C₆alkyl, NH₂, NH(C₁-C₆alkyl), N(C₁-C₆alkyl)₂, OR₂₉ or SR₂₉;
each R₂₄ is selected from H and C₁-C₆alkyl;
R₂₈ is selected from hydrogen, C₁-C₆alkyl, OR₂₉, SR₂₉ or N(R₂₉)₂,
each R₂₉ is independently selected from hydrogen and C₁-C₃alkyl;
Q is selected from O, S, S(O)ᵤ where u is an integer from 1 to 2;
R₄₀ is selected from H, OH, and C(R₄₁R_{41′})ᵥR₄₂;
each R₄₁ and R_{41′} is independently selected from H, OH, halo, NH₂, CN and NO₂;
R₄₂ is selected from H, OR₄₃, COOR₄₃, CON(R₄₃R_{43′}), O(CO)R₄₃, N(R₄₃R_{43′}), aryl, and heterocyclyl;
each R₄₃ and R_{43′} is independently selected from H, C₁₋₆ alkyl, and benzyl;
n is 0 or 1 to 3;
m is 0 or an integer from 1 to 8;
p is 0 or an integer from 1 to 6;
t is an integer from 1 to 10;
v is 0 or an integer from 1 to 10
provided that the compound is not

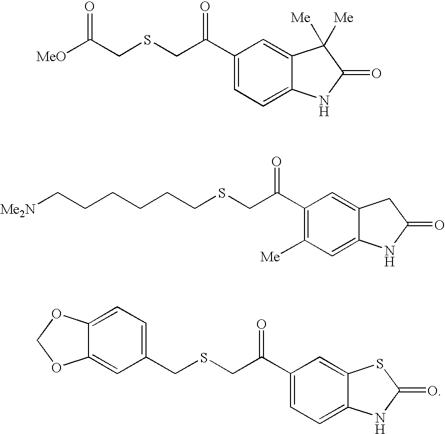

In a third aspect, the present invention provides a compound of Formula III or a pharmaceutically acceptable salt or prodrug thereof wherein:

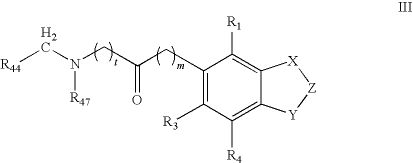

X is selected from —O—, —S—, —C(R₅)(R_{5′})— and —N(R₆)—;
Y is selected from —N(R₇)—, —O—, and —S—;
R₁ is selected from hydrogen, C₁-C₃alkyl, (CR₅R_{5′})ₙOR₇, C(R₅R_{5′})ₙSR₇, (CR₅R_{5′})ₙN(R₆)₂ and (CR₅R_{5′})ₙhalo;
R₃ is selected from hydrogen, C₁-C₆alkyl, (CR₁₆R_{16′})ₚNR₁₄R₁₅, (CR₁₆R_{16′})ₚOR₁₇, (CR₁₆R_{16′})ₚSR₁₇, (CR₁₆R_{16′})ₚhalo, (CR₁₆R_{16′})ₚNO₂, (CR₁₆R_{16′})ₙC(O)R₂₈, (CR₁₆R_{16′})ₙC(═NR₂₄)R₂₂, (CR₁₆R_{16′})S(O)R₁₇, (CR₁₆R_{16′})ₙS(O)₂R₁₇, (CR₁₆R_{16′})ₙS(O)₃R₁₇, and (CR₁₆R_{16′})ₚC(R₁₈)₃;
R₄ is selected from hydrogen, halogen, C₁-C₃alkyl, C₂-C₃alkenyl, C₂-C₃alkynyl and (CR₁₂R_{12′})ₙ(CR₁₈)₃;
each R₅ and R_{5′} is independently selected from hydrogen, C₁-C₃alkyl, halo, OR₇, SR₇ and N(R₆)₂;
each R₆ is independently selected from hydrogen, C₁-C₃alkyl and OR₇;
each R₇ is independently selected from hydrogen and C₁-C₃alkyl;
each R₁₂ and R_{12′} is independently selected from hydrogen, C₁-C₆alkyl, C₂-C₆alkenyl, C₂-C₆alkynyl, OR₂₄, SR₂₄, halo, N(R₂₄)₂, CO₂R₂₄, CN, NO₂, aryl and heterocyclyl;
each R₁₄ and R₁₅ are independently selected from hydrogen, C₁-C₃alkyl, OR₁₇, SR₁₇, and N(R₁₇)₂;
each R₁₆ and R_{16′} is independently selected from hydrogen, C₁-C₃alkyl, halo, OR₁₇, SR₁₇ and N(R₁₇)₂;
each R₁₇ is independently selected from hydrogen and C₁-C₃alkyl;
each R₁₈ is independently selected from hydrogen and halo;
R₂₂ is selected from C₁-C₆alkyl, NH₂, NH(C₁-C₆alkyl), N(C₁-C₆alkyl)₂, OR₂₉ or SR₂₉;
each R₂₄ is selected from H and C₁-C₆alkyl;
R₂₈ is selected from hydrogen, C₁-C₆alkyl, OR₂₉, SR₂₉ or N(R₂₉)₂;
each R₂₉ is independently selected from hydrogen and C₁-C₃alkyl;
R₄₄ is selected from OH, C(R₄₅R_{45′})ᵥR₄₆;
each R₄₅ and R_{45′} is independently selected from H, OH, halo, NH₂, CN, NO₂;
each R₄₆ is selected from COOR₄₇, CON(R₄₇R_{47′}), O(CO)R₄₇, N(R₄₇R_{47′});
each R₄₇ and R_{47′} is independently selected from H, C₁₋₆ alkyl, benzyl;
wherein when v is greater than 1, R₄₆ can be OR₄₇;
wherein when v is greater than 1, R₄₆ can be H;
n is 0 or 1 to 3;
m is 0 or an integer from 1 to 8;
p is 0 or an integer from 1 to 6;
t is an integer from 1 to 10;
v is 0 or an integer from 1 to 10
provided that the compound is not

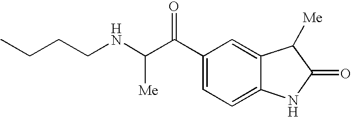

As used herein, the term “alkyl” refers to monovalent straight, branched or, where appropriate, cyclic aliphatic radicals, having 1 to 3, 1 to 6, 1 to 10 or 1 to 20 carbon atoms, e.g. methyl, ethyl, n-propyl, iso-propyl, cyclopropyl, n-butyl, sec-butyl, t-butyl and cyclobutyl, n-pentyl, 1-methylbutyl, 2methylbutyl, 3-methylbutyl, cyclopentyl, n-hexyl, 1- 2- 3- or 4-methylpentyl, 1- 2- or 3-ethylbutyl, 1 or 2-propylpropyl or cyclohexyl.

An alkyl group may be optionally substituted one or more times by halo (eg chloro, fluoro or bromo), CN, NO₂, CO₂H, CO₂C₁₋₆alkyl, CO₂NH₂, CO₂NH(C₁₋₆alkyl), CO₂N(C₁₋₆alkyl)₂, OH, alkoxy, acyl, acetyl, halomethyl, trifluoromethyl, benzyloxy, phenoxy, NH₂, NH(C₁₋₆alkyl) or N(C₁₋₆alkyl)₂. A preferred optional substituent is a polar substituent. Examples of alkoxy include methoxy, ethoxy, n-propoxy, iso-propoxy, cyclopropoxy, and butoxy (n-, sec- t- and cyclo) pentoxy and hexyloxy. The “alkyl” portion of an alkoxy group may be substituted as described above.

As used herein, the term “alkenyl” refers to straight, branched, or where appropriate, cyclic carbon containing radicals having one or more double bonds between carbon atoms. Examples of such radicals include vinyl, allyl, butenyl, or longer carbon chains such as those derived from palmitoleic, oleic, linoleic, linolenic or arachidonic acids. An alkenyl group may be optionally substituted one or more times by halo (eg chloro, fluoro or bromo), CN, NO₂, CO₂H, CO₂C₁₋₆alkyl, CO₂NH₂, CO₂NH(C₁₋₆alkyl), CO₂N(C₁₋₆alkyl)₂, OH, alkoxy, acyl, acetyl, halomethyl, trifluoromethyl, benzyloxy, phenoxy, NH₂, NH(C₁₋₆alkyl) or N(C₁₋₆alkyl)₂. A preferred optional substituent is a polar substituent.

As used herein, the term “alkynyl” refers to straight or branched carbon containing radicals having one or more triple bonds between carbon atoms. Examples of such radicals include propargyl, butynyl and hexynyl. An alkynyl group may be optionally substituted one or more times by halo (eg chloro, fluoro or bromo), CN, NO₂, CO₂H, CO₂C₁₋₆alkyl, CO₂NH₂, CO₂NH(C₁₋₆alkyl), CO₂N(C₁₋₆alkyl)₂, OH, alkoxy, acyl, acetyl, halomethyl, trifluoromethyl, benzyloxy, phenoxy, NH₂, NH(C₁₋₆alkyl) or N(C₁₋₆alkyl)₂. A preferred optional substituent is a polar substituent.

Examples of suitable NH(alkyl) and N(alkyl)₂ include methylamino, ethylamino, isopropylamino, dimethylamino, n-propylamino, diethylamino and di-isopropylamino.

The term “halogen” (or “halo”) refers to fluorine (fluoro), chlorine (chloro), bromine (bromo)

An aryl group, as used herein, refers to C₆-C₁₀ aryl groups such as phenyl or naphthalene. Aryl groups may be optionally substituted one or more times by halo (eg, chloro, fluoro or bromo), CN, NO₂, CO₂H, CO₂C₁₋₆alkyl, CO₂NH₂, CO₂NH(C₁₋₆alkyl), CO₂N(C₁₋₆alkyl)₂, OH, alkoxy, acyl, acetyl, halomethyl, trifluoromethyl, benzyloxy, phenoxy, NH₂, NH(C₁₋₆alkyl) or N(C₁₋₆alkyl)₂.

As used herein, the term “hctcrocyclyl” refers to a cyclic, aliphatic or aromatic radical containing at least one heteroatom independently selected from O, N or S, Examples of suitable heterocyclyl groups include furyl, dioxolanyl, dioxanyl, dithianyl, dithiolanyl, pyridinyl, pyrimidinyl, pyrazolyl, piperidinyl, pyrrolyl, thyaphenyl, oxazolyl, imidazolyl, thiazolyl, isoxazolyl, isothiazolyl, quinolyl, isoquinolyl, indolyl, benzofuranyl, benzothiophenyl, triazolyl, tetrazolyl, oxadiazolyl and purinyl. Heterocyclyl groups may be optionally substituted one or more times by halo (eg, chloro, fluoro or bromo), CN, NO₂, CO₂H, CO₂C₁₋₆alkyl, CO₂NH₂, CO₂NH(C₁₋₆alkyl), CO₂N(C₁₋₆alkyl)₂, OH, alkoxy, acyl, acetyl, halomethyl, trifluoromethyl, benzyloxy, phenoxy, NH₂, NH(C₁₋₆alkyl) or N(C₁₋₆alkyl)₂.

The term “salt, or prodrug” includes any pharmaceutically acceptable salt, ester, solvate, hydrate or any oilier compound which, upon administration to the recipient is capable of providing (directly or indirectly) a compound of Formula (I) as described herein. The term “pro-drug” is used in its broadest sense and encompasses those derivatives that are converted in vivo to the compounds of the invention. Such derivatives would readily occur to those skilled in the art, and include, for example, compounds where a free hydroxy group is converted into an ester, such as an acetate, or where a free amino group is converted into an amide. Procedures for acylating hydroxy or amino groups of the compounds of the invention are well known in the art and may include treatment of the compound with an appropriate carboxylic acid, anhydride or acylchloride in the presence of a suitable catalyst or base.

Suitable pharmaceutically acceptable salts include, but are not limited to, salts of pharmaceutically acceptable inorganic acids such as hydrochloric, sulphuric, phosphoric, nitric, carbonic, boric, sulfamic, and hydrobromic acids, or salts of pharmaceutically acceptable organic acids such as acetic, propionic, butyric, tartaric, maleic, hydroxymaleic, fumaric, maleic, citric, lactic, muck, gluconic, benzoic, succinic, oxalic, phenylacetic, methanesulphonic, toluenesulphonic, benzenesulphonic, salicyclic sulphanilic, aspartic, glutamic, edetic, stearic, palmitic, oleic, lauric, pantothenic, tannic, ascorbic and valeric acids.

Base salts include, but are not limited to, those formed with pharmaceutically acceptable cations, such as sodium, potassium, lithium, calcium, magnesium, ammonium and alkylammonium.

Basic nitrogen-containing groups may be quarternised with such agents as lower alkyl halide, such as methyl, ethyl, propyl, and butyl chlorides, bromides and iodides; dialkyl sulfates like dimethyl and diethyl sulfate; and others.

It will also be recognised that some compounds of formula (I) may possess asymmetric centres and are therefore capable of existing in more than one stereoisomeric form. The invention thus also relates to compounds in substantially pure isomeric form at one or more asymmetric centres eg., greater than about 90% cc, such as about 95% or 97% ee or greater than 99% ce, as well as mixtures, including racemic mixtures, thereof. Such isomers may be prepared by asymmetric synthesis, for example using chiral intermediates, or by chiral resolution.

A further aspect of the invention provides for the use of a compound of Formula (I) or a pharmaceutically acceptable salt or prodrug thereof in die manufacture of a medicament for the treatment of a disease or condition as above.

In a further aspect of the invention, there is provided a pharmaceutical composition comprising a compound of formula (I) together with a pharmaceutically acceptable carrier, diluent or excipient.

The formulation of such compositions is well known to those skilled in the art. The composition may contain pharmaceutically acceptable additives such as carriers, diluents or excipients. These include, where appropriate, all conventional solvents, dispersion agents, fillers, solid earners, coating agents, antifungal and antibacterial agents, dermal penetration agents, surfactants, isotonic and absorption agents and the like. It will be understood that the compositions of the invention may also include other supplementary physiologically active agents.

The carrier must be pharmaceutically acceptable in the sense of being compatible with the other ingredients of the composition and not injurious to the subject. Compositions include those suitable for oral, rectal, inhalational, nasal, transdermal, topical (including buccal and sublingual), vaginal or parenteral (including subcutaneous, intramuscular, intraspinal, intravenous and intradermal) administration. The compositions may conveniently be presented in unit dosage form and may be prepared by any methods well known in the art of pharmacy. Such methods include the step of bringing into association the active ingredient with the carrier which constitutes one or more accessory ingredients. In general, the compositions are prepared by uniformly and intimately bringing into association the active ingredient with liquid carriers or finely divided solid carriers or both, and then if necessary shaping the product.

Depending on the disease or condition to be treated, it may or may not be desirable for a compound of Formula (I) to cross the blood/brain barrier. Thus the compositions for use in the present invention may be formulated to be water or lipid soluble.

Compositions of the present invention suitable for oral administration may be presented as discrete units such as capsules, sachets or tablets each containing a predetermined amount, of the active ingredient; as a powder or granules; as a solution or a suspension in an aqueous or non-aqueous liquid; or as an oil-in-water liquid emulsion or a water-in-oil liquid emulsion. The active ingredient may also be presented as a bolus, electuary or paste.

A tablet may be made by compression or moulding, optionally with one or more accessory ingredients. Compressed tablets may be prepared by compressing in a suitable machine the active ingredient in a free-flowing form such as a powder or granules, optionally mixed with a binder (eg inert diluent, preservative, disintegrant (eg. sodium starch glycolate, cross-linked polyvinyl pyrrolidone, cross-linked sodium carboxymethyl cellulose)) surface-active or dispersing agent. Moulded tablets may be made by moulding in a suitable machine a mixture of the powdered compound moistened with an inert liquid diluent. The tablets may optionally be coated or scored and may be formulated so as to provide slow or controlled release of the active ingredient therein using, for example, hydroxypropylmethyl cellulose in varying proportions to provide the desired release profile. Tablets may optionally be provided with an enteric coating, to provide release in parts of the gut other than the stomach.

Compositions suitable for topical administration in the mouth include lozenges comprising the active ingredient in a flavoured base, usually sucrose and acacia or tragacanth gum; pastilles comprising the active ingredient in an inert basis such as gelatin and glycerin, or sucrose and acacia gum; and mouthwashes comprising the active ingredient in a suitable liquid earner.

The compounds of Formula (I) may also be administered intranasally or via inhalation, for example by atomiser, aerosol or nebulizer means.

Compositions suitable for topical administration to the skin may comprise the compounds dissolved or suspended in any suitable carrier or base and may be in the form of lotions, gel, creams, pastes, ointments and the like. Suitable carriers include mineral oil, propylene glycol, polyoxyethylene, polyoxypropylene, emulsifying wax, sorbitan monostearate, polysorbate 60, cetyl esters wax, cetearyl alcohol, 2-octyldodecanol, benzyl alcohol and water. Transdermal devices, such as patches, may also be used to administer the compounds of the invention.

Compositions for rectal administration may be presented as a suppository with a suitable carrier base comprising, for example, cocoa butter, gelatin, glycerin or polyethylene glycol.

Compositions suitable for vaginal administration may be presented as pessaries, tampons, creams, gels, pastes, foams or spray formulations containing in addition to the active ingredient such carriers as are known in the art to be appropriate.

Compositions suitable for parenteral administration include aqueous and non-aqueous isotonic sterile injection solutions which may contain anti-oxidants, buffers, bactericides and solutes which render the composition isotonic with the blood of the intended recipient; and aqueous and non-aqueous sterile suspensions which may include suspending agents and thickening agents. The compositions may be presented in unit-dose or multi-dose sealed containers, for example, ampoules and vials, and may be stored in a freeze-dried (lyophilised) condition requiring only the addition of the sterile liquid carrier, for example water for injections, immediately prior to use. Extemporaneous injection solutions and suspensions may be prepared from sterile powders, granules and tablets of the kind previously described.

Preferred unit dosage compositions are those containing a daily dose or unit, daily sub-dose, as herein above described, or an appropriate fraction thereof, of the active ingredient.

It should be understood that in addition to the active ingredients particularly mentioned above, the compositions of this invention may include other agents conventional in the art having regard to the type of composition in question, for example, those suitable for oral administration may include such further agents as binders, sweeteners, thickeners, flavouring agents, disintegrating agents, coating agents, preservatives, lubricants and/or time delay agents. Suitable sweeteners include sucrose, lactose, glucose, aspartame or saccharine. Suitable disintegrating agents include corn starch, methylcellulose, polyvinylpyrrolidone, xanthan gum, bentonite, alginic acid or agar. Suitable flavouring agents include peppermint oil, oil of wintergreen, cherry, orange or raspberry flavouring. Suitable coating agents include polymers or copolymers of acrylic acid and/or methacrylic acid and/or their esters, waxes, fatty alcohols, zein, shellac or gluten. Suitable preservatives include sodium benzoate, vitamin E, alpha-tocopherol, ascorbic acid, methyl paraben, propyl paraben or sodium bisulphite. Suitable lubricants include magnesium stearate, stearic acid, sodium oleate, sodium chloride or talc. Suitable time delay agents include glyceryl mono stearate or glyceryl distearate.

In a further aspect, the present invention provides a method of inhibiting cytokine or biological activity of MIF comprising contacting MIF with a cytokine or biological activity inhibiting effective amount of a compound of formula (I), or a pharmaceutically acceptable salt or prodrug thereof.

In another aspect, the invention provides a method of treating, preventing or diagnosing a disease or condition wherein MIF cytokine or biological activity is implicated comprising the administration of a treatment, prevention or diagnostic effective amount of a compound of formula (I) or a pharmaceutically acceptable salt or prodrug thereof to a subject in need thereof.

In a further aspect, there is provided the use of a compound of formula (I) or a pharmaceutically acceptable salt or prodrug thereof in the manufacture of a medicament for the treatment, prevention or diagnosis of a disease or condition wherein MIF cytokine or biological activity is implicated.

As used herein, MIF includes human or other animal MIF and derivatives and naturally occurring variants thereof which at least partially retain MIF cytokine or biological activity. Thus, the subject to be treated may be human or other animal such as a mammal. Non-human subjects include, but are not limited to primates, livestock animals (eg sheep, cows, horses, pigs, goats), domestic animals (eg dogs, cats), birds and laboratory test animals (eg mice rats, guinea pigs, rabbits). MIF is also expressed in plants (thus “MIF” may also refer to plant MIF) and where appropriate, compounds of Formula (I) may be used in botanical/agricultural applications such as crop control.

Reference herein to “cytokine or biological activity” of MIF includes the cytokine or biological effect on cellular function via autocrine, endocrine, paracrine, cytokine, hormone or growth factor activity or via intracellular effects.

In another aspect, the invention provides a method of treating or preventing a disease or condition wherein MIF cytokine or biological activity is implicated comprising:
administering to a mammal a compound of formula (I) and a second therapeutic agent.

In a preferred embodiment of this aspect of the invention, the second therapeutic agent is a glucocorticoid compound.

In another aspect, the present invention provides a method of prophylaxis or treatment of a disease or condition for which treatment with a glucocorticoid is indicated, said method comprising: administering to a mammal a glucocorticoid and a compound of formula (I).

In yet another aspect, the present invention provides a method of treating steroid-resistant diseases comprising administering to a mammal a glucocorticoid and a compound of formula (I).

In a further aspect, the present invention provides a method of enhancing the effect of a glucocorticoid in mammals comprising administering a compound of formula (I) simultaneously, separately or sequentially with said glucocorticoid.

In yet a further aspect, the present invention provides a composition comprising a glucocorticoid and a compound of formula (I).

In a further aspect of the invention there is provided a use of a glucocorticoid in the manufacture of a medicament for administration with a compound of formula (I) for the treatment or prophylaxis of a disease or condition for which treatment with a glucocorticoid is indicated.

In yet a further aspect of the invention there is provided a use of a compound of formula (I) in the manufacture of a medicament for administration with a glucocorticoid for the treatment or prophylaxis of a disease or condition for which treatment of a glucocorticoid is indicated.

In yet a further aspect of the invention there is provided a use of a glucocorticoid and a compound of formula (I) in the manufacture of a medicament for the treatment or prophylaxis of a disease or condition for which treatment with a glucocorticoid is indicated.

Preferably the amount of glucocorticoid used in the methods, ruses and compositions of the invention is less than the amount which would be effective in the absence of the compound of formula (I). In the treatment of steroid-resistant diseases or conditions which are not responsive, to glucocorticoids, any amount of glucocorticoid which is effective in combination with a compound of formula (I) is considered less man the amount which would be effective in the absence of a compound formula (I). Accordingly, the invention provides a steroid-sparing therapy.

The term “disease or condition for which treatment with a glucocorticoid is indicated” refers to diseases or conditions which are capable of being treated by administration of a glucocorticoid including but not limited to autoimmune diseases, tumours, or chronic or acute inflammatory diseases. Examples of such diseases or conditions include:
rheumatic diseases (including but not limited to rheumatoid arthritis, osteoarthritis, psoriatic arthritis) spondyloarthropathies (including but not limited to ankylosing spondylitis, reactive arthritis, Reiter's syndrome), crystal arthropathies (including but not limited to gout, pseudogout, calcium pyrophosphate deposition disease), Lyme disease, polymyalgia rheumatica;
connective tissue diseases (Including but not limited to systemic lupus erythematosus, systemic sclerosis, polymyositis, dermatomyositis, Sjögren's syndrome);
vasculitides (including but not limited to polyarteritis nodosa, Wegener's granulomatosis, Churg-Strauss syndrome);
inflammatory conditions including consequences of trauma or ischaemia;
sarcoidosis;
vascular diseases including atherosclerotic vascular disease and infarction, atherosclerosis, and vascular occlusive disease (including but not limited to atherosclerosis, ischaemic heart disease, myocardial infarction, stoke, peripheral vascular disease), and vascular stent restenosis;
autoimmune diseases (including but not limited to diabetes mellitus, thyroiditis, myasthenia gravis, sclerosing cholangitis, primary biliary cirrhosis);
pulmonary diseases (including but not limited to diffuse interstitial lung diseases, pneumoconioses, fibrosing alveolitis, asthma, bronchitis, bronchiectasis, chronic obstructive pulmonary disease, adult respiratory distress syndrome);
cancers whether primary or metastatic (including but not limited to prostate cancer, colon cancer, lymphoma, lung cancer, melanoma, multiple myeloma, breast cancer, stomach cancer, leukaemia, cervical cancer and metastatic cancer);
renal diseases including glomerulonephritis, interstitial nephritis;
disorders of the hypothalamic-pituitary-adrenal axis;
nervous system disorders including multiple sclerosis, Alzheimer's disease;
diseases characterised by modified angiogenesis (eg diabetic retinopathy, rheumatoid arthritis, cancer), endometrial function (menstruation, implantation, endometriosis);
complications of infective disorders including endotoxic (septic) shock, exotoxic (septic) shock, infective (true septic) shock, malarial complications, other complications of infection, pelvic inflammatory disease;
transplant rejection, graft-versus-host disease;
allergic diseases including allergies, atopic diseases, allergic rhinitis;
bone diseases (eg osteoporosis, Paget's disease);
skin diseases including psoriasis, atopic dermatitis, UV(B)-induced dermal cell activation (eg sunburn, skin cancer); pain, testicular dysfunctions and wound healing;
gastrointestinal diseases including inflammatory bowel disease (including but not limited to ulcerative colitis, Crohn's disease), peptic ulceration, gastritis, oesophagitis, liver disease (including but not limited to cirrhosis, hepatitis).

These diseases or conditions may also include steroid-resistant diseases or conditions where treatment with a glucocorticoid is indicated, but where the glucocorticoid is ineffective or is not as effective as expected.

The methods of the invention are preferably performed in a steroid-sparing manner. The term “steroid-sparing” refers to a combination therapy method that allows a reduction in the amount of glucocorticoid administered while still providing an effective therapy for the disease or condition being treated or prevented.

Steroid-resistant diseases or conditions are diseases or conditions for which treatment with a glucocorticoid is indicated, but where the glucocorticoid is ineffective or is not as effective as expected. This term encompasses diseases or conditions for which the effective dose of glucocorticoid results in unacceptable side effects and/or toxicity. Some steroid-resistant diseases or conditions may require a dosage of glucocorticoid so large that they are considered non-responsive and therefore are not able to be successfully treated with glucocorticoids. Some steroid-resistant diseases or conditions may require a large dosage of glucocorticoid to achieve only a small effect on the symptoms of the disease or condition. Furthermore, some patients, diseases or conditions present with symptoms that do not respond to treatment with a glucocorticoid, or may become less sensitive to glucocorticoid treatment over time.

Glucocorticoids are a group of steroid hormones, which are used to treat or prevent a wide range of diseases or conditions. Suitable glucocorticoids may be synthetic or naturally occurring and include but are not limited to prednisolone, prednisone, cortisone acetate, beclamethasone, fluticasone, hydrocortisone, dexamethasone, methyl prednisolone, triamcinolone, budesonide and betamethasone.

In preferred embodiments of the invention, the glucocorticoid used is selected from prednisone, prednisolone, hydrocortisone, fluticasone, beclamethasone, betamethasone, methyl prednisolone, budesonide, triamcinolone, dexamethasone and cortisone. Most preferably, the glucocorticoid is selected from prednisone, prednisolone, methyl prednisolone, fluticasone and beclamethasone. Beclamethasone and fluticasone are particularly preferred for treating asthma. Prednisone, prednisolone and methyl prednisolone are particularly preferred in the treatment of systemic or local inflammatory diseases.

The amounts of glucocorticoid and compound of formula (I) are selected such that in combination they provide complete or partial treatment or prophylaxis of a disease or condition for which a glucocorticoid is indicated. The amount of compound formula (I) is preferably an amount that will at least partially inhibit the cytokine or biological activity of MIF. The amount of glucocorticoid is preferably less than the amount required in the absence of the compound of formula (I). The amounts of glucocorticoid and compound of formula (I) used in a treatment or therapy are selected such that in combination they at least partially attain the desired therapeutic effect, or delay onset of, or inhibit the progression of, or halt or partially or fully reverse the onset or progression of the disease or condition being treated. The amounts of glucocorticoid and compound of formula (I) used in the prophylaxis of a disease or condition are selected such that in combination they at least partially prevent or delay the onset of the disease or condition. Dosing may occur at intervals of minutes, hours, days, weeks, months or years or continuously over any one of these periods.

Suitable doses of a compound of formula (I) may lie within the range of about 0.1 ng per kg of body weight to 1 g per kg of body weight per dosage. The dosage is preferably in the range of 1 μg to 1 g per kg of body weight per dosage, such as is in the range of 1 mg to 1 g per kg of body weight per dosage. In one embodiment, the dosage is in the range of 1 mg to 500 mg per kg of body weight per dosage. In another embodiment, the dosage is in the range of 1 mg to 250 mg per kg of body weight per dosage. In yet another preferred embodiment, the dosage is in the range of 1 mg to 100 mg per kg of body weight per dosage, such as up to 50 mg per kg of body weight per dosage. In yet another embodiment, the dosage is in the range of 1 μg to 1 mg per kg of body weight per dosage.

Suitable dosage amounts of glucocorticoids will depend, in part, on the mode of administration and whether the dosage is being administered in a single, daily or divided dose, or as a continuous infusion. When administered orally, intravenously, intramuscularly, intralesionally or intracavity (eg. intra-articular, intrathecal, intrathoracic), dosages are typically between 1 mg to 1000 mg, preferably 1 mg to 100 mg, more preferably 1 mg to 50 mg or 1 mg to 10 mg per dose. When administered topically or by inhalation as a single, daily or divided dose, dosages are typically 1 ng to 1 μg, 1 ng to 1 mg or 1 pg to 1 μg.

Suitable dosage amounts and dosing regimens can be determined by the attending physician or veterinarian and may depend on the desired level of inhibiting activity, the particular condition being treated, the severity of the condition as well as the general age, health and weight of the subject.

The glucocorticoid and compound of formula (I) may be administered simultaneously or sequentially. The active ingredients may be administered alone but are preferably administered as a pharmaceutically acceptable composition or separate pharmaceutically acceptable compositions.

The formulation of such compositions is well known to those skilled in the art and are described above in relation to compounds of formula (I). The composition or compositions may contain pharmaceutically acceptable additives such as carriers, diluents or excipients. These include, where appropriate, all conventional solvents, dispersion agents, fillers, solid carriers, coating agents, antifungal and antibacterial agents, dermal penetration agents, surfactants, isotonic and absorption agents and the like. It will be understood that the compositions of the invention may also include other supplementary physiologically active agents.

Preferred unit dosage compositions are those containing a daily dose or unit, daily sub-dose, as herein above described, or an appropriate fraction thereof, of the glucocorticoids and/or

The compounds of formula (I), either as the only active agent or together with another active agent, eg; a glucocorticoid, may also be presented for use in veterinary compositions. These may be prepared by any suitable means known in the art. Examples of such compositions include those adapted for;
oral administration, external application (eg drenches including aqueous and non-aqueous solutions or suspensions), tablets, boluses, powders, granules, pellets for admixture with feedstuffs, pastes for application to the tongue;
parenteral administration, eg subcutaneous, intramuscular or intravenous injection as a sterile solution or suspension; and
topical application eg creams, ointments, gels, lotions, etc.

By virtue of their ability to bind to or antagonize MIF, compounds of Formula (I) or salts or derivatives thereof may be used as laboratory or diagnostic or in vivo imaging reagents. Typically, for such use the compounds would be labelled in some way, for example, radio isotope, fluorescence or colorimetric labelling, or be chelator conjugated. In particular, compounds of Formula (I) could be used as part of an assay system for MIF or as controls in screens for identifying other inhibitors. Those skilled in the art are familiar with such screens and could readily establish such screens using compounds of Formula (I). Those skilled in the art will also be familiar with the use of chelate conjugated molecules for in vivo diagnostic imaging.

inhibitors of MIF may also be used in implantable devices such as stents. Accordingly, in a further aspect the present invention provides an implantable device, preferably a stent, comprising:
(i) a reservoir containing at least one compound of formula (I); and
(ii) means to release or elute the inhibitor from the reservoir

There is further provided a method for inhibiting the cytokine or biological activity of MIF in a subject comprising the step of implanting an implantable device according to the invention in the subject.

Preferably, the method is for inhibiting the cytokine or biological activity of MIF in a local region of the subject and the device is implanted within or proximate to the local region of the subject.

In a yet further aspect, the present invention provides a method of treating, preventing or diagnosing a disease or condition wherein MIF cytokine activity is implicated comprising the step of implanting an implantable device according to the invention in a subject in need thereof.

Preferably, the disease or condition is confined to a local region or the subject and the device is implanted with in or proximate to the local region.

The present invention further provides an angioplasty stent, for inhibiting the onset of restenosis, which comprises an angioplastic stent operably coated with a prophylactically effective dose of a composition comprising at least one compound of formula (I).

Angioplastic stents, also known by other terms such as “intravascular stents” or simply “stents”, are well known in the art. They are routinely used to prevent vascular closure due to physical anomalies such as unwanted inward growth of vascular tissue due to surgical trauma. They often have a tubular, expanding lattice-type structure appropriate for their function, and can optionally be biodegradable.

In this invention, the stent can be operably coated with at least one compound or formula (I) using any suitable means known in the art. Here, “operably coating” a stent means coating it in a way that permits the timely release of the compound(s) of formula (I) into the surrounding tissue to be treated once the coated stent is administered. Such coating methods, for example, can use the polymer polypyrrole.

The present invention further provides a method for inhibiting the onset of restenosis in a subject undergoing angioplasty, which comprises topically administering a stent according to the present invention to the subject at around the time of the angioplasty.

As used herein, administration “at around the time of angioplasty” can be performed during the procedure, or immediately before or after the procedure. The administering can be performed according to known methods such as catheter delivery.

There is further provided a method of reducing the severity of stent restenosis in the vicinity of a stent comprising the use of a stent according to the present invention.

The construction of stents that, release or elute a pharmaceutical active is known to those skilled in the art. The standard approach is to use current highly refined metallic stent designs with polymer materials that release the active in a controlled manner. Several polymer materials have been used for the coating of stents to permit the elution of drags. These include bioerodible polymers such as poly-L lactic acid, biostable polymers such as polyurethane derivatives and silicone-based polymers, as well as hydrogels. It will be recognised by those skilled in the art that the function of a drug-eluting stent requires the drug to be bound to the stent or its polymer or other coating in such a way as to allow steady release of drug over a period of time, and that the drug is able to be locally absorbed into cells in the vessel and stent lumen. The optimum stent coating material and delivery parameters vary according to the tissue retention of the drug, such that rapid release of a tissue-retained drug can have long lasting effects, whereas a drug retained in tissues for a shorter time would need to be released over a longer period. A person skilled in the art would be able to select appropriate materials and conformations of stent for a particular purpose and particular small molecule inhibitor.

### Proposed Methods of Synthesis

Commercially available starting materials for the preparation of examples include the unsubstituted heterocycles where X and Y are a combination of CH₂, O, NH and S (sec Scheme 1). In cases where Z is C═O these include; benzimidazol-2-one (2-(2-benzoxazolinone) and benzothiazol-2-one (2-hydroxybenzothiazole). In cases where Z is C═NH these include the tautomeric compounds; 2-aminobenzimidazole, 2-aminobenzothiazole and 2-aminobenzoxazole. Further elaboration of the heterocyclic ring may be made by alkylation of basic functionalities using reagents such as methyl iodide or dimethyl sulfate in the presence of base.

Friedel-Crafts acylation of these heterocycles with haloalkyl acid halides and aluminium chloride in solvents such as 1,2-dichloroethane or N,N-dimethylformamide would afford a range of haloalkyl ketones as shown in Scheme 1. The haloalkyl acid halides for t=1-5 are available commercially, while longer homologues may be prepared by treatment of the more widely available hydroxy-acids with a combination of HBr and oxalyl chloride, or by treatment with thionyl chloride.

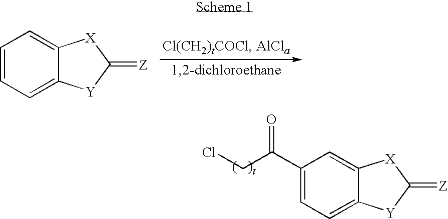

Displacement of the halogen with an appropriately functionalized sulfur or nitrogen nucleophile in the presence of a non-nucleophilic base would give rise to a range of examples where Q is NH or S. In cases where a nitrogen nucleophile is used this could be either a primary or secondary amine, affording secondary or tertiary amine examples respectively. In eases where a sulfur nucleophile is used oxidation of the resulting sulfide with reagents such as hydrogen peroxide would generate sulfoxide examples (u=1). Further oxidation using a stronger oxidant such as potassium permanganate or an additional equivalent of hydrogen peroxide would give rise to sulfone examples (u=2) (see Scheme 2).

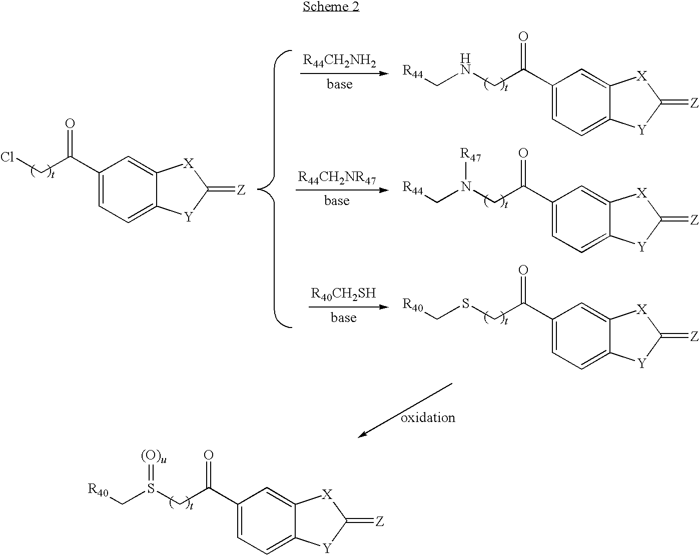

Displacement of the halogen by oxygen nucleophiles can be achieved by suitable protection, if necessary, of any additional functionality on the alcohol, followed by treatment with sodium hydride or sodium metal to generate the more nucleophilic alkoxide anion. This procedure would allow access to the range of examples that have Q=O (sec Scheme 3).

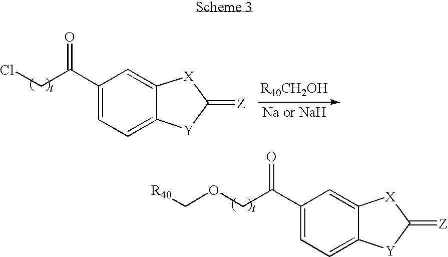

If in place of the haloalkyl acid halides shown in Scheme 1, cyclic anhydrides or alkoxycarbonyl acid halides are used, then a series of keto-acids may be prepared (Scheme 4). Selective reduction of the ketone functionality can be achieved with a selection of reagents including; zinc amalgam, triethylsilane and sodium borohydride, to afford the corresponding carboxylic acids.

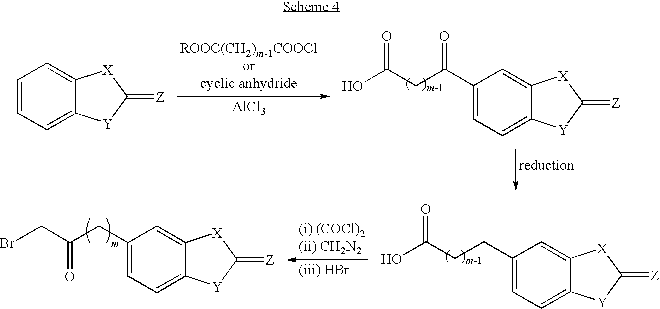

Conversion of the acid to the acid chloride followed by treatment with diazomethane and then HBr, affords bromoalkyl ketones (t=1) which can, be used to prepare examples as previously illustrated in Schemes 2 (Q=N, S) and 3 (Q=O),

As described above, compounds of Formula (I) may be prepared using the methods depicted or described herein or known in the art. It will be understood that minor modifications to methods described herein or blown in the art may be required to synthesize particular compounds of Formula (I). General synthetic procedures applicable to the synthesis of compounds may be found in standard references such as Comprehensive Organic Transformations, R. C. Larock, 1989, VCH Publishers and Advanced Organic Chemistry, J. March, 4th Edition (1992), Wiley InterScience, and references therein. It will also be recognised that certain reactive groups may require protection and deprotection during the synthetic process. Suitable protecting and deprotecting methods for reactive functional groups are known in the art for example in Protective Groups in Organic Synthesis, T. W. Green & P. Wutz, John Wiley & Son, 3rd Edition, 1999.

In order that the nature of the present invention may be more clearly understood, preferred forms thereof will now be described with reference to the following non-limiting examples.

### SYNTHETIC EXAMPLES

### General Experimental

Melting points are uncorrected. Proton nuclear magnetic resonance (¹H nmr) spectra were acquired on either a Bruker Avance 300 spectrometer at 300 MHz, or on a Varian Inova spectrometer at 400 MHz, using the dueterated solvents indicated. Low resolution mass spectrometry analyses were performed using a Micromass Platform II single quadrapole mass spectrometer equipped, with an electrospray (ESI) or atmospheric pressure chemical ionization (APCI) ion source. Sample management was facilitated by an Agilent 1100 series HPLC system.

Commercially sourced starting materials and solvents were used without further purification.

The following abbreviations have been used: mp, melting point; DCE, 1,2-dichloroethane; DMF, N,N-dimethylformamide; THF, tetrahydrofuran; TLC, thin layer chromatography; SiO₂, silica gel; dmso, dimethylsulfoxide; DCM, dichloromethane; MeOH, methanol.

### Example 1

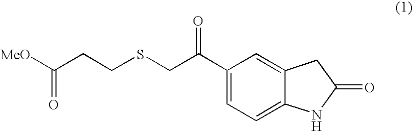

### Preparation of methyl 3-((2-oxo-2-(2-oxo-2,3-dihydro-1H-indol-5-yl)ethyl)thio)propanoate (1)

### (i) 5-Bromoacetyloxindole (U.S. Pat. No. 5,849,710)

To a suspension or anhydrous aluminium chloride (11.4 g, 85 mmol) in 1,2-dichloroethane (25 ml) stirred at 0 PC was added bromoacetyl bromide (5.9 ml, 68 mmol) dropwise. After 1 h a suspension of oxindole (4.52 g, 34 mmol) in 1,2-dichloroethane (25 ml) was added and stirring continued for 2 h at 0° C. then for 3 h at 50° C. The reaction mixture was cooled, poured onto ice/water (500 ml) and filtered to give 5-bromoacetyloxindole as a light brown solid (7.1 g, 82%) that was used without further purification.

### (ii) Methyl 3-((2-oxo-2-(2-oxo-2,3-dihydro-1H-indol-5-yl)ethyl)thio)propanoate (1)

To a suspension of 5-bromoacetyloxindole (4.15 g, 16.3 mmol) in N,N-dimethylformamide (20 ml) was added methyl 3-mercaptopropionate (2.14 ml, 19.6 mmol) and di-isopropylethylamine (6.1 ml, 35 mmol) resulting in a dark brown solution. The solution was stirred for 36 h at room temperature under an atmosphere of nitrogen then concentrated to give a yellow gum. Column chromatography (SiO₂) eluting with 20:1 chloroform/MeOH afforded a dark yellow compound that was recrystallised from methanol to give the ester (1) as a light yellow solid (3.3 g, 72%), mp. 106-108° C. (TLC: R_{F}=0.64 on SiO₂ with 9:1 chloroform/MeOH).

¹H nmr (300 MHz, d₆-dmso) δ 2.61, t (6.4 Hz), CH₂; 2.70, t (5.8 Hz), CH₂; 3.54, s, H3; 3.57, s, OMe; 3.95, s, SCH₂CO; 6.89, d (8.1 Hz), H7; 7.81, s, H4; 7.87, br d (8.4 Hz), H6; 10.74, br s, NH.

ESI (+ve) m/z 316 (M+Na, 20%), 294 (M+H, 100%).

### Example 2

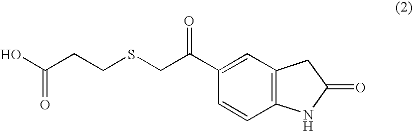

### Preparation of 3-((2-oxo-2-(2-oxo-2,3-dihydro)-1H-indol-5-yl)ethyl)thio)propanoic acid (2)

A solution of the methyl ester (1) (3.0 g, 10.2 mmol) in concentrated hydrochloric acid (30 ml) was heated to reflux for 5 min then cooled to room temperature to give a yellow precipitate. The solid was filtered, washed with water and recrystallised from methanol to give the acid (2) as a light yellow solid (1.50 g, 52%), mp. 182-184° C. (TLC: R_{F}=0.31 on SiO₂ with 9:1 chloroform/MeOH).

¹H nmr (300 MHz, d₆-dmso) δ 2.5, obscured, CH₂; 2.65, t (7.1 Hz), CH₂; 3.54, s, H3; 3.94, s, SCH₂CO; 6.89, d (8.1 Hz), 117; 7.82, s, H4; 7.87, d (8.4 Hz), H6; 10.74, br s, NH; 12.21, br s, COOH.

ESI (+ve) m/z 280 (M+H, 100%). ESI (−ve) m/z 278 (M−H, 100%).

### Example 3

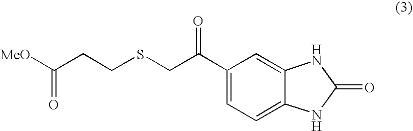

### Preparation of methyl 3-((2-oxo-2-(2-oxo-2,3-dihydro-1H-benzimidazol-5-yl)ethyl)thio)propanoate (3)

### (i) 5-(Chloroacetyl)-1,3-dihydro-2H-benzimidazol-2-one (WO 92/50070)

Anhydrous aluminium chloride (7.5 g, 60 mmol) was crushed to a powder under nitrogen then suspended in 1,2-dichloroethane (10 ml). The suspension was cooled to 0° C. and chloroacetyl chloride (3.6 ml, 45 mmol) added dropwise. After stirring at 0° C. for 30 min 2-hydroxybenzimidazole (3.0 g, 22.4 mmol) was added portion-wise with additional 1,2-dichloroethane (5 ml). The reaction mixture was heated for 2 h at 50-55° C. under nitrogen with vigorous stirring during which time the green-blue suspension became a dark solution. After stirring for 16 h at room temperature the mixture was poured onto ice (100 g) and the resulting grey precipitate filtered. The solid was washed with water then ethyl acetate and dried under vacuum to give 5-(chloroacetyl)-1,3-dihydro-2H-benzimidazol-2-one as a light grey powder (4.7 g, 100%) that was used without further purification.

### (ii) Methyl 3-((2-oxo-2-(2-oxo-2,3-dihydro-1H-benzimidazol-5-yl)ethyl)thio))propanoate (3)

To a solution of methyl 3-mercaptopropionate (0.57 g, 4.7 mmol) in dry tetrahydrofuran (15 ml) was added 5-(chloroacetyl)-1,3-dihydro-2H-benzimidazol-2-one (1.0 g, 4.7 mmol) followed by anhydrous potassium carbonate (3.3 g, 23.9 mmol, 5 eq) and the mixture stirred at room temperature for 24 h. The reaction mixture was partitioned between ethyl acetate (50 ml) and water (50 ml) and the aqueous layer re-extracted with fresh ethyl acetate (50 ml). The combined organic extract was then washed with water (2×50 ml), brine (1×50 ml), dried (MgSO₄) and the solution concentrated by rotary evaporator. Reducing the volume to 20-30 ml resulted in the formation of a precipitate which after chilling in ice was filtered to give the ester (3) as a red-brown solid (0.959 g, 69%), mp. 185-187° C. (TLC: R_{F}=0.47 on SiO₂ with 17:3 DCM/McOH).

¹H nmr (300 MHz, d₆-dmso) δ 2.62, m, CH₂; 2.72, m, CH₂; 3.58, s, OMe; 3.99, s, SCH₂CO; 7.00, d (8.1 Hz), H7; 7.48, d (1.5 Hz), H4; 7.67, dd (1.5, 8.1 Hz), H6; 10.86, s, NH; 11.03, s, NH.

ESI (+ve) m/z 317 (M+Na, 15%), 295 (M+H, 100%). ESI (−ve) m/z 293 (M−H, 100%).

### Example 4

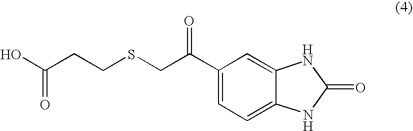

### Preparation of 3-((2-oxo-2-(2-oxo-2,3-dihydro-1H-benzimidazol-5-yl)ethyl)thio)propanoic acid (4)

To a solution of the methyl ester (3) (300 mg, 1.02 mmol) in methanol (75 ml) was added 1M sodium hydroxide solution (25 ml) and die solution stirred at room temperature for 4 h. The bulk of the methanol was then removed by rotary evaporator and the aqueous solution acidified with 1M hydrochloric acid solution (25 ml). The cloudy suspension was then extracted with ethyl acetate (3×50 ml), die extract washed with brine (1×100 ml), dried (MgSO₄) and evaporated to give the acid (4) as a pale yellow powder (0.229 g, 80%), mp. 212-215° C. (TLC: R_{F}=0.09 on SiO₂ with 17:3 DCM/McOH). ¹H nmr (300 MHz, d₆-dmso) δ 2.53, t (6.9 Hz), CH₂; 2.68, t (6.9 Hz), CH₂; 3.98, s, SCH₂CO; 7.00, d (8.1 Hz), H7; 7.48, d (1.2 Hz), H4; 7.67, dd (1.8, 8.1 Hz), H6; 10.86, s, NH; 11.02, s, NH; 12.21, br s, COOH.

ESI (+ve) m/z 303 (M+Na, 70%), 281 (M+H, 100%). ESI (−ve) m/z 279 (M−H, 100%).

### Example 5

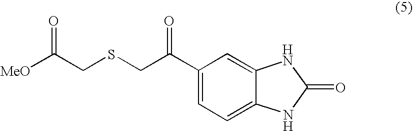

### Preparation of methyl ((2-oxo-2-(2-oxo-2,3-dihydro-1H-benzimidazol-5-yl)ethyl)thio)acetate (5)

To a solution of methyl thioglycolate (0.426 g, 4.01 mmol) in dry tetrahydrofuran (30 ml) was added 5-(chloroacetyl)-1,3-dihydro-2H-benzimidazol-2-one (0.80 g, 3.8 mmol) from example 3, followed by anhydrous potassium carbonate (2.62 g, 5 eq). The mixture was stirred at room temperature for 90 h then partitioned between ethyl acetate (100 ml) and water (100 ml) and the aqueous layer extracted further with ethyl acetate (1×100 ml). The combined organic extract was washed with water (1×100 ml), brine (1×100 ml), dried (MgSO₄) and the volume reduced by rotary evaporator to 30-40 ml. Reduction of the volume afforded a solid that was filtered off and dried under vacuum to give the ester (5) as a light orange powder (0.781 g, 73%), mp. 177-178.5° C. (TLC: R_{F}=0.50 on silica gel with 17:3 DCM/MeOH).

¹H nmr (300 MHz, d₆-dmso) δ 3.40, s, OOCCH₂S; 3.61, s, OMe; 4.11, s, SCH₂CO; 7.01, d (8.1 Hz), H7; 7.47, app s, H4; 7.66, dd (1.3, 8.1), H6; 10.87, s, NH; 11.04, s, NH.

ESI (+ve) m/z 303 (M+Na, 27%), 281 (M+H, 100%). ESI (−ve) m/z 219 (M−H, 100%).

### Example 6

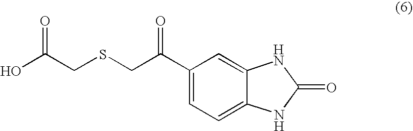

### Preparation of ((2-oxo-2-(2-oxo-2,3-dihydro-1H-benzimidazol-5-yl)ethyl)thio)acetic acid (6)

To a solution of the methyl ester (5) (0.30 g, 1.07 mmol) in methanol (75 ml) was added 1M sodium hydroxide solution (25 ml) and the mixture stirred at room temperature for 3.5 h. The bulk, of the methanol was then removed and the remaining aqueous solution acidified with 1M hydrochloric acid (25 ml) while stirring at 0° C. n-Butanol (50 ml) and brine (50 ml) were then added and the aqueous layer re-extracted with n-butanol (50 ml). The combined organic extract was washed with water (1×100 ml), brine (1×100 ml), dried (MgSO₄) and evaporated to give the acid (6) as an olive-green powder (0.238 g, 84%) which was recrystallised from methanol, mp. 230° C. (dec).

¹H nmr (300 MHz, d₆-dmso) δ 3.24, s, OOCCH₂S; 4.06, s, SCH₂CO; 7.00 d (8.1 Hz), H7; 7.48, d (1.5 Hz), 7.67, dd (1.5, 8.1 Hz), H6; 10.89, s, NH; 11.06, s, NH.

ESI (+ve) m/z 311 (M+2Na—H, 20%), 289 (M+Na, 45%), 267 (M+H, 55%). ESI (−ve) m/z 287 (M+Na-2H, 20%), 265 (M−H, 100%).

### Example 7

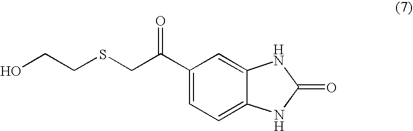

### Preparation of 5-(((2-hydroxyethyl)thio)acetyl)-1,3-dihydro-2H-benzimidazol-2-one (7)

To a solution of 2-mercaptoethanol (0.30 g, 3.8 mmol) in dry tetrahydrofuran (30 ml) was added 5-(chloroacetyl)-1,3-dihydro-2/Y-benzimidazol-2-one (0.80 g, 3.8 mmol) from example 3, followed by anhydrous potassium carbonate (2.62 g, 5 eq). The mixture was stirred at room temperature for 18 h then the reaction mixture partitioned between ethyl acetate (100 ml) and water (100 ml) and the aqueous layer further extracted with ethyl acetate (1×100 ml). The combined organic extract was washed with water (1×100 ml), brine (1×100 ml), dried (MgSO₄) and the volume reduced to 30-40 ml. Reduction of the volume afforded a precipitate that was filtered off and dried under vacuum to give the alcohol (7) as a light olive-green powder (0.285 g, 30%), mp. 320° C. (dec) (TLC: R_{F}=0.31 on SiO₂ with 17:3 DCM/MeOH).

¹H nmr (300 MHz, d₆-dmso) δ 2.58, t (6.8 Hz), CH₂S; 3.51, dt (5.7, 6.6 Hz), HOCH₂; 3.95, s, SCH₂CO; 4.74, t (5.4 Hz), HO; 7.00, d (8.1 Hz), 117; 7.48, d (1.2 Hz), H4; 7.66, dd (1.6, 8.2 Hz), H6; 10.86, br s, NH; 11.02, br s, NH.

ESI (+ve) m/z 275 (M+Na, 45%), 253 (M+H, 100%). ESI (−ve) m/z 251 (M−H, 100%).

### Example 8

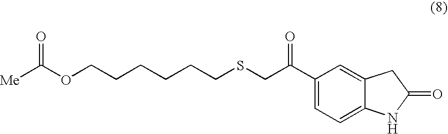

### Preparation of 6-((2-oxo-2-(2-oxo-2,3-dihydro-1H-indol-5-yl)ethyl)thio)hexyl acetate (8)

A suspension of sodium hydride (0.237 g, 60% dispersion, 5.92 mmol) in anhydrous N,N-dimethylformamide (7 ml) was stirred at 0° C. for 5 min under nitrogen. 6-Mercapto-1-hexanol (0.059 ml, 0.43 mmol) was added and storing continued at 0° C. for 20 min then 5-chloroacetyloxindole (0.099 g, 0.474 mmol) was added and stirring continued at 0°G for a further 1 h. The suspension was then partitioned between ethyl acetate and water and the aqueous phase acidified with 1M hydrochloric acid and extracted with ethyl acetate. The combined organic phases were washed with 1M hydrochloric acid, water, brine, dried (MgSO₄) and concentrated to give a sticky yellow solid (0.231 g). Purification by column chromatography (SiO₂) eluting with 99:1 DCM/MeOH afforded the ester (8) as a white solid (0.085 g, 51%), mp. 86-87° C. (TLC: R_{F}=0.44 on SiO₂ with 9:1 DCM/McOH).

¹H nmr (300 MHz, CDCl₃) δ 1.38, m, 2×CH₂; 1.57, m, 2×CH₂; 2.04, s, Me; 2.57, t (7.2 Hz), CH₂S; 3.60, s, H3; 3.73, s, SCH₂CO; 4.04, t (6.9 Hz), OCH₂; 6.92, d (8.1 Hz), H7; 7.69, br s, NH; 7.89, br s, H4; 7.93, br d (8.4 Hz), H6.

ESI (+ve) m/z 372 (M+Na, 20%), 350 (M+H, 100%). ESI (−ve) m/z 348 (M−H, 10%).

### Example 9

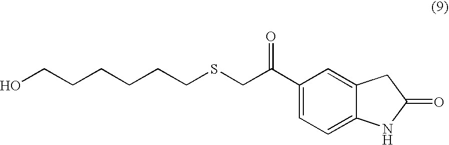

### Preparation of 5-(((6-hydroxyhexyl)thio)acetyl)-1,3-dihydro-2H-indol-2-one (9)

After elution with 99:1 DCM/MeOH as described in example 8, further elution with 95:5 DCM/MeOH afforded the alcohol (9) as a pale beige solid (0.048 g, 30%), mp. 105-108° C. (TLC: R_{F}=0.35 on SiO₂ with 9:1 DCM/MeOH).

¹H nmr (300 MHz, d₆-dmso) δ 1.30-1.55, m, 4×CH₂; 2.4, obscured, CH₂S; 3.35, dt (5.1, 6.4 Hz). OCH₂; 3.54, s, H3; 3.87, s, SCH₂CO; 4.28, t (5.2 Hz), OH; 6.89, d (8.1 Hz), H7; 7.81, br s, H4; 7.87, dd (1.5, 8.3 Hz), H6; 10.73, s, NH.

ESI <+ve) m/z 330 (M+Na, 25%), 308 (M+H, 70%). ESI (−ve) m/z 306 (M−H, 60%).

### Example 10

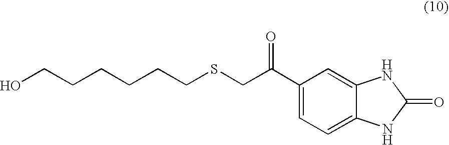

### Preparation of 5-(((6-hydroxyhexyl)thio)acetyl)-1,3-dihydro-2H-benzimidazol-2-one (10)

To a solution of 6-mercapto-1-hexanol (0.51 g, 3.8 mmol) in tetrahydrofuran (30 ml) was added 5-(chloroacetyl)-1,3-dihydro-2H-benzimidazol-2-one (0.80 g, 3.8 mmol) from example 3, followed by dry potassium carbonate (2.62 g, 19.0 mmol, 5 eq) and the suspension stirred at room temperature for 96 h. The reaction mixture was partitioned between water (100 ml) and ethyl acetate (80 ml) and the aqueous layer re-extracted with ethyl acetate (80 ml). The combined organic extract was washed with water (1×100 ml), brine (1×100 ml), dried (MgSO₄) and concentrated to a volume of 30-40 ml resulting in precipitation. The precipitate was filtered off to give the alcohol (10) as a pale yellow powder (0.658 g, 56%), mp. 180-181° C.

¹H nmr (300 MHz, d₆-dmso) δ 1.2-1.55, m, 4×CH₂; 2.5, obscured, CH₂S; 3.35, t (6.5 Hz), OCH₂; 3.89, s, SCH₂CO; 4.2, br s, OH; 6.99, d (8.1 Hz), H7; 7.48, d (1.2 Hz), H4; 7.66, dd (1.6, 8.2 Hz), H6; 10.85, s, NH; 11.02, s, NH.

ESI (+ve) m/z 331 (M+Na, 40%), 309 (M+H, 100%). ESI (−ve) m/z 307 (M−H, 100%).

### Example 11

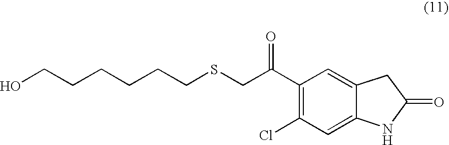

### Preparation of 6-chloro-5-(((6-hydroxyhexyl)thio)acetyl)-1,3-dihydro-2H-indol-2-one (11)

A suspension of 5-chloroacetyl-6-chlorooxindole (0.099 g, 0.407 mmol), 6-mercapto-1-hexanol (0,062 ml, 0.453 mmol) and potassium carbonate (0.059 g, 0.43 mmol) in acetonitrile was heated at reflux under nitrogen for 2.5 h then cooled to room temperature. The suspension was filtered and the filtrate concentrated to give a dark red-brown solid (0.163 g). The solid was nitrified by column chromatography (SiO₂) eluting with 100% DCM, 99:1 and 95:5 DCM/MeOH to give the alcohol (11) as a pale yellow solid (0.091 g, 65%), rap. 106-108° C. (TLC: R_{F}=0.42 on SiO₂ with 9:1 DCM/MeOH).

¹H nmr (300 MHz, CDCl₃) δ 1.38, m, 2×CH₂; 1.53, m, 2×CH₂; 2.54, br s, CH₂S; 3.56, s, H3; 3.64, t (6.3 Hz), OCH₂; 3.84, br s, SCH₂CO; 6.95, s, 117; 7.52, s, H4; 8.63, s, NH.

ESI (+ve) m/z 364/366 (M+Na, 25/8%), 342/344 (M+H, 100/30%). ESI (−ve) m/z 340/342 (M−H, 100/35%).

### Example 12

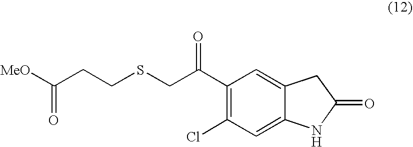

### Preparation of methyl 3-((2-(6-chloro-2-oxo-2,3-dihydro-1H-indol-5-yl)-2-oxoethyl)thio)propanoate (12)

A suspension of 5-chloroacetyl-6-chlorooxindole (0.100 g, 0.413 mmol), methyl 3-mercaptopropionate (0.050 ml, 0.45 mmol) and potassium carbonate (0.057 g, 0.41 mmol) in acetonitrile (3 ml) was refluxed under nitrogen for 2 h then cooled to room temperature. The suspension was filtered, washing with dichloromethane and the combined filtrate and washings concentrated to give a red-brown solid (0.147 g). The solid was purified by column chromatography (SiO₂) eluting with 100% DCM and 99:1 DCM/MeOH to give the methyl ester (12) as a beige solid (0.107 g, 79%), mp. 75-7° C. (TLC: R_{F}=0.20 on SiO₂ with 95:5 DCM/MeOH).

¹H nmr (300 MHz, d₆-dmso) δ 2.61, m, CH₂; 2.70, m, CH₂; 3.52, s, H3; 3.58, s, OMe; 3.93, s, SCH₂CO; 6.88, s, H7; 7.67, s, H4; 10.73, s, COOH.

ESI (+ve) m/z 350/352 (M+Na, 90/30%), 328/330 (M+H, 100/30%). ESI (−ve) m/z 326/328 (M−H, 30/10%).

### Example 13

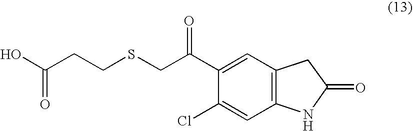

### Preparation of 3-((2-(6-chloro-2-oxo-2,3-dihydro-1H-indol-5-yl)-2-oxoethyl)thio)propanoic acid (13) (Method 1)

The methyl ester (12) (0.051 g, 0.16 mmol) was treated with concentrated hydrochloric acid (2 ml) and heated at reflux briefly (< 1 min) then cooled to room temperature. The suspension was filtered, the solid washed carefully with water and dried under vacuum to give the acid (13) as a light brawn solid (0.041 g, 83%), mp. 203-5° C. (TLC: R_{F}=0.63 on SiO₂ with 8:2 DCM/MeOH).

¹H nmr (300 MHz, d₆-dmso) δ 2.5, obscured, CH₂; 2.66, t (6.6 Hz), CH₂; 3.53, s, H3; 3.92, s, SCH₂CO; 6.88, s, H7; 7.67, s, H4; 10.73, s, NH; 12.23, br s, COOH.

ESI (+ve) m/z 336/338 (M+Na, 10/4%), 314/316 (M+H, 15/4%). ESI (−ve) m/z 312/314 (M−H, 100/35%).

### Preparation of 3-((2-(6-chloro-2-oxo-2,3-dihydro-1H-indol-5-yl)-2-oxoethyl)thio)propanoic acid (13) (Method 2)

5-Chloroacetyl-6-chlorooxindole (1.2 g, 4.8 mmol), 3-mercaptopropionic acid (0.60 g, 0.5 ml, 5.65 mmol) and DMF (5 ml) were added to a 50 ml flask. Diisopropylethylamine (1.8 ml, 10.3 mmol) was added to the reaction mixture with stirring which was continued for 10 h at room temperature under nitrogen. The reaction mixture was then added dropwise with stirring to 200 ml of 10% citric acid solution resulting in the formation of a white precipitate. After cooling in a refrigerator for 4 h the solid material was filtered, washed with water (3×50 ml) and hexane (3×20 ml) then dried under vacuum to give the acid (13) as an off-white solid (1.43 g, 95%), identical to the material prepared by Method 1.

Using Method 2 described above, examples 14-21 were prepared by reaction of either 5-chloroacetyloxindole, 5-chloroacetyl-6-chlorooxindole, 6-chloroacetyl-2-benzoxazolinone or 6-bromoacetyl-2-benzothiazolinone, with 3-mercaptopropionic acid, 6-mercapto-1-hexanol, 1-butanethiol or thioglycolic acid.

### Example 14

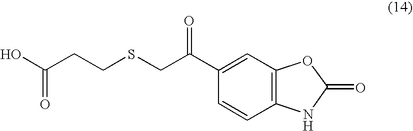

### 3-((2-Oxo-2-(2-oxo-2,3-dihydro-1,3-benzoxazol-6-yl)ethyl)thio)propanoic acid (14)

¹H nmr (400 MHz, d₆-dmso) δ 2.46 (t, 2H); 2.61 (t, 2H); 3.95 (s, 2H); 7.18 (d, 1H); 7.82 (m, 2H).

### Example 15

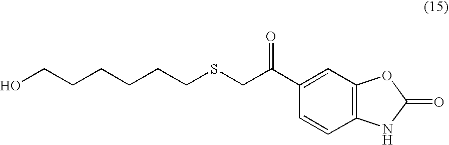

### 6-(((6-Hydroxyhexyl)thio)acetyl)-1,3-benzoxazol-2(3H)-one (15)

¹H nmr (400 MHz, d₆-dmso) δ 1.1-1.5 (m, 8H); 2.42 (m, 4H); 3.89 (s, 2H); 4.3 (t, 1H); 7.15 (d, 1H); 7.8 (m, 2H); 12.1 (s, 1H).

### Example 16

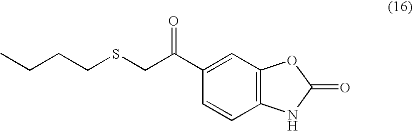

### 6-((Butylthio)acetyl)-1,3-benzoxazol-2(3H)-one (16)

¹H nmr (400 MHz, de-dmso) δ 0.79 (t, 3H); 1.28 (m, 2H); 1.42 (m, 2H); 2.42 (m, 2H); 3.9 (s, 2H); 7.18 (d, 1H); 7.85 (m, 2H).

### Example 17

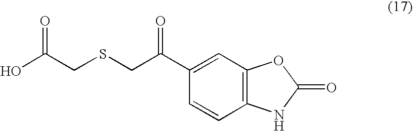

### ((2-Oxo-2-(2-oxo-2,3-dihydro-1,3-benzoxazol-6-yl)ethyl)thio)acetic acid (17)

¹H nmr (400 MHz, d₆-dmso) δ 3.2 (s, 2H); 4.05 (s, 2H); 7.15 (d, 1H); 7.8 (m, 2H).

### Example 18

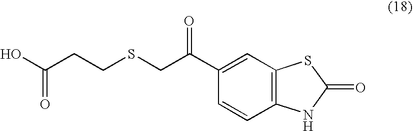

### 3-((2-Oxo-2-(2-oxo-23-dihydro-1,3-benzothiazol-6-yl)ethyl)thio)propanoic acid (18)

¹H nmr (400 MHz, d₆-dmso) δ 2.46 (m, 2H); 2.60 (m, 2H); 3.95 (s, 2H); 7.15 (d, 1H); 7.86 (d, 1H); 8.23 (s, 1H); 12.27 (s, 1H).

### Example 19

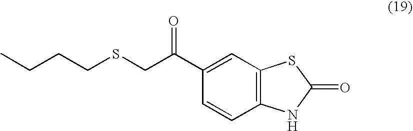

### 6-((Butylthio)acetyl)-1,3-benzothiazol-2(3H)-one (19)

¹H nmr (400 MHz, d₆-dmso) δ 0.79 (m, 3H); 1.26 (m, 2H); 1.43 (m, 2H); 2.42 (m, 2H); 3.87 (s, 2H); 7.1.5 (d, 1H); 7.86 (m, 1H); 8.22 (s, 1H); 12.27 (s, 1H).

### Example 20

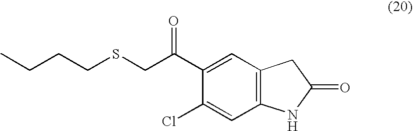

### 5-((Butylthio)acetyl)-6-chloro-1,3-dihydro-2H-indol-2-one (20)

¹H nmr (400 MHz, d₆-dmso) δ 0.79 (t, 3H); 1.25 (m, 2H); 1.42 (m, 210; 2.42 (t, 2H); 3.49 (s, 2H); 3.81 (s, 2H); 6.84 (s, 1H); 7.63 (s, 1H); 10.74 (s, 1H).

### Example 21

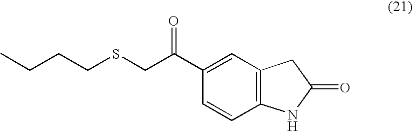

### 5-((Butylthio)acetyl)-1,3-dihydro-2H-indol-2-one (21)

¹H nmr (400 MHz, d₆-dmso) δ 0.79 (t, 3H); 1.25 (m, 2H); 1.43 (m, 2H); 2.42 (t, 2H); 3.50 (s, 2H); 3.83 (s, 2H); 6.85 (d, 1H); 7.77 (s, 1H); 7.83 (d, 1H); 10.75 (s, 1H).

### Example 22

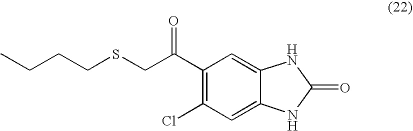

### Preparation of 5-((butylthio)acetyl)-1,3-dihydro-2H-benzimidazol-2-one (22)

1-Butanethiol (647 mg, 7.17 mmol) was dissolved in anhydrous THF (24 ml) and 5-(chloroacetyl)-1,3-dihydro-2H-benzimidazol-2-one (see example 3) (1.497 g, 7.11 mmol) and anhydrous potassium carbonate (4.938 g, 35.7 mmol) were added. The mixture was stirred at room temperature overnight then the reaction mixture was partitioned between ethyl acetate (75 ml) and water (75 ml). The aqueous phase was extracted with ethyl acetate (75 ml) and the combined ethyl acetate extracts were washed with water (2×75 ml) and brine (75 ml), dried over anhydrous magnesium sulfate and filtered. The filtrate was concentrated to approx 30 ml and chilled overnight. The mixture was then filtered and the residue dried under vacuum to give the title compound (1.429 g, 76% yield) as a brown powder, mp 211-213° C.

¹H nmr (400 MHz, d₆-dmso) δ 0.85 (t, J=7.4 Hz, 3H); 1.32 (sextet, J=7.5 Hz, 2H); 1.50 (quintet, J=7.3 Hz, 2H); 2.47-2.53 (resonance obscured by residual ds-dmso); 3.92 (s, 2H); 7.02 (d, J=8.4 Hz, 1H); 7.50 (d, J=1.6 Hz, 1H); 7.68 (dd, J=8.2, 1.8 Hz, 1H); 10.90 (br s, 1H); 11.07 (br s, 1H).

### Example 23

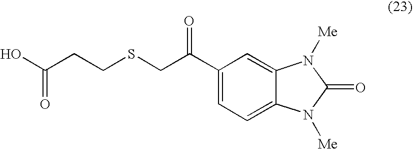

### Preparation of 3-((2-(1,3-dimethyl-2-oxo-2,3-dihydro-1H-benzimidazol-5-yl)-2-oxoethyl)thio) propanoic acid (23)

### (i) 1,3-Dimethyl-1,3-dihydro-2H-benzimidazol-2-one

1,3-Dihydro-2H-benzimidazol-2-one (7.522 g, 56.1 mmol) was dissolved in anhydrous DMF (125 ml) and anhydrous potassium carbonate (46.581 g, 337 mmol) and iodomethane (21 ml, 337 mmol) were added then the mixture stirred at room temperature overnight. The reaction mixture was poured into chloroform (500 ml), filtered and the filtrate was evaporated to dryness. The resultant residue was dissolved in a mixture of ethyl acetate (150 ml) and water (100 ml). The ethyl acetate phase was washed with water (2×100 ml) and brine (100 ml), dried over anhydrous magnesium sulfate and filtered. The filtrate was evaporated to dryness to give the title compound (7.229 g, 79% yield) as a pale yellow solid.

¹H nmr (400 MHz, CDCl₃) δ 3.43 (s, 6H); 6.95-7.01 (m, 2H); 7.08-7.14 (m, 2H).

### (ii) 5-(Chloroacetyl)-1,3-dimethyl-1,3-dihydro-2H-benzimidazol-2-one

Aluminium chloride (13.427 g, 101 mmol) was suspended in DCE (80 ml), cooled in an ice bath and chloroacetyl chloride (6.4 ml, 80 mmol) added dropwise with a glass dropping pipette. The mixture was stirred at 0° C. under nitrogen for 30 min then 1,3-dimethyl-1,3-dihydro-2W-benzimidazol-2-one (6.504 g, 40-1 mmol) was added in portions. The mixture was healed at 55° C. under nitrogen for 2 h, then allowed to cool to room temperature and poured onto ice (200 g). The mixture was filtered and the residue washed with water (100 ml). The filtrate contained two phases which were separated. An attempt was made to dissolve the residue in a mixture of the DCE phase from the filtrate, additional DCE (50 ml) and chloroform (150 ml). The residue only partially dissolved and washing this mixture with water (100 ml) gave an emulsion. The mixture in die separating funnel was filtered and the remaining solid in the separating funnel was suspended in water (3×100 ml) and ethyl acetate (40 ml). Each of the washes was filtered and the residue was dried at the pump and then dried under vacuum over silica gel overnight to give the title compound (7.003 g, 85% yield) as a pink solid.

¹H nmr (400 MHz, d₆-dmso) δ 3.36-3.41 (m, 6H); 5.18 (s, 2H); 7.30 (d, J=8.4 Hz, 1H); 7.76 (d, J=1.2 Hz, 1H); 7.81 (dd, J=8.2, 1.4Hz, 1H).

### (iii) 3-((2-(1,3-Dimethyl-2-oxo-2,3-dihydro-1H-benzimidazol-5-yl)-2-oxoethyl)thio)propanoic acid (23)

3-Mercaptopropionic acid (583 mg, 5.49 mmol) was dissolved in anhydrous DMF (17 ml) and 5-(chloroacetyl)-1,3-dimethyl-1,3-dihydro-2H-benzoimidazol-2r-one (1.298 g, 5.44 mmol) and anhydrous potassium carbonate (3.796 g, 27.5 mmol) were added. The mixture was stirred under nitrogen for 75 min then the reaction mixture was partitioned between ethyl acetate (150 ml) and hydrochloric acid (1M, 80 ml). The aqueous phase was extracted with ethyl acetate (100 ml) and the combined ethyl acetate extracts were washed with water (2×100 ml) and brine (100 ml), dried over anhydrous magnesium sulfate and filtered. The filtrate was evaporated to dryness to give the title compound (1.149 g, 69% yield) as a pale orange solid, mp 174-176° C.

¹H nmr (400 MHz, d₆-dmso) δ 2.54 (t, J=1.2 Hz, 2H); 2.70 (t, J=7.0 Hz, 2H); 3.38 (s, 3H); 3.39 (s, 3H); 4.05 (s, 2H); 7.26 (d, J=8.0 Hz, 1H); 7.76 (d, J=1.6 Hz, 1H); 7.81 (dd, J=8.2, 1.8 Hz, 1H); 12.28 (br s, 1H).

### Example 24

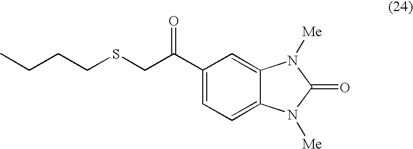

### Preparation of 5-((butylthio)acetyl)-1,3-dimethyl-1,3-dihydro-2H-benzimidazol-2-one

1-Butanethiol (616 mg, 6.83 mmol) was dissolved in anhydrous THF (21 ml) and 5-(chloroacetyl)-1,3-dimethyl-1,3-dihydro-2H-benzimidazol-2-one (1.604 g, 6.72 mmol) and anhydrous potassium carbonate (4.633 g, 33.5 mmol) were added. The mixture was stirred at room temperature for 4 days before the reaction mixture was partitioned between ethyl acetate (60 ml) and water (60 ml). The aqueous phase was extracted with ethyl acetate (60 ml) and the combined ethyl acetate extracts were washed with water (2×60 ml) and brine (60 ml), dried over anhydrous magnesium sulfate and filtered. The filtrate was evaporated to dryness to give an orange oil which solidified on standing. The solid was broken up to give the title compound (1.868 g, 95% yield) as a yellow powder, mp 80-81° C.

¹H nmr (400 MHz, d₆-dmso) δ 0.86 (t, J=6.8 Hz, 3H); 1.32 (sextet, J=7.3 Hz, 2H); 1.51 (quintet, J=7.3 Hz, 2H); 2.49-2.54 (resonance obscured by residual d₅-dmso); 3.37 (s, 3H); 3.39 (s, 3H); 3.98 (s, 2H); 7.25 (d, J=8.4 Hz, 1H); 7.75 (d, J=1.6 Hz, 1H); 7.81 (dd, J=8.2, 1.4 Hz, 1H).

### Example 25

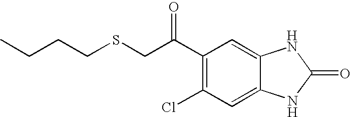

### Preparation of 5-((butylthio)acetyl)-6-chloro-1,3-dihydro-2H-benzimidazol-2-one (25)

### (i) 5-Chloro-6-(chloroacetyl)-1,3-dihydro-2H-benzimidazol-2-one

Aluminium chloride (9.953 g, 74.6 mmol) was suspended in DCE (20 ml) and cooled in an ice bath. Chloroacetyl chloride (4.70 ml, 59.0 mmol) was added dropwise with a glass dropping pipette and the mixture was stirred at 0° C. under nitrogen for 30 min. 5-Chloro-1,3-dihydro-2H-benzimidazol-2-one (5.000 g, 29.7 mmol) was added in portions and the mixture was heated at 55° C. under nitrogen for 3½ h. The mixture was allowed to stand at room temperature under nitrogen overnight, then heated at 55° C. under nitrogen for a further 5½ h. The reaction mixture was allowed to cool to room temperature and poured onto ice (400 g) and filtered. The residue was washed with water (2×100 ml) and dried at the pump. The residue was washed with ethyl acetate (20 ml, 3×40 ml) and dried at die pump to give the title compound (2.631 g, 36% yield) as a dark green powder. The product contained 10 mol % 5-chloro-1,3-dihydro-2H-benzimidazol-2-one.

¹H nmr (400 MHz, d₆-dmso) δ 5.04 (s, 2H); 7.06 (s, 1H); 7.36 (s, 1H); 11.11 (br s, 1H); 11.15 (br s, 1H).

### (ii) 5-((Butylthio)acetyl)-6-chloro-1,3-dihydro-2H-benzimidazol-2-one (25)

1-Butanethiol (478 mg, 5.30 mmol) was dissolved in anhydrous DMF (17 ml) and 5-chloro-6-(chloroacetyl)-1,3-dihydro-2H-benzimidazol-2-one (1.297 g, 5.29 mmol) and anhydrous potassium carbonate (3.666 g, 26.5 mmol) were added. The mixture was stirred under nitrogen for 40 min then the reaction mixture was partitioned between ethyl acetate (100 ml) and hydrochloric acid (3M, 80 ml). The aqueous phase was extracted with ethyl acetate (50 ml) and the combined ethyl acetate extracts were washed with water (50 ml). A further portion of ethyl acetate (100 ml) was added to the ethyl acetate phase and the ethyl acetate extracts were washed with water (50 ml) and brine (50 ml), dried over anhydrous magnesium sulfate and filtered. The filtrate was evaporated to dryness to give a red grey powder (1.441 g, 91% yield). ¹H nmr analysis showed that the product contained 10 mol % unchanged starting material. The crude product was dissolved in anhydrous DMF (15 ml) and a solution of 1-butanethiol (100 mg, 1.11 mmol) in anhydrous DMF (2 ml) was added followed by anhydrous potassium carbonate (759 mg, 5.49 mmol). The mixture was stirred under nitrogen for 60 min before the mixture was partitioned between ethyl acetate (100 ml) and water (100 ml). The aqueous phase was extracted with ethyl acetate (100 ml) and the combined ethyl acetate extracts were washed with water (2×75 ml) and brine (75 ml), dried over anhydrous magnesium sulfate and filtered. The filtrate was evaporated to dryness to give a brown solid which was transferred to a sinter funnel, washed with absolute ethanol (20 ml) and dried at the pump. The residue was dried under vacuum over potassium hydroxide pellets overnight to give die title compound (880 mg, 56% yield) as a pink, powder, mp 199-201° C.

¹H nmr (400 MHz, d₆-dmso) δ 0.84 (t, J=7.2 Hz, 3H); 1.31 (sextet, J=7.4 Hz, 2H); 1.48 (quintet, J=7.4 Hz, 2H); 2.48 (resonance obscured by residual ds-dmso); 3.89 (s, 2H); 7.02 (s, 1H); 7.30 (s, 1H); 11.05 (br s, 2H).

### Example 26

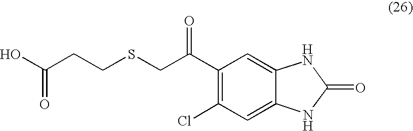

### Preparation of 3-((2-(6-chloro-2-oxo-2,3-dihydro-1H-benzimidazol-5-yl)-2-oxoethyl)thio)propanoic acid (26)

3-Mercaptopropionic acid (566 mg, 5.33 mmol) was dissolved in anhydrous DMF (17 ml) and 5-chloro-6-(chloroacetyl)-1,3-dihydro-2H-benzimidazol-2-on B (1.300 g, 5.30 mmol) and anhydrous potassium carbonate (3.714 g, 26.9 mmol) were added. The mixture was stirred under nitrogen for 35 min then the reaction mixture was partitioned between ethyl 1 acetate (100 ml) and water (150 ml). Emulsions prevented the separation of the phases and hydrochloric acid (1M, 80 ml) was carefully added. The phases were separated and the aqueous-phase was extracted with ethyl acetate (100 ml, 50 ml). The combined ethyl acetate extracts were washed with water (2×100 ml) and brine (100 ml), dried over anhydrous magnesium sulfate, and filtered. The filtrate was evaporated to dryness to give a dark green powder.

The crude product was partitioned between ethyl acetate (150 ml) and a 5% sodium hydrogen carbonate solution (200 ml). The ethyl acetate phase was extracted with water (100 ml) and the combined aqueous phases were washed with ethyl acetate (100 ml), acidified with hydrochloric acid (3M, 50 ml) and extracted with ethyl acetate (300 ml, 2×100 ml). There was a significant quantity of a pale brown solid that did not dissolve. The aqueous phase containing the emulsion was filtered and dried at the pump to give the title compound (447 mg, 27% yield) as a cream solid.

The ethyl acetate extracts were evaporated to dryness to give a green solid and the residue partitioned between ethyl acetate (150 ml) and a 5% sodium hydrogen carbonate solution (200 ml). The aqueous phase was acidified with hydrochloric acid (3M, 50 ml) and the resultant suspension washed with ethyl acetate (50 ml). The combined aqueous and ethyl acetate phases were filtered and the residue was washed with water (2×50 ml) and dried at the pump to give the title compound (579 mg, 35% yield) as a pale green solid.

The two batches of 3-((2-(6-chloro-2-oxo-2,3-dihydro-1H-benzimidazol-5-yl)-2-oxoethyl)thio)propanoic acid were dried under vacuum over silica gel overnight. Both samples had a similar appearance after drying and the two samples were combined to give the title compound (1.013 g, 61% yield) as a pale beige powder, mp 186-187° C.

¹H nmr (400 MHz, d₆-dmso) δ 2.51 (resonance obscured by residual d₅-dmso); 2.68 (t, J=7.2 Hz, 2H); 3.97 (s, 2H); 7.02 (s, 1H); 7.31 (s, 1H); 11.03 (br s, 1H); 11.09 (br s, 1H); 12.29 (br s, 1H).

### Example 27

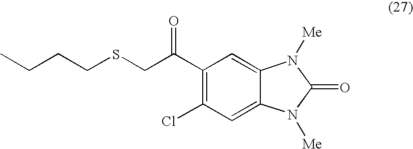

### Preparation of 5-((butylthio)acetyl)-6-chloro-1,3-dimethyl-1,3-dihydro-2H-benzimidazol-2-one (27)

### (i) 5-Chloro-1,3-dimethyl-1,3-dihydro-2H-benzoimidazol-2-one

5-Chloro-1,3-dihydro-2H-benzimidazol-2-one (7.040 g, 41.8 mmol) was dissolved in anhydrous DMF (100 ml), anhydrous potassium carbonate (34.707 g, 251 mmol) and iodomethane (15.5 ml, 249 mmol) were added and the mixture stirred at room temperature overnight. The reaction mixture was poured into chloroform (400 ml) and mixed well then filtered and the filtrate evaporated to dryness. The resultant residue was dissolved in a mixture of ethyl acetate (500 ml) and water (200 ml), the ethyl acetate phase was washed with water (2×100 ml) and brine (100 ml), dried over anhydrous magnesium sulfate and filtered. The filtrate was evaporated to dryness to give the title compound (7.163 g, 87% yield) as a brown powder.

¹H nmr (400 MHz, CDCl₃) δ 3.37-3.42 (m, 6H); 6.87 (d, 3-8.0 Hz, 1H); 6.97 (d, J=1.6 Hz, 1H); 7.07 (dd, J=8.2, 1.8 Hz. 1H).

### (ii) 5-Chloro-6-(chloroacetyl)-1,3-dimethyl-1,3-dihydro-2H-benzoimidazol-2-one

Aluminium chloride (8.589 g, 64.4 mmol) was suspended in DCE (17 ml) and cooled in an ice bath. Chloroacetyl chloride (4.05 ml, 50.8 mmol) was added dropwise with a glass dropping pipette and the mixture was stirred at 0° C. under nitrogen for 30 min. 5-Chloro-1,3-dimethyl-1,3-dihydro-2H-benzimidazol-2-one (5.010 g, 25.5 mmol) was added in portions and the mixture was heated at 55° C. under nitrogen for 3 h. The mixture was allowed to cool to room temperature and poured onto ice (200 g) then filtered. The residue was washed with water (3×100 ml), dried at the pump and then dried under vacuum over silica gel to give the title compound (5.573 g, 80% yield) as a brown powder.

¹H nmr (400 MHz, d₆-dmso) δ 3.33-3.37 (m, 6H); 5.09 (s, 2H); 7.45 (s, 1H); 7.69 (s, 1H).

### (iii) 5-((Butylthio)acetyl)-6-chloro)-1,3-dimethyl-1,3-dihydro-2H-benzoimidazol-2-one (27)

1-Butanethiol (533 mg, 5.91 mmol) was dissolved in anhydrous THF (19 ml) and 5-chloro-6-(chloroacetyl)-1,3-dimethyl-1,3-dihydro-2H-benzimidazol-2-one (1.598 g, 5.85 mmol) and anhydrous potassium carbonate (4.071 g, 29.5 mmol) were added followed by anhydrous DMF (1.0 ml). The mixture was stirred at room temperature for 5 days then the reaction mixture was partitioned between ethyl acetate (60 ml) and water (60 ml). The aqueous phase was extracted with ethyl acetate (60 ml) and the ethyl acetate extracts were washed with water (2×60 ml) and brine (60 ml), dried over anhydrous magnesium sulfate and filtered. The filtrate was evaporated to dryness and the resultant residue purified by bulb-to-bulb distillation (250° C./0.57 mbar) to give the title compound (1.037 g, 54% yield) as a pale yellow solid, mp 66-68° C.

¹H nmr (400 MHz, d₆-dmso) δ 0.85 (t, J=7.4 Hz, 3H); 1.32 (sextet, J=7.3 Hz, 210; 1-49 (quintet, J=7.4 Hz, 2H); 2.48-2.53 (resonance obscured by residual d₅-dmso); 3.34-3.37 (m, 6H); 3.95 (s, 2H); 7.40 (s, 1H); 7.62 (s, 1H).

### Example 28

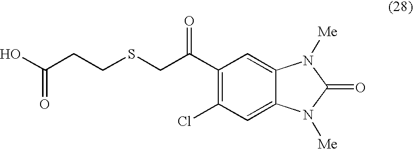

### Preparation of 3-((2-(6-chloro-1,3-dimethyl-2-oxo-2,3-dihydro-1H-benzimidazol-5-yl)-2-oxoethyl)thio)propanoic acid (28)

3-Mercaptopropionic acid (593 mg, 5.59 mmol) was dissolved in anhydrous DMF (17 ml) and 5-chloro-6-(chloroacetyl)-1,3-dimethyl-1,3-dihydro-2W-benzimidazol-2-one (1.498 g, 5.48 mmol) and anhydrous potassium carbonate (3.784 g, 27.4 mmol) were added. The mixture was stirred under nitrogen for 35 min then partitioned between ethyl acetate (100 ml) and hydrochloric acid (1M, 80 ml). The aqueous phase was extracted with ethyl acetate (50 ml) and the combined ethyl acetate extracts were washed with water (2×50 ml) and brine (50 ml), dried over anhydrous magnesium sulfate and filtered. The filtrate was evaporated to dryness to give the title compound (1.797 g, 96% yield) as a brown powder, mp 148-150° C.

¹H nmr (400 MHz, d₆-dmso) δ 2.53 (resonance obscured by residual ds-dmso); 2.69 (t, J=7.2Hz, 2H); 3.35 (s, 3H); 3.36 (s, 3H); 4.02 (s, 2H); 7.41 (s, 1H); 7.63 (s, 1H); 12.30 (br s, 1H).

### Example 29

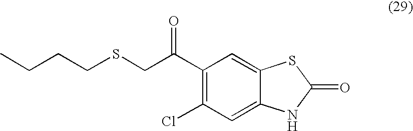

### Preparation of 6-((butylthio)acetyl)-5-chloro-1,3-benzothiazol-2(3H)-one (29)

### (i) 5-Chloro-6-(chloroacetyl)-1,3-benzothiazol-2(3H)-one

Anhydrous DMF (8.2 ml) was added dropwise to aluminium chloride (40.846 g, 306 mmol) with stirring (Caution: exothermic). The mixture was stirred until an even slurry formed then 5-chloro-2-benzothiazolone (7.020 g, 37.8 mmol) was added in portions. The mixture was heated at 70° C. under nitrogen then bromoacetyl bromide (5.6 ml, 64 mmol) was added and the mixture heated at 70° C. under nitrogen for 25 h. The mixture was allowed to stand at room temperature under nitrogen overnight then was poured onto ice (200 g), stirred for 1 h and filtered. The residue was washed with water (2×100 ml) and dried at the pump then washed with ethyl acetate (3×25 ml) and dried at die pump to give the title compound (4.779 g, 41% yield) as a dark green powder. ¹H nmr analysis showed that the product also contained 6-(bromoacetyl)-5-chloro-1,3-benzothiazol-2(3H)-one (23 mol %) and an unidentified impurity (14 mol %).

¹H nmr (400 MHz, d₆-dmso) δ 5.04 (s, 2H); 7.22 (s, 1H); 8.17 (s, 1H); 12.41 (br s, 1H). Bromoacetyl impurity: δ 4.84 (s, 2H); 7.22 (s, 1H); 8.19 (s, 1H); 12.41 (br s, 1H), Unidentified impurity: δ 7.27 (s, 1H); 8.08 (s, 1H); 12.17 (br s, 1H).

### (ii) 6-((Butylthio)acetyl)-5-chloro-1,3-benzothiazol-2(3H)-one (29)

1-Butanethiol (557 mg, 6.18 mmol) was dissolved in anhydrous DMF (19 ml). A mixture of 5-chloro-6-(chloroacetyl)-1,3-benzothiazol-2(3H)-one (63 mol %), 6-(bromoacetyl)-5-chloro-1,3-benzothiazol-2(3H)-one (23 mol %) and an unidentified impurity (14 mol %) (1.610 g, 6.14 mmol) and anhydrous potassium carbonate (4.236 g, 30.6 mmol) were added. The mixture was stirred at room temperature under nitrogen for 105 min then was partitioned between ethyl acetate (150 ml) and hydrochloric acid (1M, 80 ml). The aqueous phase was extracted with ethyl acetate (50 ml) and the combined ethyl acetate extracts were washed with water (2×75 ml) and brine (75 ml), dried over anhydrous magnesium sulfate, and filtered. The filtrate was evaporated to dryness and the resultant residue dried under high vacuum (1.00° C./0.8 mbar for 5 min) to give a dark brown solid (1.317 g). A portion of the crude product (325 mg) was dissolved in ethyl acetate (20 ml) and silica gel 60 (1.5 g) was added and the mixture evaporated to dryness and purified by flash chromatography over silica gel 60 (eluent: 30% ethyl acetate/petroleum spirits (5×20 ml fractions), packing height: 20 cm, column diameter: 1 cm for 19 cm, then 2.5 cm). The fractions containing the first major band (Rf 0.40, eluent: 30% ethyl acetate/petroleum spirits, fractions 2-4) were combined and evaporated to dryness. The residue was dried under high vacuum (100° C./0.8 mbar for 5 min) to give the title compound (248 mg, 13% yield) as an orange melt, mp 102.5-115.0° C. ¹H nmr analysis showed that the product contained the unidentified impurity (20 mol %) that was present in the starting material.

¹H nmr (400 MHz, d₆-dmso) δ 0.85 (t, J=7.4 Hz, 3H); 1.31 (sextet, J=7.41 Hz, 2H); 1.48 (quintet, J=7.4 Hz, 2H); 2.49 (resonance obscured by residual d₅-dmso); 3.89 (s, 2H); 7.19 (s, 1H); 8.13 (s, 1H); 12.29 (br s, 1H). Impurity present in starting material: δ 7.27 (s, 1H); 8.08 (s, 1H).

### Example 30

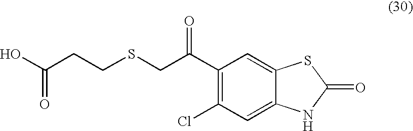

### Preparation of 3-((2-(5-chloro-1-oxo-2,3-dihydro-1,3-benzothiazol-6-yl)-2-oxoethyl)thio)propanoic acid (30)

### (i) 6-(Bromoacetyl)-5-chloro-1,3-benzothiazol-2(3H)-one

Anhydrous DMF (8.2 ml) was added dropwise to aluminium chloride (40.556 g, 304 mmol) with stirring (Caution: exothermic). The mixture was stirred until an even slurry formed then 5-chloro-2-benzothiazolone (7.030 g, 37.9 mmol) was added in portions. The mixture was heated at 70° C. under nitrogen and bromoacetyl bromide (5.6 ml, 64 mmol) added and the mixture heated at 70° C. under nitrogen for 7½ h. The mixture was allowed to stand at room temperature under nitrogen overnight then the mixture was poured onto ice (200 g), stirred for 1 h and filtered. The residue was washed with water (2×100 ml) and dried at the pump. The residue was then washed with ethyl acetate (2×40 ml) and dried at tire pump to give the title compound (3.150 g, 27% yield) as a tan powder. ¹H nmr analysis showed that the product also contained 5-chloro-2-benzothiazolone (33 mol %) and 5-chloro-6-(chloroacetyl)-1,3-benzothiazol-2(3H)-one (32 mol %).

¹H nmr (400 MHz, d₆-dmso) δ 4.84 (s, 2H); 7.22 (s, 1H); 8.19 (s, 1H); 12.41 (br s, 1H). Starting material; δ 7.12 (d, J=2.0 Hz, 1H); 7.19 (dd, J=8.4, 2.0 Hz. 1H); 7.62 (d, J=8.4 Hz, 1H); 12.06 (br s, 1H). Chloroacetyl impurity; δ 5.04 (s, 2H); 7.22 (s, 1H); 8.17 (s, 1H); 12.41 (br s, 1H).

### (ii) 3-((2-(5-chloro-2-oxo-2,3-dihydro-1,3-benzothiazol-6-yl)-2-oxoethyl)thio)propanoic acid (30)

3-Mercaptopropionic acid (566 mg, 5.33 mmol) was dissolved in anhydrous DMF (17 ml) and a mixture of 6-(bromoacetyl)-5-chloro-1,3-benzothiazol-2(3H)-one (35 mol %), 5-chloro-6-(chloroacetyl)-1,3-benzothiazol-2(3H)-one (32 mol %) and 5-chloro-2-benzothiazolone (33 mol %) (1.999 g, 5.31 mmol based on available bromoacetyl and chloroacetyl compounds) and anhydrous potassium carbonate (3.707 g, 26.8 mmol) were added. The mixture was stirred under nitrogen for 45 min then the reaction mixture was partitioned between ethyl acetate (150 ml) and hydrochloric acid (1M, 80 ml). The aqueous phase was extracted with ethyl acetate (50 ml) and the combined ethyl acetate extracts were washed with water (2×100 ml) and extracted with a 5% sodium hydrogen carbonate solution (200 ml) and water (50 ml). These extracts were acidified with hydrochloric acid (3M, 50 ml) and filtered and the residue was dried under vacuum over silica gel to give the title compound (1.105 g, 63% yield based on available bromoacetyl and chloroacetyl compounds) as a pale yellow solid, mp 195-197° C.

¹H nmr (400 MHz, d₆-dmso) δ 2.52 (resonance obscured by residual ds-dmso); 2.67 (t, J=7.0 Hz, 2H); 3.96 (s, 2H); 7.19 (s, 1H); 8.13 (s, 1H); 12.32 (br s, 2H).

### Example 31

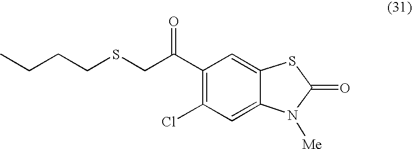

### Preparation of 6-((butylthio)acetyl)-5-chloro-3-methyl-1,3-benzothiazol-2(3H)-one (31)

### (i) 5-Chloro-3-methyl-1,3-benzothiazol-2(3H)-one

5-Chloro-2-benzothiazolone (5.010 g, 27.0 mmol) was dissolved in anhydrous DMF (60 ml) and anhydrous potassium carbonate (11.248 g, 81.4 mmol) and iodomethane (5.05 ml, 81.1 mmol) were added and the mixture stirred at room temperature overnight. The reaction mixture was poured into chloroform (240 ml) and filtered and the filtrate was evaporated to dryness. The resultant residue was dissolved in a mixture of ethyl acetate (300 ml) and water (200 ml) then the phases were separated. The ethyl acetate phase was washed with water (2×100 ml) and brine (100 ml), dried over anhydrous magnesium sulfate, and filtered. The filtrate was evaporated to dryness to give the title compound (5.078 g, 94% yield) as a beige powder.

¹H nmr (400 MHz, CDCl₃) δ 3.44 (s, 3H); 7.05 (d, J=1.6 Hz, 1H); 7.16 (dd, J=8.4, 2.0 Hz, 1H); 7.34 (d, J=8.4 Hz, 1H).

### (ii) 6-(Bromoacetyl)-5-chloro-3-methyl-1,3-benzothiazol-2(3H)-one

Anhydrous DMF (3.0 ml) was added dropwise to aluminium chloride (14.768 g, 111 mmol) with stirring (Caution: exothermic) and the mixture was stirred until an even slurry had formed. 5-Chloro-3-methyl-1,3-benzothiazol-2(3H)-one (2.724 g, 13.6 mmol) was added in portions then the mixture was heated at 70° C. under nitrogen. Bromoacetyl bromide (2.0 ml, 23 mmol) was added and heating continued at 70° C. under nitrogen for a further 6 h. The mixture was allowed to stand at room temperature under nitrogen overnight then poured onto ice (200 g), stirred for 1 h and filtered. The residue was washed with water (2×100 ml) and dried at the pump. The residue was then washed with ethyl acetate (2×20 ml) and dried at the pump to give the title compound (2.538 g, 58% yield) as a red-grey solid. ¹H nmr analysis showed that the product contained 6-(chloroacetyl)-5-chloro-3-methyl-1,3-benzothiazol-2(3H)-one (23 mol %) and unchanged starting material (6 mol %).

¹H nmr (400 MHz, ds-dmso) δ 3.44 (s, 3H); 4.85 (s, 2H); 7.62 (s, 1H); 8.24 (s, 1H). Chloroacetyl impurity: δ 3.44 (s, 3H); 5.05 (s, 2H); 7.62 (s, 1H); 8.22 (s, 1H).

### (iii) 6-((Butylthio)acetyl)-5-chloro-3-methyl-1,3-benzothiazol-2(3H)-one (31)

1-Butanethiol (607 mg, 6.73 mmol) was dissolved in anhydrous DMF (20 ml) and 6-anhydrous potassium carbonate (4.375 g, 31.7 mmol) were added. The mixture was stirred under nitrogen for 105 min then the reaction mixture was partitioned between ethyl acetate (100 ml) and hydrochloric acid (1M, 80 ml). The aqueous phase was extracted with ethyl acetate (50 ml) and the ethyl acetate extracts were washed with water (2×75 ml) and brine (75 ml), dried over anhydrous magnesium sulfate, and filtered. The filtrate was evaporated to dryness to give a brown oil that was purified by bulb-to bulb distillation (235° C./0.80 mbar) to give the title compound (1.328 g, 65% yield) as a brown oil.

¹H nmr (400 MHz, d₆-dmso) δ 0.83 (t, J=7.2 Hz, 3H); 1.29 (sextet, J=7.5 Hz, 2H); 1.47 (quintet, J=7.4 Hz, 2H); 2.47 (resonance obscured by residual d₅-dmso); 3.42 (s, 3H); 3.89 (s, 2H); 7.56 (s, 1H); 8.16 (s, 1H).

### Example 32

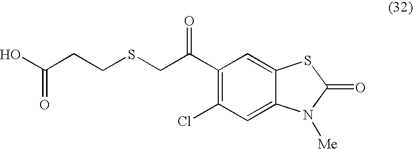

### Preparation of 3-((2-(5-chloro-3-methyl-2-oxo-2,3-dihydro-1,3-benzothiazol-6-yl)-2-oxoethyl)thio)propanoic acid (32)

3-Mercaptopropionic acid (515 mg, 4.85 mmol) was dissolved in anhydrous DMF (15 ml) and 6-(bromoacetyl)-5-chloro-3-methyl-1,3-benzothiazol-2(3H)-one (1.551 g, 4.84 mmol) and anhydrous potassium carbonate (3.595 g, 26.0 mmol) were added. The mixture was stirred under nitrogen for 40 min then the reaction mixture was partitioned between ethyl acetate (150 ml) and hydrochloric acid (1M, 80 ml). The aqueous phase was extracted with ethyl acetate (50 ml) and the combined ethyl acetate extracts were washed with water (2×100 ml), and extracted with a 5% sodium hydrogen carbonate solution (200 ml) and water (50 ml). These extracts were acidified with hydrochloric acid (3M, 50 ml) causing a brown oil to precipitate. The mixture was extracted with ethyl acetate (100 ml, 50 ml) and the ethyl acetate extracts were washed with brine (75 ml), dried over anhydrous magnesium sulfate and filtered. The filtrate was evaporated to dryness to give the title compound (1.476 g 88% yield) as a cream powder, mp 120-121° C.

¹H nmr (400 MHz, dg-dmso) δ 2.53 (resonance obscured by residual ds-dmso); 2.67 (t, J=7.0 Hz, 2H); 3.43 (s, 3H); 3.97 (s, 2H); 7.58 (s, 1H); 8.19 (s, 1H); 12.31 (br s, 1H).

### BIOLOGY EXAMPLES

### Example 1

### Interaction of Compounds with MIF Proteins Detected by Biacore Analysis

### Methods

The interaction of compounds with MIF protein was characterized by Surface Plasmon Resonance (SPR) analysis using an S51 (Biacore International AB) automated small molecule biosensor assay system. Recombinant MIF protein was immobilized on a carboxymethyl dextran biosensor chip using amine coupling chemistry. Compound binding to the immobilized MIF protein was measured at 11 concentrations up to 100 uM (in duplicate), with corrections for the DMSO used as a solvent at a final concentration of 5%. The change in SPR output relative to that of a control underivatized reference spot was recorded over time. The affinity and stoichiometry of interaction was calculated using steady state and/or kinetics evaluation methods with software supplied by the manufacturer.

### Results

The results listed in Table 1 summarize the interaction of Compounds 4, 13, and 19 with immobilized recombinant MIF protein. The compounds bind to MIF with equilibrium dissociation constant (K_{D}) values in the low micromolar range. The predicted stoichiometry of the compound: MIF trimers were determined to be 1:1.

TABLE 1
Summary of affinity and kinetic constants for compound binding to
immobilized MIF.

| | | | | | Predicted |
|---|---|---|---|---|---|
| | | | | | Stoichiometry of |
| | Steady | | | | Complex |
| | State | Kinetics Method | | | Molecules |
| Compound | Method | Kd (uM) | Ka (10³ M⁻¹s⁻¹) | Kd (10⁻³ s⁻¹) | bound/MIF trimer |
| 4 | 5.1 | | | n.d. | 1:1 |
| 13 | 10 ± 1.4 | 2.5 ± 1.2 | 0.6 ± 0.2 | 1.3 ± 0.3 | 1:1 |
| 19 | 27 ± 2.3 | 3.6 ± 1.0 | 2.2 ± 1.0 | 6.2 ± 1.2 | 1:1 |

### Example 2

### In Vitro Assay of MIF Antagonism: Inhibition of LPS-Induced Production of IL-6 in RAW264.7 Macrophages by Compounds

MIF is an important factor in the innate immune response to toxins such as the bacterial endotoxin lipopolysaccharide (LPS). Notably, endogenous MIF activity is required for expression of the LPS receptor toll-like receptor-4⁽¹²⁾. A compound with the ability to inhibit the biological activity of MIF would therefore inhibit the activation of cytokine production by macrophages in response to LPS.

### Methods

The RAW264.7 mouse macrophage cell line was propagated in DMEM/10% foetal calf serum (FCS) at 37° C. in 5% CO₂. 24 hr prior to assay cells were seeded in 96-well tissue culture plates. Cells were allowed to adhere for 4 hr prior to transfer to DMEM/0.5% FCS for 18 hr. Cells were then treated with 50 uM compound in DMSO for 30 min prior to stimulation for 4 hr with 100 ng/ml LPS. Cell culture supernatants were then collected from each well and assayed for IL-6 levels by ELISA (R&D Systems) according to the manufacturer's instructions.

### Results

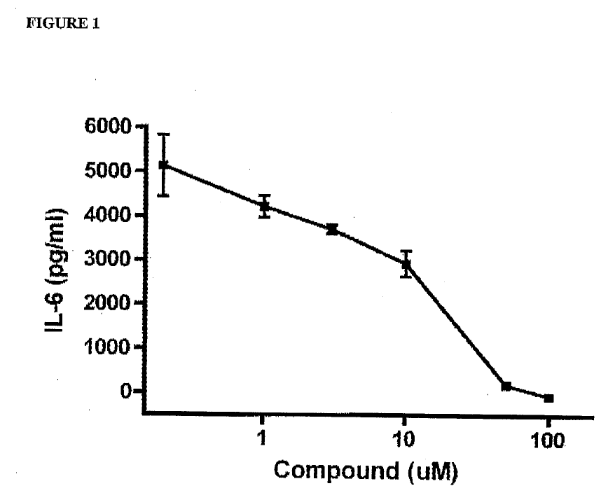

FIG. 1 shows that Compound 19 treatment induces a dose-dependent inhibition of LPS-induced IL-6 production when RAW264.7 cells are pre-treated with up to 100 μM concentration of compound and the samples analysed for IL-6 production as described above. The IC50 value for the compound was determined to be 20 uM.

Table 2 shows the % inhibition of IL-6 production induced by 50 uM compound treatment relative to LPS+DMSO control levels (with basal levels of IL-6 in die absence of LPS subtracted). The compounds induce marked decreases in IL-6 production consistent with antagonism of endogenous MIF.

TABLE 2
Inhibition of LPS-induced IL-6 production in RAW264.7 cells

| Compound | % Inhibition |
|---|---|
| (50 uM) | of IL-6 production |
| 1 | 13 ± 23 |
| 4 | 5 ± 49 |
| 8 | 42 ± 11 |
| 10 | 25 |
| 11 | 48 ± 24 |
| 12 | 60 + 11 |
| 13 | 32 ± 30 |
| 15 | 42 ± 40 |
| 16 | 12 ± 63 |
| 17 | 8 ± 90 |
| 18 | 49 ± 34 |
| 19 | 82 ± 13 |
| 20 | 59 ± 11 |
| 21 | 41 ± 54 |

### Example 3

### In Vitro Assay of MIF Antagonism: Inhibition of Interleukin-1 Induction of Cycloxygenase-2 Expression in S112 Human Dermal Fibroblasts by Compounds

The activity of compounds was studied in a bioassay for MIF-dependent cytokine effects of human S112 dermal fibroblasts. In these cells the induction of the expression of cyclooxygenase-2 (COX-2) protein by interleukin 1 (IL-1) is dependent upon the presence of endogenous MIF⁽¹³⁾. The expression of COX2 proteins is therefore sensitive to depiction of endogenous MIF by neutralizing antibody, gene knockout of targeting with small molecule inhibitors. A compound with the ability to inhibit the biological activity of MIF would therefore inhibit die activation of COX2 expression hi response to IL-1.

### Methods

S112 human dermal fibroblasts were propagated in RPMI/10% foetal calf serum (FCS). Prior to experimentation, cells were seeded at 10⁵ cells/ml in RPMI/0.1% BSA for 18 hours. Cells were treated with recombinant human IL-1 (0.1 ng/ml) and with each compound at concentrations ranging up to 100 μM. A control was treated only with recombinant human TL-1 (0.1 ng/ml) and vehicle (DMSO). After 6 hours, cells were collected and intracellular COX-2 protein determined by permeabilisation flow cytometry. Cells permeabilised with 0.1% saponin were sequentially labelled with a mouse anti-human COX-2 monoclonal antibody and with sheep-anti-mouse F(ab)2 fragment labelled with fluoroscein isothiocyanate. Cellular fluorescence was determined using a flow cytometer. At least 5000 events were counted for each reading, each of which was performed in duplicate, and the results expressed in mean fluorescence intensity (MFI) after subtraction of negative control-labelled cell fluorescence.

### Results

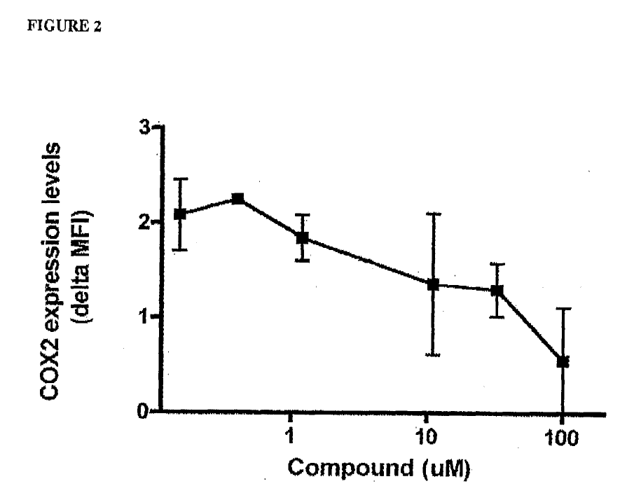

FIG. 2 shows treatment with Compound 2 induces a dose-dependent inhibition of IL-1 induced COX-2 expression when S112 cells are treated with up to 100 μM concentration of compound and the samples analysed for COX2 expression as above. The results show significant and dose-dependent reductions in COX2 expression levels consistent with antagonism of MIF activity.

### Example 4

### In Vivo Assay of MIF Antagonism: Endotoxic Shock

The activities of compounds were studied in the murine endotoxic shock model. This model has been previously shown to be dependent on MIF⁽¹⁴⁾. Administration of a compound which inhibits the cytokine activity of MIF would be expected to result hi a reduction in serum level s of the pro-inflammatory cytokine TNF.

### Methods

Endotoxaemia was induced by intra-peritoneal Injection of C57Bl/6j mice with lipopolysaccharide (LPS) (1 mg/kg) in 200 μl saline. Animals were treated with either a saline solution (control) only, or LPS with vehicle or compounds B1 and A3 in vehicle at doses of 10, 1 and 0.1 mg/kg body weight, administered by intra-peritoncal injection at 24 hours and 1 hour before intra-peritoneal LPS injection. After 1 hour mice were humanely killed by CO₂ inhalation then neck dislocation. Serum was obtained from blood obtained by cardiac puncture prior to death and measured for TNF levels by ELISA according to the manufacturer's instructions.

### Results

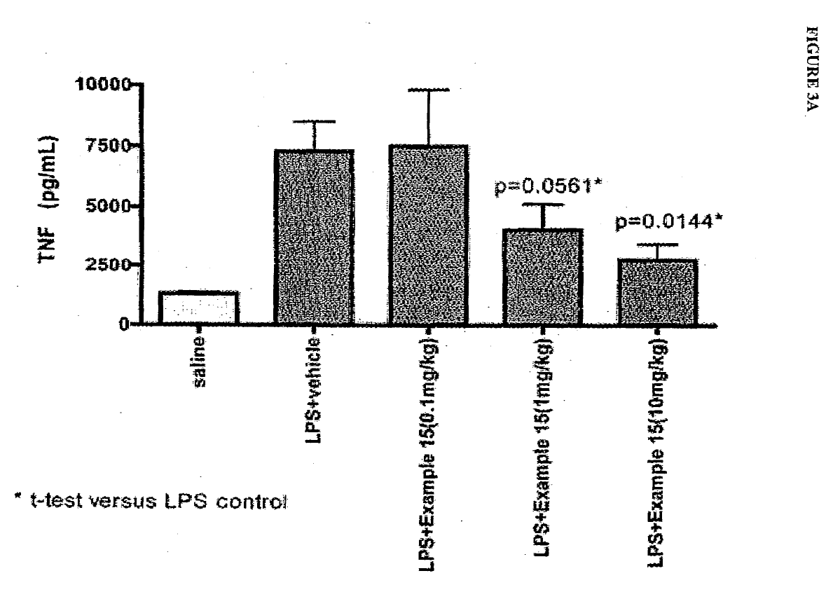

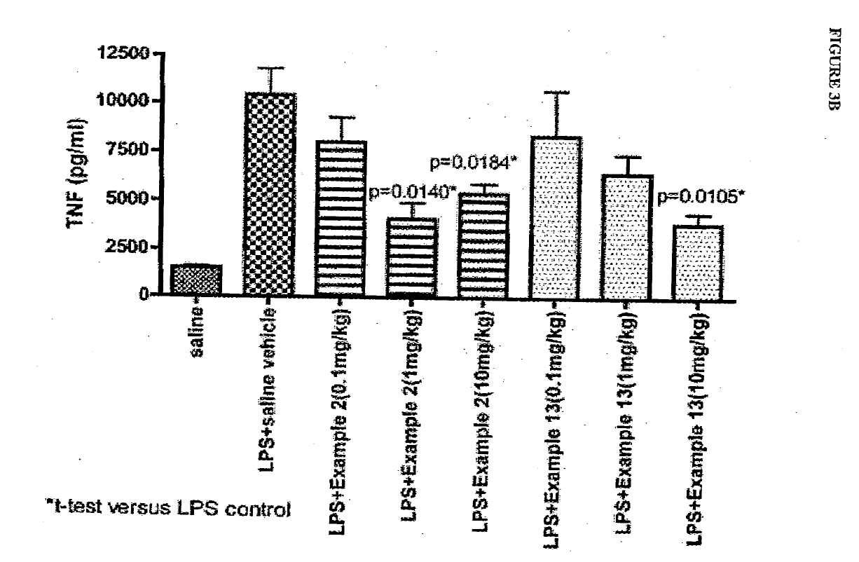

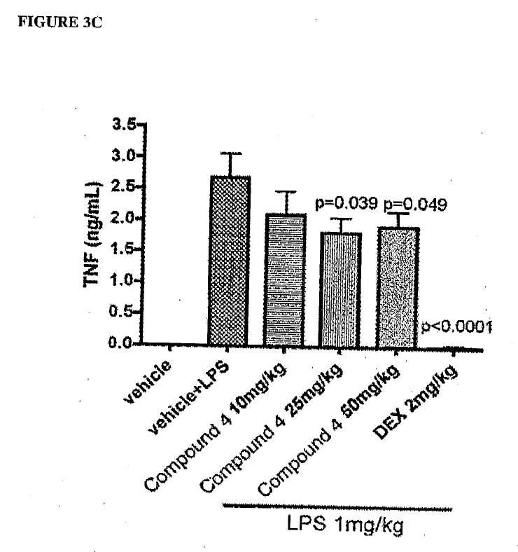

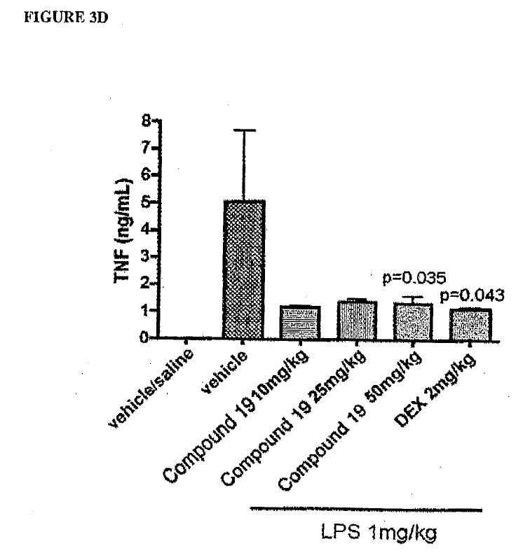

The results in FIG. 3 show that treatment of mice with compounds 15 (FIG. 3A), compounds 2 and 13 (FIG. 3B), compound 4 (FIG. 3C), and compound 19 (FIG. 3D) results in a significant dose-dependent suppression of LPS-induced serum TNF levels in the endotoxic shock model described above.

### Example 5

### Inhibition of MIF Tautisomerase Activity

MIF protein has the ability in vitro to catalyze the tautisomerization of dopachrome⁽¹⁵⁾. The tautomerase activity of MIF is unique, as is the structure and sequence of the section of MIF responsible for this phenomenon, suggesting that small molecules binding to or docking hi this site would be specific for MIF. The relevance of this enzymatic activity to the development of inhibitors of the cytokine and biological activity of MIF is that demonstration of inhibition of tautisomerase activity is a demonstration that a given compound has a direct physical interaction with MIF.

### Methods

Recombinant human MIF protein was pre-incubated with compounds as indicated prior to the addition of L-dopachrome substrate. Tautomerase activity was determined by measurement of die decrease in absorbance at 475 nm after 2 min. The maximum tautisomerase activity detected was recorded as 100%, and the inhibition of this activity at either 50 mM or 100 mM concentration of compounds determined.

### Results

Many compounds were determined to bind to MIF via demonstration of the ability to inhibit the tautisomerase activity of MIF, as shown in Table 3. Values shown are the mean±standard deviation of 2-4 experiments.

TABLE 3
Inhibition of the tautisomerase activity of MIF by selected examples

| | | % Inhibition @ | % Inhibition @ |
|---|---|---|---|
| Compound | Structure | 50 uM | 100 uM |
| 27 | 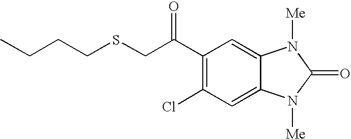 | 28 ± 17 | 44 ± 12 |
| 29 | 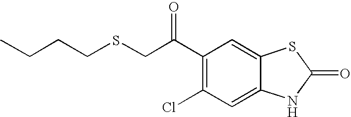 | 24 ± 1 | 42 ± 9 |
| 24 | 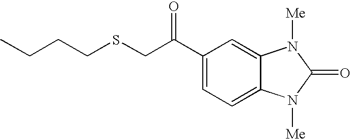 | 15 ± 6 | 34 ± 12 |
| 30 | 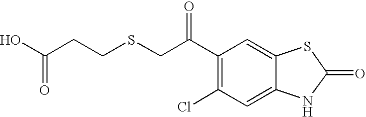 | 12 ± 11 | 27 ± 16 |
| 28 | 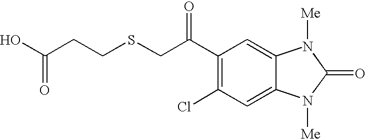 | 12 ± 6 | 27 ± 6 |
| 25 | 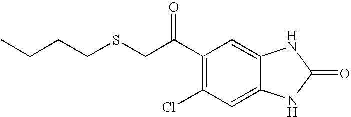 | 4 ± 3 | 12 ± 4 |
| 32 | 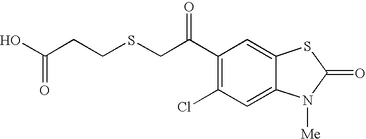 | 4 ± 1 | 11 ± 4 |
| 22 | 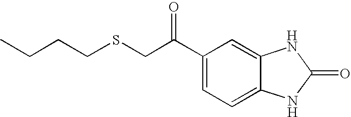 | 4 ± 6 | 4 ± 7 |
| 31 | 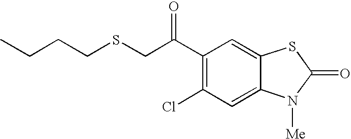 | 2 ± 3 | 8 ± 8 |
| 26 | 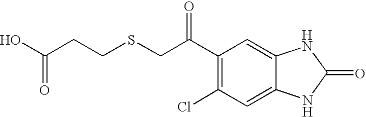 | 2 ± 3 | 4 ± 6 |
| 23 | 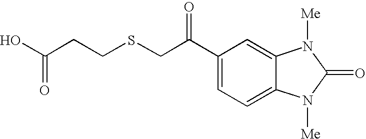 | 0 | 3 ± 2 |
| 19 | 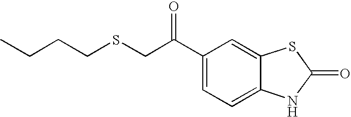 | 9 ± 4 | 17 ± 8 |

### Example 6

Delayed-type hypersensitivity reactions, which are initiated by T lymphocyte responses to recall antigens and mediated by many cell types including macrophages, are known to be dependent on the cytokine or biological activity of MIF^{(16,17)}. For example, an anti-MIF monoclonal antibody suppresses delayed-type hypersensitivity reactions in vivo to methylated bovine scrum albumin (mBSA) injected into the skin of animals preimmunised with mBSA⁽¹⁶⁾. A compound inhibiting die cytokine or biological function of MIF might be expected to inhibit delayed-type hypersensitivity reactions in vivo.

### Methods

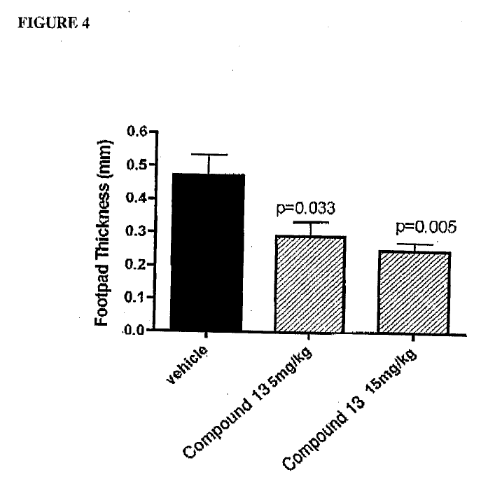

Mice were immunised on day 0 with 200 μg of methylated BSA (mBSA; Sigma Chemical Co., Castle Hill, Australia) emulsified in 0.2 ml of Freund's complete adjuvant (CFA; Sigma) injected subcutaneously in the flank skin. At day 7, mice were given 100 μg mBSA in 0.1 ml CFA by intradermal injection at the base of the tail. Mice were challenged on day 27 following first immunisation by a single intradermal (ID) injection of 50 μg mBSA/20 μl saline in the right footpad, with 20 μl saline injected in the left footpad serving as control (Santos, 2001). Mice were killed 24 h later and footpad swelling quantified using micro calipers (Mitutoyo, Kawasaki-shi, Japan). DTH measurements were performed by an observer blinded to mouse genotype. Results were expressed as the difference in footpad swelling between mBSA and saline-injected footpads, and expressed as change in footpad thickness (mm). Mice were treated with compound 13 at 5 and 15 mg/kg/24 h by IP injection, twice daily for 7 days prior to antigen challenge with mBSA in the footpad. Treatment with compound 13 continued for a further 24 h and changes in footpad thickness relative to control paws were measured at that time. As shown in FIG. 4, compound 13 induced a significant inhibition of DTH reactions.

### Example 7

MIF is implicated in the recruitment of leukocytes to sites of inflammation, via studies which show that MIF-deficient mice exhibit reduced interactions between leukocytes and vascular endothelium in vivo⁽¹⁸⁾. More recently, it has been demonstrated that the administration of MIF in vivo induces the recruitment of macrophages to tissue⁽¹⁹⁾, a process which first requires the induction of adherence of circulating leukocytes to the vascular endothelial cells. As will be known to those skilled in the art, the adherence of leukocytes to the endothelium in vivo can be studied using the technique of intravital microscopy^{(18,19)}. As MIF induces leukocyte adherence to vascular endothelium as measured using intravital microscopy, a compound inhibiting the Cytokine or biological activity of MIF might be expected to inhibit the effects of MIF observable using intravital microscopy.

### Methods

Mice were anesthetised with ketamine/xylazine, and the cremaster muscle was exteriorized onto an optically-clear viewing pedestal. The cremasteric microcirculation was visualized using an intravital microscope (Axioplan 2 Imaging; Carl Zeiss, Australia) with a 20× objective lens (LD Achroplan 20×/0.40 NA, Carl Zeiss, Australia) and a 10× eyepiece. Three-five postcapillary venules (25-40 μm in diameter) were examined for each experiment. Images were visualized using a video camera and recorded on video-tape for subsequent playback analysis. Recombinant human MIF (1 mg) was injected intrascrotally in 150 μL saline, prior to intravital microscopy 4 hours later. Leukocyte-endothelial cell adhesion, was assessed as described by Gregory et al⁽¹⁹⁾. Compound 13 at a dose of 30 mg/kg or vehicle were administered by intraperitoneal injection 10 minutes prior to intrascrotal injection of MIF.

### Results

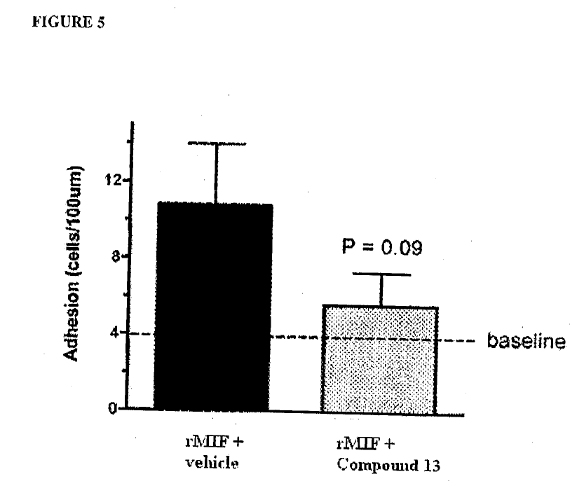

As shown in FIG. 5, MIF induced leukocyte adhesion markedly above baseline leukocyte adhesion observed without MIF injection (dotted line). MIF-induced leukocyte adhesion was reduced approximately 50% by compound 13 administration. These results are consistent with inhibition by compound 13 of in vivo effects or exogenously administered MIF.

### Determination of Lower Limits of Solubility of Compounds

An important physicochemical characteristic of pharmaceutical compounds is that die aqueous solubility of the compound is sufficiently high to allow dosing of humans with a pharmacologically active dose. Compounds with only limited aqueous solubility may be less suitable for development as a human therapeutic.

### Methods

Lower limits of aqueous compound solubility were determined in a nepholometer in phosphate-buffered saline containing 0.005% (v/v) P20 and a final concentration of 5% DMSO. Briefly, compounds were initially dissolved in DMSO as a 10 mM stock solution and diluted to 1 mM and 0.5 mM working solutions with neat DMSO. The compounds were then titrated in DMSO and a constant volume of DMSO stock added to filtered PBS/P20 solution so that the final DMSO concentration was 5%. The solubility was then determined in clear, flat-bottom 96-well plates using the nephelometer and reported as the concentration range at which the compound begins to precipitate from solution.

### Results

The results in Table 4 show that these compounds have excellent solubilities which would support dosing in humans in the uM drug range.

TABLE 4
Solubility assessment of compounds using nephelometry

| | Solubility Lower Limit |
|---|---|
| Example | Range (ug/ml) |
| 6 | 67-250 |
| 1 | 18-63 |
| 4 | >140 |
| 14 | >140 |
| 15 | 77-250 |

Throughout tills specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

All publications mentioned in this specification are herein incorporated by reference. Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed in Australia or elsewhere before the priority date of each claim of this application.

It will be appreciated by persons skilled in the art that numerous variations and/or departing from the spirit or scope of the invention as broadly described. The present embodiments are, therefore, to be considered in all respects as illustrative and not restrictive.

### REFERENCES

1. David, J, Proc. Natl. Acad. Sci., USA, (1966), 56, 72-77.
2. Leiser, W. Y., et al., Proc. Natl. Acad. Sci., USA, (1989), 86, 7522-7526.
3. Leech M, Metz C N, Smith M, Weedon H, Holdsworth S R, Bucala R, et al. Macrophage migration inhibitory factor (MIF) in rheumatoid arthritis: Evidence for pro-inflammatory function and regulation by glucocorticoids. Arthritis & Rheumatism 1999; 42:1601-1608.
4. Morand E F, Bucala R, Leech M. Macrophage migration inhibitory factor (MIF): An emerging therapeutic target in rheumatoid arthritis. Arthritis & Rheumatism 2003; 48:291-299.
5. Calandra T, Bernhagen J, Metz C N, Spiegel L A, Bacher M, Donnelly T, et al. MIF as a glucocorticoid-induced modulator of cytokine production. Nature 1995; 377:68-71.
6. Donnelly S C, Haslett C, Reid P T, Grant I S, Wallace W A H, Metz C N. I. Regulatory role for macrophage migration inhibitory factor in acute respiratory distress syndrome. Nature Medicine 1997; 3:320-323.
7. Bacher M, Metz C N, Calandra T, Mayer K, Chesney J, Lohoff M, et al. An essential regulatory role for macrophage migration inhibitory factor in T-cell activation. Proceedings of the National Academy of Sciences USA 1996; 3:7849-7854.
8. Santos L L, Hall P, Metz C N, Bucala R, Morand E F. Role of macrophage migration inhibitory factor (MIF) in murine antigen-induced arthritis: Interaction with glucocorticoids. Clin. Exp. Immunol. 2001; 123:309-314.
9. Leech M, Santos L L, Metz C, Holdsworth S R, Bucala R, Morand E F. Control of macrophage migration inhibitory factor (MIF) by endogenous glucocorticoids in rat adjuvant arthritis. Arthritis & Rheumatism 2000; 43:827-833.
10. Bucala R. MIF rediscovered: cytokine, pituitary hormone, and glucocorticoid-induced regulator of the immune response: FASEB. J. 1996; 10:1607-1613.
11. Sabroe I. Pease J E, Williams T J, Asthma and MIF: innately Th1 and Th2. Clin Exp Allergy 2000; 30(9): 1194-6.
12. Roger, T., J. David, M. P. Glauser, and T. Calandra. 2001. MIF regulates innate immune responses through modulation of Toll-like receptor 4. Nature 414:920.
13. Sampey, A. V., P. H. Hall, R. A. Mitchell, C. N. Mete, and E. F. Morand. 2001. Regulation of synoviocyte phospholipase A2 and cyclooxygenase 2 by macrophage migration inhibitory factor. Arthritis Rheum 44:1273.
14. Bernhagen, J., T. Calandra, R. A, Mitchell, S. B. Martin, K. J. Tracey, W. Voelter, K. R. Manogue, A. Cerami, and R. Bucala. 1993. MIF is a pituitary-derived cytokine that potentiates lethal endotoxaemia. Nature 365:756.
15. Rosengren, F., R. Bucala, P. Aman, L. Jacobsson, G. Odh, C. N. Metz, and H. Rorsman. 1996. The immunoregulatory mediator macrophage migration inhibitory factor (MIF) catalyzes a tautomerization reaction. Mol Med 2:143-149.
16. Santos, L. L., P. Hall, C. N. Metz, R. Bucala, and E. F. Morand. 2001. Role of macrophage migration inhibitory factor (MIF) in murine antigen-induced arthritis: Interaction with glucocorticoids, Clin Exp Immunol 123:309-314.
17. Bernhagen, J., M. Bacher, T. Calandra, C. N. Metz, S. B. Doty, T. Donnelly, and R. Bucala, 1996. An essential role for macrophage migration inhibitory factor in the tuberculin delayed-type hypersensitivity reaction. J Exp Med 183:277-282.
18. Gregory, S. L., M. T. Leech, J. R. David, Y. H. Yang, A. Dacumos, and M. J. Hickey. 2004. Reduced leukocyte-endothelial cell interactions in the inflamed microcirculation of macrophage migration inhibitory factor-deficient mice. Arthritis Rheum 50:3023-3034.
19. Gregory, J. L., E. F. Morand, S. J. McKeown, J. A. Ralph, P. Hall, Y. H. Yang, S. R. McColl, and M. J. Hickey. 2006. Macrophage Migration Inhibitory Factor Induces Macrophage Recruitment via CC Chemokine Ligand 2. J Immunol 177:8072-8079.
20. Aeberli D, Yang Y H, Mansell A, Santos L, Leech M, Morand E F; Macrophage migration inhibitory factor modulates glucocorticoid sensitivity in macrophages via effects on MAP kinase phosphatase-1 and p38 MAP kinase. FEBS Lett 2006, 580:974-981.
21. Roger T, Chanson A L, Knaup-Reymond M, Calandra T: Macrophage migration inhibitory factor promotes innate immune responses by suppressing glucocorticoid-induced expression of mitogen-activated protein kinase phosphatase-1. Eur J Immunol 2005, 35:3405-3413.
22. Gregory J L, Morand E F, McKeown S J, Ralph J A, Hall P, Yang Y H, McColl S R, Hickey M J: Macrophage Migration Inhibitory Factor Induces Macrophage Recruitment via CC Chemokine Ligand 2. J Immunol 2006, 177:8072-8079.
23. Gregory J L, Leech M T, David J R, Yang Y H, Dacumos A, Hickey M J: Reduced leukocyte-endothelial cell interactions in the inflamed microcirculation of macrophage migration inhibitory factor-deficient mice. Arthritis Rheum 2004, 50:3023-3034.
24. Morand E F, Leech M, Bernhagen J: MIF: a new cytokine link between rheumatoid arthritis and atherosclerosis. Nat Rev Drug Discov 2006, 5:399-410.

1. A method of treating, diagnosing or preventing autoimmune diseases, tumours, or chronic or acute inflammatory diseases comprising administering a treatment, prevention or diagnostic effective amount of a compound of formula (I) or a pharmaceutically acceptable salt or prodrug thereof to a subject in need thereof wherein: 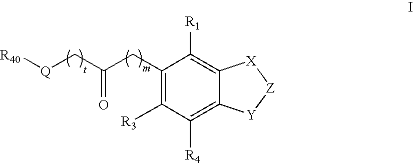
X is selected from —O—, —S—, —C(R₅)(R_{5′})— and —N(R₆)—;
Y is selected from —N(R₇)—, —O—, —S—, and —C(R₇)₂—;
Z is selected from >C═O, >C═S, >C═NR₆, >S═O and >S(O)₂;
R₁ is selected from hydrogen, C₁-C₃alkyl, (CR₅R_{5′})ₙOR₇, C(R₅R_{5′})ₙSR₇, (CR₅R_{5′})ₙN(R₆)₂ and (CR₅R_{5′})ₙhalo;
R₃ is selected from hydrogen, C₁-C₆alkyl, (CR₁₆R_{16′})ₚNR₁₄R₁₅, (CR₁₆R_{16′})ₚOR₁₇, (CR₁₆R_{16′})ₚSR₁₇, (CR₁₆R_{16′})ₚhalo, (CR₁₆R_{16′})ₚNO₂, (CR₁₆R_{16′})ₙC(O)R₂₈, (CR₁₆R_{16′})ₙC(═NR₂₄)R₂₂, (CR₁₆R_{16′})ₙS(O)R₁₇, (CR₁₆R_{16′})ₙS(O)₂R₁₇, (CR₁₆R_{16′})ₙS(O)₃R₁₇, and (CR₁₆R_{16′})ₚC(R₁₈)₃;
R₄ is selected from hydrogen, halogen, C₁-C₃alkyl, C₂-C₃alkenyl, C₂-C₃alkynyl and (CR₁₂R_{12′})ₙ(CR₁₈)₃;
each R₅ and R_{5′} is independently selected from hydrogen, C₁-C₃alkyl, halo, OR₇, SR₇ and N(R₆)₂;
each R₆ is independently selected from hydrogen, C₁-C₃alkyl and OR₇;
each R₇ is independently selected from hydrogen and C₁-C₃alkyl;
each R₁₂ and R_{12′} is independently selected from hydrogen, C₁-C₆alkyl, C₂-C₆alkenyl, C₂-C₆alkynyl, OR₂₄, SR₂₄, halo, N(R₂₄)₂, CO₂R₂₄, CN, NO₂, aryl and heterocyclyl;
each R₁₄ and R₁₅ is independently selected from hydrogen, C₁-C₃alkyl, OR₁₇, SR₁₇, and N(R₁₇)₂;
each R₁₆ and R_{16′} is independently selected from hydrogen, C₁-C₃alkyl, halo, OR₁₇, SR₁₇ and N(R₁₇)₂;
each R₁₇ is independently selected from hydrogen and C₁-C₃alkyl;
each R₁₈ is independently selected from hydrogen and halo;
R₂₂ is selected from C₁-C₆alkyl, NH₂, NH(C₁-C₆alkyl), N(C₁-C₆alkyl)₂, OR₂₉ or SR₂₉;
each R₂₄ is selected from H and C₁-C₆alkyl;
R₂₈ is selected from hydrogen, C₁-C₆alkyl, OR₂₉, SR₂₉ or N(R₂₉)₂;
each R₂₉ is independently selected from hydrogen and C₁-C₃alkyl;
Q is selected from O, S, NR₄₀, S(O)ᵤ where u is an integer from 1 to 2;
R₄₀ is selected from H, OH, and C(R₄₁R_{41′})ᵥR₄₂;
each R₄₁ and R_{41′} is independently selected from H, OH, halo, NH₂, cyano, and NO₂;
R₄₂ is independently selected from H, OR₄₃, COOR₄₃, CON(R₄₃R_{43′}), O(CO)R₄₃, aryl, and heterocyclyl;
each R₄₃ and R_{43′} is independently selected from H, C₁₋₆alkyl, benzyl, and aryl;
n=0 or an integer to 3;
m is 0 or an integer from 1 to 20;
p is 0 or an integer from 1 to 6;
t is an integer from 1 to 10; and
v is 0 or an integer from 1 to 10.

2. The method according to claim 1, wherein the autoimmune disease, tumour, or chronic or acute inflammatory disease is selected from the group consisting of: rheumatic diseases; spondyloarthropathies; crystal arthropathies; Lyme disease; polymyalgia rheumatica; connective tissue diseases; vasculitides; inflammatory conditions; sarcoidosis; vascular diseases; vascular occlusive disease; vascular stent restenosis; ocular diseases; autoimmune diseases; pulmonary diseases; cancers; renal diseases; disorders of the hypothalamic-pituitary-adrenal axis; nervous system disorders; diseases characterised by modified angiogenesis; endometrial function; complications of infective disorders; transplant rejection, graft-versus-host disease; allergic diseases; bone diseases; skin diseases; diabetes mellitus and its complications; pain, testicular dysfunctions and wound healing; gastrointestinal diseases; peptic ulceration; gastritis; oesophagitis; and liver disease.

3. The method according to claim 1, wherein MIF cytokine or biological activity is implicated in the disease or condition.

4. The method according to claim 1, wherein the disease or condition is selected from the group consisting of rheumatoid arthritis, systemic lupus erythematosus, ulcerative colitis, Crohn's disease, multiple sclerosis, psoriasis, uveitis, diabetes mellitus, glomerulonephritis, atherosclerotic vascular disease and infarction, asthma and chronic obstructive pulmonary disease.

5. The method according to claim 1, wherein Q is S.

6. The method according to claim 1, wherein R₄₀ is C(R₄₁R_{41′})vR₄₂ and R₄₂ is COOR₄₃.

7. The method according to claim 6, wherein R₄₃ is hydrogen or C₁-C₆alkyl.

8. The method according to claim 6, wherein R₄₃ is methyl.

9. The method according to claim 1, wherein the compound of formula (I) is selected from the group consisting of:

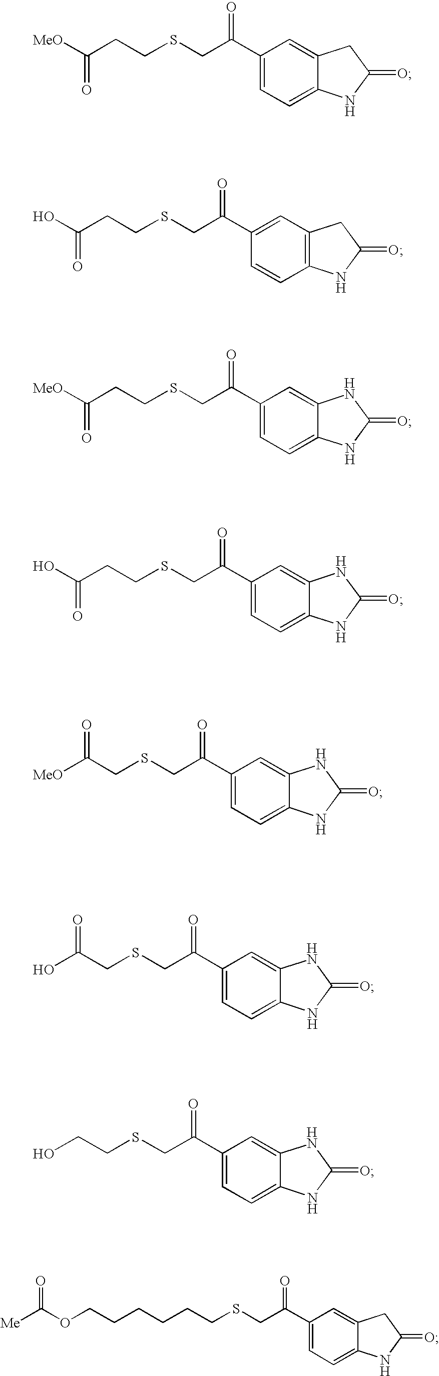

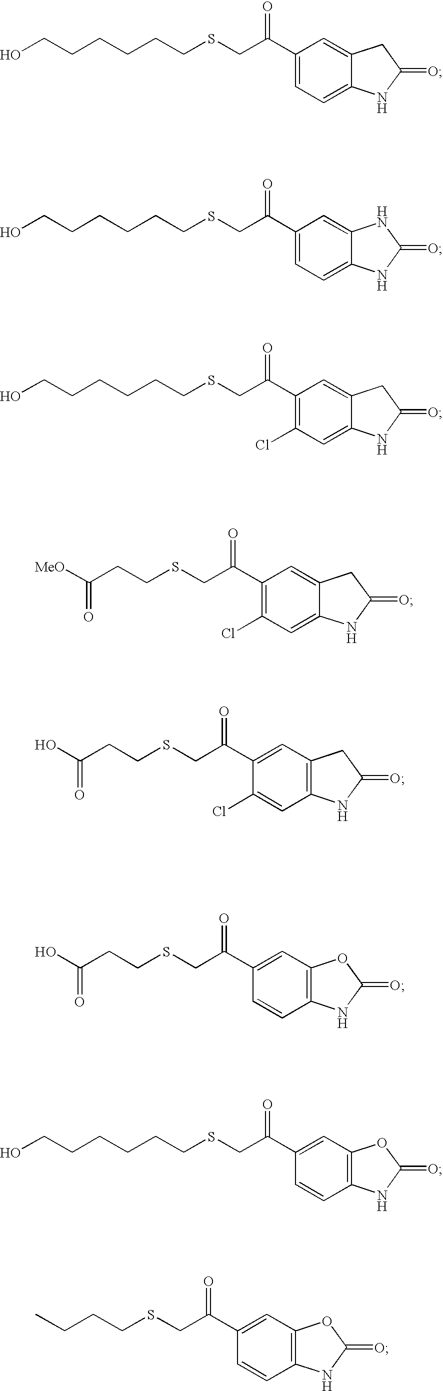

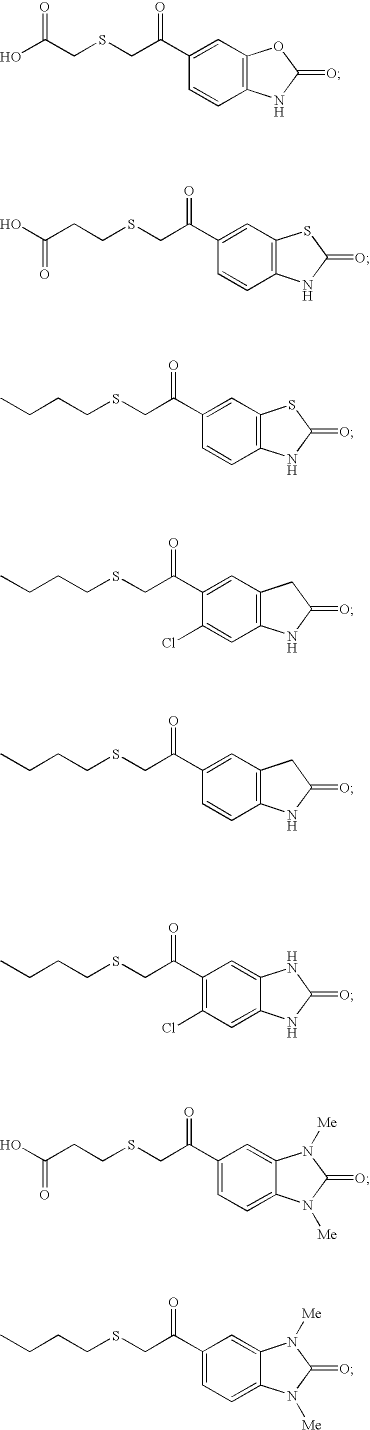

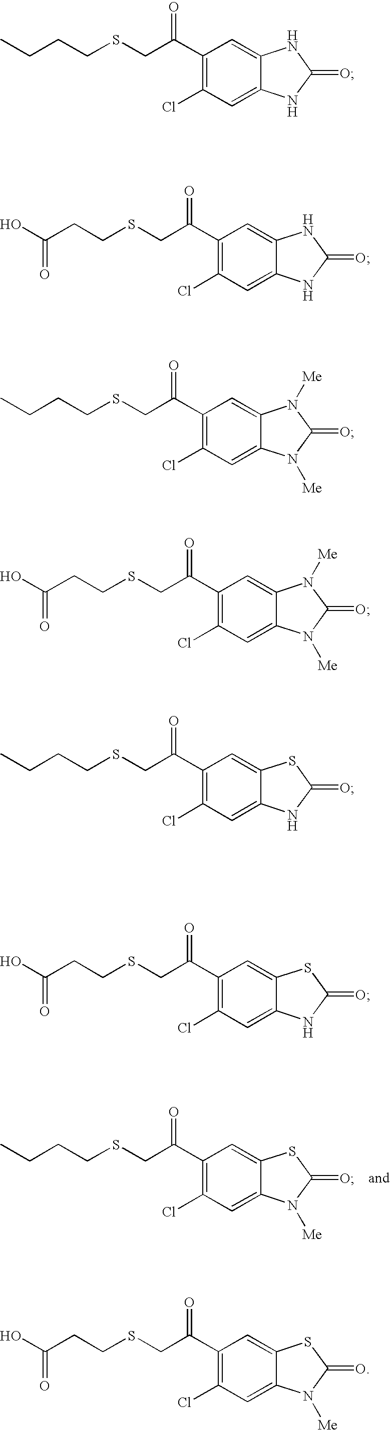

10. The method according to claim 9, wherein the compound of formula (I) is selected from the group consisting of:

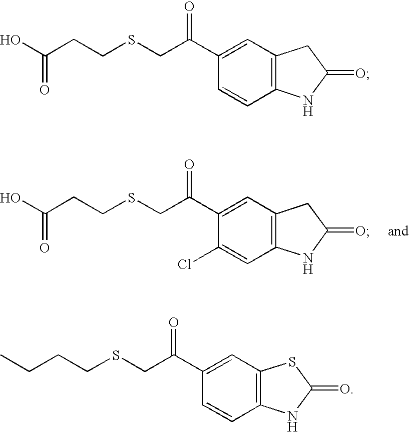

11. A compound selected from the group consisting of:

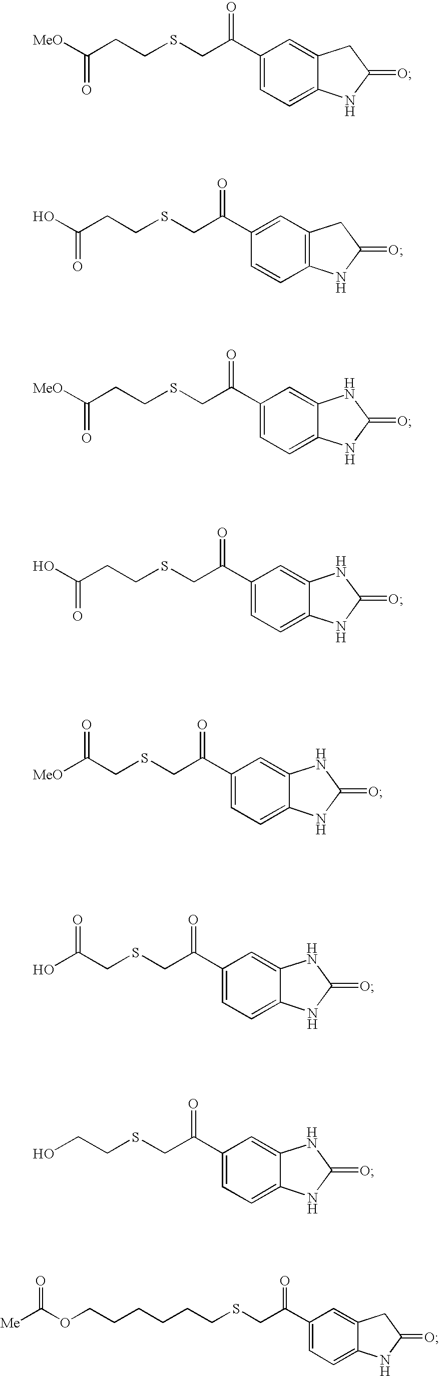

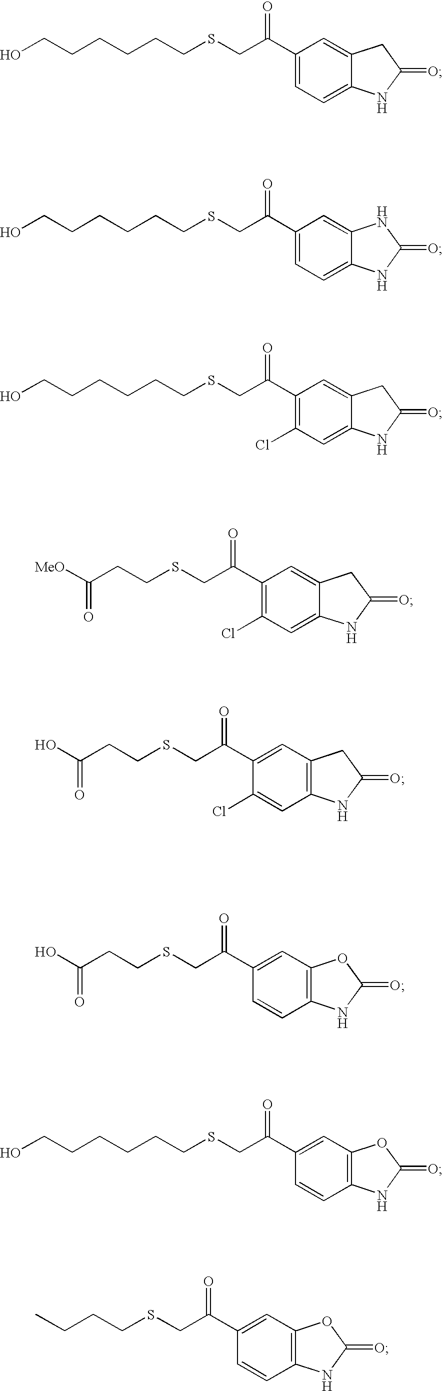

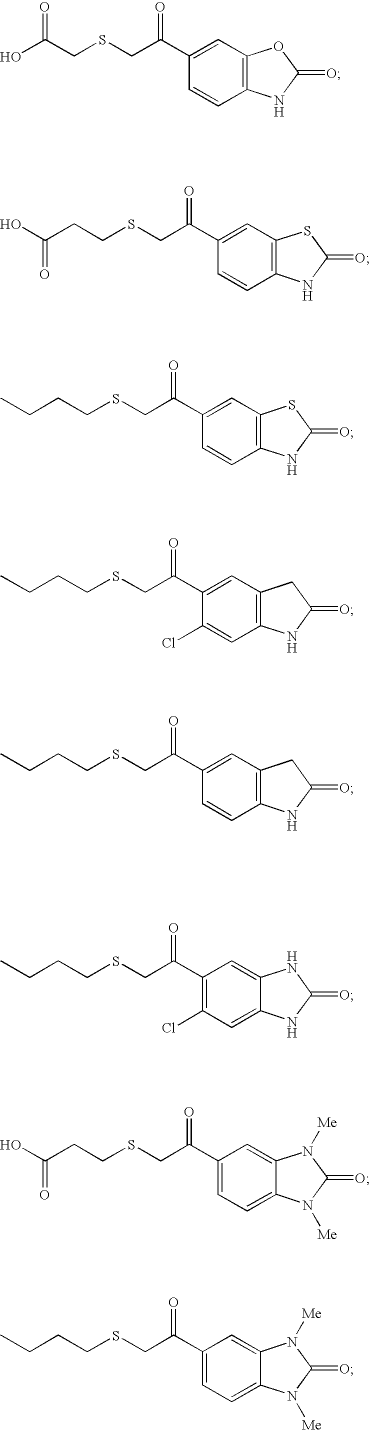

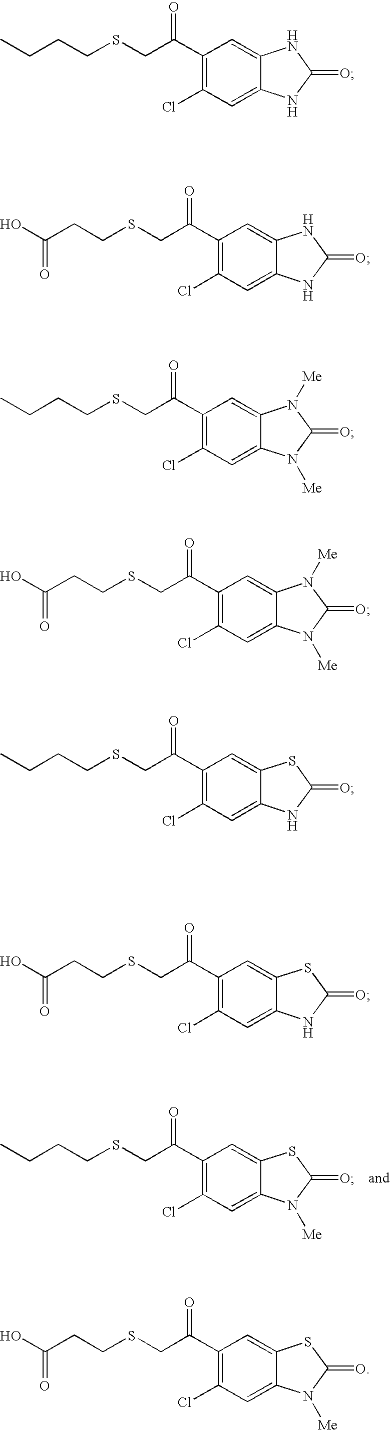

12. A compound of Formula (II) or a pharmaceutically acceptable salt or prodrug thereof wherein: 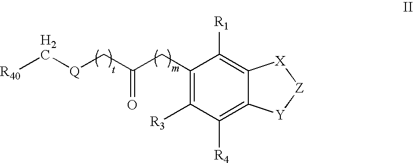
X is selected from —O—, —S—, —C(R₅)(R_{5′})— and —N(R₆)—;
Y is selected from —N(R₇)—, —O—, and —S—;
Z is selected from >C═O, >C═S, and >C═NR₆;
R₁ is selected from hydrogen, C₁-C₃alkyl, (CR₅R_{5′})ₙOR₇, C(R₅R_{5′})ₙSR₇, (CR₅R_{5′})ₙN(R₆)₂ and (CR₅R_{5′})ₙhalo;
R₃ is selected from hydrogen, C₁-C₆alkyl, (CR₁₆R_{16′})ₚNR₁₄R₁₅, (CR₁₆R_{16′})ₚOR₁₇, (CR₁₆R_{16′})ₚSR₁₇, (CR₁₆R_{16′})ₚhalo, (CR₁₆R_{16′})ₚNO₂, (CR₁₆R_{16′})ₙC(O)R₂₈, (CR₁₆R_{16′})ₙC(═NR₂₄)R₂₂, (CR₁₆R_{16′})ₙS(O)R₁₇, (CR₁₆R_{16′})ₙS(O)₂R₁₇, (CR₁₆R_{16′})ₙS(O)₃R₁₇, and (CR₁₆R_{16′})ₚC(R₁₈)₃;
R₄ is selected from hydrogen, halogen, C₁-C₃alkyl, C₂-C₃alkenyl, C₂-C₃alkynyl and (CR₁₂R_{12′})ₙ(CR₁₈)₃;
each R₅ and R_{5′} is independently selected from hydrogen, C₁-C₃alkyl, halo, OR₇, SR₇ and N(R₆)₂;
each R₆ is independently selected from hydrogen, C₁-C₃alkyl and OR₇;
each R₇ is independently selected from hydrogen and C₁-C₃alkyl;
each R₁₂ and R_{12′} is independently selected from hydrogen, C₁-C₆alkyl, C₂-C₆alkenyl, C₂-C₆alkynyl, OR₂₄, SR₂₄, halo, N(R₂₄)₂, CO₂R₂₄, CN, NO₂, aryl and heterocyclyl;
each R₁₄ and R₁₅ is independently selected from hydrogen, C₁-C₃alkyl, OR₁₇, SR₁₇, and N(R₁₇)₂;
each R₁₆ and R_{16′} is independently selected from hydrogen, C₁-C₃alkyl, halo, OR₁₇, SR₁₇ and N(R₁₇)₂;
each R₁₇ is independently selected from hydrogen and C₁-C₃alkyl;
each R₁₈ is independently selected from hydrogen and halo;
R₂₂ is selected from C₁-C₆alkyl, NH₂, NH(C₁-C₆alkyl), N(C₁-C₆alkyl)₂, OR₂₉ or SR₂₉;
each R₂₄ is selected from H and C₁-C₆alkyl;
R₂₈ is selected from hydrogen, C₁-C₆alkyl, OR₂₉, SR₂₉ or N(R₂₉)₂;
each R₂₉ is independently selected from hydrogen and C₁-C₃alkyl;
Q is selected from O, S, S(O)ᵤ where u is an integer from 1 to 2;
R₄₀ is selected from H, OH, and C(R₄₁R_{41′})ᵥR₄₂;
each R₄₁ and R_{41′} is independently selected from H, OH, halo, NH₂, CN and NO₂;
R₄₂ is selected from H, OR₄₃, COOR₄₃, CON(R₄₃R_{43′}), O(CO)R₄₃, N(R₄₃R_{43′}), aryl, and heterocyclyl;
each R₄₃ and R_{43′} is independently selected from H, C₁₋₆ alkyl, and benzyl;
n is 0 or 1 to 3;
m is 0 or an integer from 1 to 8;
p is 0 or an integer from 1 to 6;
t is an integer from 1 to 10; and
v is 0 or an integer from 1 to 10;
provided that the compound is not

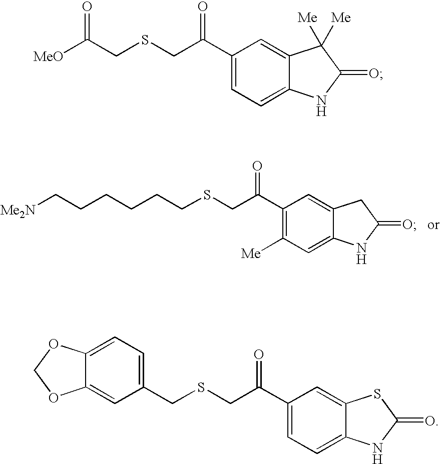

13. The compound according to claim 12, wherein Q is S.

14. The compound according to claim 12, wherein R₄₀ is C(R₄₁R_{41′})vR₄₂ and R₄₂ is COOR₄₃.

15. The compound according to claim 14, wherein R₄₃ is hydrogen or C₁-C₆alkyl.

16. The compound according to claim 14, wherein R₄₃ is methyl.

17. A compound of Formula III or a pharmaceutically acceptable salt or prodrug thereof wherein: 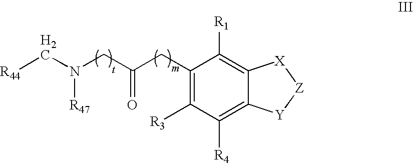
X is selected from —O—, —S—, —C(R₅)(R_{5′})— and —N(R₆)—;
Y is selected from —N(R₇), —O—, and —S—;
Z is selected from >C═O, >C═S, and >C═NR₆;
R₁ is selected from hydrogen, C₁-C₃alkyl, (CR₅R_{5′})ₙOR₇, C(R₅R_{5′})ₙSR₇, (CR₅R_{5′})ₙN(R₆)₂ and (CR₅R_{5′})ₙhalo;
R₃ is selected from hydrogen, C₁-C₆alkyl, (CR₁₆R_{16′})ₚNR₁₄R₁₅, (CR₁₆R_{16′})ₚOR₁₇, (CR₁₆R_{16′})ₚSR₁₇, (CR₁₆R_{16′})ₚhalo, (CR₁₆R_{16′})ₚNO₂, (CR₁₆R_{16′})ₙC(O)R₂₈, (CR₁₆R_{16′})ₙC(═NR₂₄)R₂₂, (CR₁₆R_{16′})S(O)R₁₇, (CR₁₆R_{16′})ₙS(O)₂R₁₇, (CR₁₆R_{16′})ₙS(O)₃R₁₇, and (CR₁₆R_{16′})ₚC(R₁₈)₃;
R₄ is selected from hydrogen, halogen, C₁-C₃alkyl, C₂-C₃alkenyl, C₂-C₃alkynyl and (CR₁₂R_{12′})ₙ(CR₁₈)₃;
each R₅ and R_{5′} is independently selected from hydrogen, C₁-C₃alkyl, halo, OR₇, SR₇ and N(R₆)₂;
each R₆ is independently selected from hydrogen, C₁-C₃alkyl and OR₇;
each R₇ is independently selected from hydrogen and C₁-C₃alkyl;
each R₁₂ and R_{12′} is independently selected from hydrogen, C₁-C₆alkyl, C₂-C₆alkenyl, C₂-C₆alkynyl, OR₂₄, SR₂₄, halo, N(R₂₄)₂, CO₂R₂₄, CN, NO₂, aryl and heterocyclyl;
each R₁₄ and R₁₅ are independently selected from hydrogen, C₁-C₃alkyl, OR₁₇, SR₁₇, and N(R₁₇)₂;
each R₁₆ and R_{16′} is independently selected from hydrogen, C₁-C₃alkyl, halo, OR₁₇, SR₁₇ and N(R₁₇)₂;
each R₁₇ is independently selected from hydrogen and C₁-C₃alkyl;
each R₁₈ is independently selected from hydrogen and halo;
R₂₂ is selected from C₁-C₆alkyl, NH₂, NH(C₁-C₆alkyl), N(C₁-C₆alkyl)₂, OR₂₉ or SR₂₉;
each R₂₄ is selected from H and C₁-C₆alkyl;
R₂₈ is selected from hydrogen, C₁-C₆alkyl, OR₂₉, SR₂₉ or N(R₂₉)₂;
each R₂₉ is independently selected from hydrogen and C₁-C₃alkyl;
R₄₄ is selected from OH, C(R₄₅R_{45′})ᵥR₄₆;
each R₄₅ and R_{45′} is independently selected from H, OH, halo, NH₂, CN, NO₂;
each R₄₆ is selected from COOR₄₇, CON(R₄₇R_{47′}), O(CO)R₄₇, N(R₄₇R_{47′});
each R₄₇ and R_{47′} is independently selected from H, C₁₋₆ alkyl, benzyl;
wherein when v is greater than 1, R₄₆ can be OR₄₇;
wherein when v is greater than 2, R₄₆ can be H;
n is 0 or 1 to 3;
m is 0 or an integer from 1 to 8;
p is 0 or an integer from 1 to 6;
t is an integer from 1 to 10; and
v is 0 or an integer from 1 to 10;
provided that the compound is not

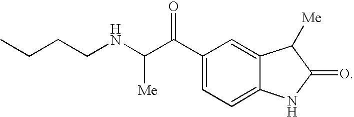

18. A use of a compound of Formula (I) as defined in claim 1, or a pharmaceutically acceptable salt or prodrug thereof in the manufacture of a medicament for treating, diagnosing or preventing autoimmune disease, tumour, or chronic or acute inflammatory disease selected from the group consisting of: rheumatic diseases; spondyloarthropathies; crystal arthropathies; Lyme disease; polymyalgia rheumatica; connective tissue diseases; vasculitides; inflammatory conditions; sarcoidosis; vascular diseases; vascular occlusive disease; vascular stent restenosis; ocular diseases; autoimmune diseases; pulmonary diseases; cancers; renal diseases; disorders of the hypothalamic-pituitary-adrenal axis; nervous system disorders; diseases characterised by modified angiogenesis; endometrial function; complications of infective disorders; transplant rejection, graft-versus-host disease; allergic diseases; bone diseases; skin diseases; diabetes mellitus and its complications; pain, testicular dysfunctions and wound healing; gastrointestinal diseases; peptic ulceration; gastritis; oesophagitis; and liver disease.

19. A use according to claim 18, wherein MIF cytokine or biological activity is implicated in the disease or condition.

20. A use according to claim 18, wherein the disease or condition is selected from the group consisting of rheumatoid arthritis, systemic lupus erythematosus, ulcerative colitis, Crohn's disease, multiple sclerosis, psoriasis, uveitis, diabetes mellitus, glomerulonephritis, atherosclerotic vascular disease and infarction, asthma and chronic obstructive pulmonary disease.

21. A pharmaceutical composition comprising a compound according to any one of claims 11, 12 or 17 and a pharmaceutically acceptable carrier, diluent or excipient.

22. A method of inhibiting cytokine or biological activity of MIF comprising contacting MIF with a cytokine or biological inhibiting amount of a compound of Formula (I) as defined in claim 1, or a pharmaceutically acceptable salt or prodrug thereof.

23. A method of treating, preventing or diagnosing a disease or condition wherein MIF cytokine or biological activity is implicated comprising the administration of a treatment, prevention or diagnostic effective amount of a compound of Formula (I) as defined in claim 1, or a pharmaceutically acceptable salt or prodrug thereof to a subject in need thereof.

24. A method of treating or preventing a disease or condition wherein MIF cytokine or biological activity is implicated comprising:
administering to a mammal a compound of Formula (I) as defined in claim 1, or a pharmaceutically acceptable salt or prodrug thereof and a second therapeutic agent.

25. A method of prophylaxis or treatment of a disease or condition for which treatment with a glucocorticoid is indicated, said method comprising:
administering to a mammal a glucocorticoid and a compound of Formula (I) as defined in claim 1, or a pharmaceutically acceptable salt or prodrug thereof.

26. A method of treating steroid-resistant diseases comprising:
administering to a mammal a glucocorticoid and a compound of Formula (I) as defined in claim 1, or a pharmaceutically acceptable salt or prodrug thereof.

27. A method of enhancing the effect of a glucocorticoid in mammals comprising administering a compound of Formula (I) as defined in claim 1, or a pharmaceutically acceptable salt or prodrug thereof simultaneously, separately or sequentially with said glucocorticoid.

28. A pharmaceutical composition comprising a glucocorticoid and a compound of Formula (I) as defined in claim 1, or a pharmaceutically acceptable salt or prodrug thereof.

29. A use of a glucocorticoid in the manufacture of a medicament for administration with a compound of Formula (I) as defined in claim 1, or a pharmaceutically acceptable salt or prodrug thereof for the treatment or prophylaxis of a disease or condition for which treatment with a glucocorticoid is indicated.

30. A use of a compound of Formula (I) as defined in claim 1, or a pharmaceutically acceptable salt or prodrug thereof in the manufacture of a medicament for administration with a glucocorticoid for the treatment or prophylaxis of a disease or condition for which treatment of a glucocorticoid is indicated.

31. A use of a glucocorticoid and a compound of Formula (I) as defined in claim 1, or a pharmaceutically acceptable salt or prodrug thereof in the manufacture of a medicament for the treatment or prophylaxis of a disease or condition for which treatment with a glucocorticoid is indicated.

32. An implantable device comprising:
(i) a reservoir containing at least one compound of Formula (I) as defined in claim 1, or a pharmaceutically acceptable salt or prodrug thereof; and
(ii) means to release or elute the at least one compound of Formula (I) from the reservoir.

33. The implantable device according to claim 32, wherein the implantable device is a stent.

34. The implantable device for inhibiting the cytokine or biological activity of MIF in a subject comprising the step of implanting an implantable device according to claim 32, in a subject.
